SEQUENCE LISTING (69 sequences)

SEQ ID NO: 1 - DNA, 1997 bp, Homo sapiens
  CDS (352)..(1608)
cccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattat

SEQ ID NO: 2 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 3 - DNA, 2029 bp, Homo sapiens
  CDS (411)..(1475)
gttgggttccagctttctgtagctgtaagcattggtggccacaccacctccttacaaagcaactagaacctgcggcatacattggagagatttttttaattttctggacatgaagtaaatttagagtgctttctaatttcaggtagaagacatgtccaccttctgattatttttggagaacattttgatttttttcatctctctctccccacccctaagattgtgcaaaaaaagcgtaccttgcctaattgaaataatttcattggattttgatcagaactgattatttggttttctgtgtgaagttttgaggtttcaaactttccttctggagaatgccttttgaaacaattttctctagctgcctgatgtcaactgcttagtaatcagtggatattgaaatattcaaaatgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttgattcagcgttccaagttccccatccctgtcatttttaacagcatgctgctttgccaagttgctgtcactgtttttttcccaggtgttaaaaaaaaaatccattttacacagcaccacagtgaatccagaccaaccttccattcacaccagctaaggagtccctggttcattgatggatgtcttctagctgcagatgcctctgcgcaccaaggaatggagaggaggggacccatgtaatccttttgtttagttttgtttttgttttttggtgaatgagaaaggtgtgctggtcatggaatggcaggtgtcatatgactgattactcagagcagatgaggaaaactgtagtctctgagtcctttgctaatcgcaactcttgtgaattattctgattcttttttatgcagaatttgattcgtatgatcagtactgactttctgattactgtccagcttatagtcttccagtttaatgaactaccatctgatgtttcatatttaagtgtatttaaagaaaataaacaccattattcaagccaaaaaaaaaaaaaaaaaa

SEQ ID NO: 4 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 5 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 6 - DNA, 576 bp, Homo sapiens
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 7 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 8 - protein, 188 aa, Homo sapiens [sequence omitted]

SEQ ID NO: 9 - DNA, 106 bp, Homo sapiens
gtttttttattttccagaaaatcagttcgaggaaagggaaaggggcaaaaacgaaagcgcaagaaatcccggtataagtcctggagcgtgtacgttggtgcccgct

SEQ ID NO: 10 - protein, 215 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 11 - DNA, 166 bp, Homo sapiens
cttttgcctttttgcagtccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcaggttggttcccagaggca

SEQ ID NO: 12 - protein, 161 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRQLELNERTCRCDKPRR

SEQ ID NO: 13 - DNA, 76 bp, Homo sapiens
ttttccatttccctcagatgtgacaagccgaggcggtgagccgggcaggaggaaggagcctccctcagggtttcgg

SEQ ID NO: 14 - protein, 181 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVQLELNERTCRCDKPRR

SEQ ID NO: 15 - protein, 221 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRHSPGRQSPDMPGDFRADAPSFLPPRRSLPMLFRMEWGCALTGSQSAVWPSSPVPEEIPRMHPGRNGKKQQRKPLREKMKPERCGDAVPRR

SEQ ID NO: 16 - DNA, 22 bp, Artificial sequence
  Synthetic primer
acattggtgtgcacctccaagc

SEQ ID NO: 17 - DNA, 27 bp, Artificial sequence
  Synthetic primer
aataatggaatgaacttgtctgtaaac

SEQ ID NO: 18 - DNA, 24 bp, Artificial sequence
  Synthetic primer
aaatcagttcgaggaaagggaaag

SEQ ID NO: 19 - DNA, 21 bp, Artificial sequence
  Synthetic primer
ccctgtgggccttgctcagag

SEQ ID NO: 20 - DNA, 35 bp, Artificial sequence
  Synthetic primer
ccatgctcgagagtctttcctggtgagagatctgg

SEQ ID NO: 21 - protein, 682 aa, Homo sapiens
MVSWGRFICLVVVTMATLSLARPSFSLVEDTTLEPEDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRIPPNPSLMSIFRK

SEQ ID NO: 22 - protein, 208 aa, Homo sapiens
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH

SEQ ID NO: 23 - protein, 234 aa, Homo sapiens [sequence omitted]

SEQ ID NO: 24 - protein, 452 aa, Homo sapiens
MDSGRRLGPEKWIRRLRRMESESESGAAADTPPLETLSFHGDEEIIEVVELDPGPPDPDDLAQEMEDVDFEEEEEEEGNEEGWVLEPQEGVVGSMEGPDDSEVTFALHSASVFCVSLDPKTNTLAVTGGEDDKAFVWRLSDGELLFECAGHKDSVTCAGFSHDSTLVATGDMSGLLKVWQVDTKEEVWSFEAGDLEWMEWHPRAPVLLAGTADGNTWMWKVPNGDCKTFQGPNCPATCGRVLPDGKRAVVGYEDGTIRIWDLKQGSPIHVLKGTEGHQGPLTCVAANQDGSLILTGSVDCQAKLVSATTGKVVGVFRPETVASQPSLGEGEESESNSVESLGFCSVMPLAAVGYLDGTLAIYDLATQTLRHQCQHQSGIVQLLWEAGTAVVYTCSLDGIVRLWDARTGRLLTDYRGHTAEILDFALSKDASLVVTTSGDHKAKVFCVQRPDR

SEQ ID NO: 25 - protein, 4563 aa, Homo sapiens
  misc_feature (32)..(126): heparin binding domain
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYKNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEVLKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPQTDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSCDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVLENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDVLEHVKHFVINLIGDFEVAEKINAFRAKVHELIERYEVDQQIQVLMDKLVELAHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMLNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPHFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVAKTTKAEIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFITPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTLAHRDFSAEYEEDGKFEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQKFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFNDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 26 - protein, 317 aa, Homo sapiens [sequence omitted]

SEQ ID NO: 27 - protein, 268 aa, Homo sapiens
MSLSFLLLLFFSHLILSAWAHGEKRLAPKGQPGPAATDRNPRGSSSRQSSSSAMSSSSASSSPAASLGSQGSGLEQSSFQWSPSGRRTGSLYCRVGIGFHLQIYPDGKVNGSHEANMLSVLEIFAVSQGIVGIRGVFSNKFLAMSKKGKLHASAKFTDDCKFRERFQENSYNTYASAIHRTEKTGREWYVALNKRGKAKRGCSPRVKPQHISTHFLPRFKQSEQPELSFTVTVPEKKNPPSPIKSKIPLSAPRKNTNSVKYRLKFRFG

SEQ ID NO: 28 - protein, 55 aa, Homo sapiens
ARQENPCGPCSERRKHLFVQDPQTCKCSCKNTDSRCKARQLELNERTCRCDKPRR

SEQ ID NO: 29 - protein, 271 aa, Homo sapiens
AIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKT

SEQ ID NO: 30 - protein, 225 aa, Homo sapiens
IVGGRKARPRQFPFLASIQNQGRHFCGGALIHARFVMTAASCFQSQNPGVSTVVLGAYDLRRRERQSRQTFSISSMSENGYDPQQNLNDLMLLQLDREANLTSSVTILPLPLQNATVEAGTRCQVAGWGSQRSGGRLSRFPRFVNVTVTPEDQCRPNNVCTGVLTRRGGICNGDGGTPLVCEGLAHGVASFSLGPCGRGPDFFTRVALFRDWIDGVLNNPGPGPA

SEQ ID NO: 31 - protein, 374 aa, Homo sapiens
AVTTIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTGQEALSQTTISWAPFQDTSEYIISCHPVGTDEEPLQFVPGTSTSATLTGLTRGATYNIIVEALKDQQRHKVREEVVTVGNSVNEGLNQPTDDSCFDPYTV

SEQ ID NO: 32 - protein, 51 aa, Homo sapiens
CHPGYHGERCHGLSLPVENHLYTYDHTTILAVVAVVLSSVCLLVIVGLLMF

SEQ ID NO: 33 - protein, 23 aa, Homo sapiens
YHRRGGYDVENEEKVKLGMTNSH

SEQ ID NO: 34 - protein, 58 aa, Homo sapiens
FILQATSTGHTKQGGAREKKEERQGAREEEGPMSSEIQGLLHPWRMQICEGAPGSLLH

SEQ ID NO: 35 - protein, 317 aa, Homo sapiens [sequence omitted]

SEQ ID NO: 36 - protein, 316 aa, Homo sapiens
MRQPLSWGRWRAMLARTYGPGPSAGYRWASGAQGYVRNPPVGACDLQGELDRFGGISVRLARLDALDRLDAAAFQKGLQAAVQQWRSEGRTAVWLHIPILQSRFIAPAASLGFCFHHAESDSSTLTLWLREGPSRLPGYASHQVGVAGAVFDESTRKILVVQDRNKLKNMWKFPGGLSEPEEDIGDTAVREVFEETGIKSEFRSVLSIRQQHTNPGAFGKSDMYIICRLKPYSFTINFCQEECLRCEWMDLNDLAKTENTTPITSRVARLLLYGYREGFDKIDLTVEELPAVYTGLFYKLYHKELPENYKTMKGID

SEQ ID NO: 37 - protein, 147 aa, Homo sapiens
MWKFPGGLSEPEEDIGDTAVREVFEETGIKSEFRSVLSIRQQHTNPGAFGKSDMYIICRLKPYSFTINFCQEECLRCEWMDLNDLAKTENTTPITSRVARLLLYGYREGFDKIDLTVEELPAVYTGLFYKLYHKELPENYKTMKGID

SEQ ID NO: 38 - protein, 251 aa, Homo sapiens
MTRLTVLALLAGLLASSRAGSSPLLDIVGGRKARPRQFPFLASIQNQGRHFCGGALIHARFVMTAASCFQSQNPGVSTVVLGAYDLRRRERQSRQTFSISSMSENGYDPQQNLNDLMLLQLDREANLTSSVTILPLPLQNATVEAGTRCQVAGWGSQRSGGRLSRFPRFVNVTVTPEDQCRPNNVCTGVLTRRGGICNGDGGTPLVCEGLAHGVASFSLGPCGRGPDFFTRVALFRDWIDGVLNNPGPGPA

SEQ ID NO: 39 - protein, 345 aa, Homo sapiens
MISPVLILFSSFLCHVAIAGRTCPKPDDLPFSTVVPLKTFYEPGEEITYSCKPGYVSRGGMRKFICPLTGLWPINTLKCTPRVCPFAGILENGAVRYTTFEYPNTISFSCNTGFYLNGADSAKCTEEGKWSPELPVCAPIICPPPSIPTFATLRVYKPSAGNNSLYRDTAVFECLPQHAMFGNDTITCTTHGNWTKLPECREVKCPFPSRPDNGFVNYPAKPTLYYKDKATFGCHDGYSLDGPEEIECTKLGNWSAMPSCKASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 40 - protein, 464 aa, Homo sapiens
MYSNVIGTVTSGKRKVYLLSLLLIGFWDCVTCHGSPVDICTAKPRDIPMNPMCIYRSPEKKATEDEGSEQKIPEATNRRVWELSKANSRFATTFYQHLADSKNDNDNIFLSPLSISTAFAMTKLGACNDTLQQLMEVFKFDTISEKTSDQIHFFFAKLNCRLYRKANKSSKLVSANRLFGDKSLTFNETYQDISELVYGAKLQPLDFKENAEQSRAAINKWVSNKTEGRITDVIPSEAINELTVLVLVNTIYFKGLWKSKFSPENTRKELFYKADGESCSASMMYQEGKFRYRRVAEGTQVLELPFKGDDITMVLILPKPEKSLAKVEKELTPEVLQEWLDELEEMMLVVHMPRFRIEDGFSLKEQLQDMGLVDLFSPEKSKLPGIVAEGRDDLYVSDAFHKAFLEVNEEGSEAAASTAVVIAGRSLNPNRVTFKANRPFLVFIREVPLNTIIFMGRVANPCVK

SEQ ID NO: 41 - protein, 240 aa, Homo sapiens
MSRSNRQKEYKCGDLVFAKMKGYPHWPARIDEMPEAAVKSTANKYQVFFFGTHETAFLGPKDLFPYEESKEKFGKPNKRKGFSEGLWEIENNPTVKASGYQSSQKKSCVEEPEPEPEAAEGDGDKKGNAEGSSDEEGKLVIDEPAKEKNEKGALKRRAGDLLEDSPKRPKEAENPEGEEKEAATLEVERPLPMEVEKNSTPSEPGSGRGPPQEEEEEEDEEEEATKEDAEAPGIRDHESL

SEQ ID NO: 42 - protein, 525 aa, Homo sapiens
MKALIAALLLITLQYSCAVSPTDCSAVEPEAEKALDLINKRRRDGYLFQLLRIADAHLDRVENTTVYYLVLDVQESDCSVLSRKYWNDCEPPDSRRPSEIVIGQCKVIATRHSHESQDLRVIDFNCTTSSVSSALANTKDSPVLIDFFEDTERYRKQANKALEKYKEENDDFASFRVDRIERVARVRGGEGTGYFVDFSVRNCPRHHFPRHPNVFGFCRADLFYDVEALDLESPKNLVINCEVFDPQEHENINGVPPHLGHPFHWGGHERSSTTKPPFKPHGSRDHHHPHKPHEHGPPPPPDERDHSHGPPLPQGPPPLLPMSCSSCQHATFGTNGAQRHSHNNNSSDLHPHKHHSHEQHPHGHHPHAHHPHEHDTHRQHPHGHHPHGHHPHGHHPHGHHPHGHHPHCHDFQDYGPCDPPPHNQGHCCHGHGPPPGHLRRRGPGKGPRPFHCRQIGSVYRLPPLRKGEVLPLPEANFPSFPLPHHKHPLKPDNQPFPQSVSESCPGKFKSGFPQVSMFFTHTFPK

SEQ ID NO: 43 - protein, 499 aa, Homo sapiens
  misc_feature (181)..(193): heparin binding domain
MDTSPLCFSILLVLCIFIQSSALGQSLKPEPFGRRAQAVETNKTLHEMKTRFLLFGETNQGCQIRINHPDTLQECGFNSSLPLVMIIHGWSVDGVLENWIWQMVAALKSQPAQPVNVGLVDWITLAHDHYTIAVRNTRLVGKEVAALLRWLEESVQLSRSHVHLIGYSLGAHVSGFAGSSIGGTHKIGRITGLDAAGPLFEGSAPSNRLSPDDANFVDAIHTFTREHMGLSVGIKQPIGHYDFYPNGGSFQPGCHFLELYRHIAQHGFNAITQTIKCSHERSVHLFIDSLLHAGTQSMAYPCGDMNSFSQGLCLSCKKGRCNTLGYHVRQEPRSKSKRLFLVTRAQSPFKVYHYQLKIQFINQTETPIQTTFTMSLLGTKEKMQKIPITLGKGIASNKTYSFLITLDVDIGELIMIKFKWENSAVWANVWDTVQTIIPWSTGPRHSGLVLKTIRVKAGETQQRMTFCSENTDDLLLRPTQEKIFVKCEIKSKTSKRKIR

SEQ ID NO: 44 - protein, 475 aa, Homo sapiens
  misc_feature (319)..(331): heparin binding domain
MESKALLVLTLAVWLQSLTASRGGVAAADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSG

SEQ ID NO: 45 - protein, 357 aa, Homo sapiens
MAPRRVRSFLRGLPALLLLLLFLGPWPAASHGGKYSREKNQPKPSPKRESGEEFRMEKLNQLWEKAQRLHLPPVRLAELHADLKIQERDELAWKKLKLDGLDEDGEKEARLIRNLNVILAKYGLDGKKDARQVTSNSLSGTQEDGLDDPRLEKLWHKAKTSGKFSGEELDKLWREFLHHKEKVHEYNVLLETLSRTEEIHENVISPSDLSDIKGSVLHSRHTELKEKLRSINQGLDRLRRVSHQGYSTEAEFEEPRVIDLWDLAQSANLTDKELEAFREELKHFEAKIEKHNHYQKQLEIAHEKLRHAESVGDGERVSRSREKHALLEGRTKELGYTVKKHLQDLSGRISRARHNEL

SEQ ID NO: 46 - protein, 208 aa, Homo sapiens; identical to SEQ ID NO: 22

SEQ ID NO: 47 - protein, 221 aa, Homo sapiens; identical to SEQ ID NO: 15
  misc_feature (193)..(213): heparin binding domain

SEQ ID NO: 48 - protein, 500 aa, Homo sapiens
  misc_feature (325)..(337): heparin binding domain
MSNSVPLLCFWSLCYCFAAGSPVPFGPEGRLEDKLHKPKATQTEVKPSVRFNLRTSKDPEHEGCYLSVGHSQPLEDCSFNMTAKTFFIIHGWTMSGIFENWLHKLVSALHTREKDANVVVVDWLPLAHQLYTDAVNNTRVVGHSIARMLDWLQEKDDFSLGNVHLIGYSLGAHVAGYAGNFVKGTVGRITGLDPAGPMFEGADIHKRLSPDDADFVDVLHTYTRSFGLSIGIQMPVGHIDIYPNGGDFQPGCGLNDVLGSIAYGTITEVVKCEHERAVHLFVDSLVNQDKPSFAFQCTDSNRFKKGICLSCRKNRCNSIGYNAKKMRNKRNSKMYLKTRAGMPFRVYHYQMKIHVFSYKNMGEIEPTFYVTLYGTNADSQTLPLEIVERIEQNATNTFLVYTEEDLGDLLKIQLTWEGASQSWYNLWKEFRSYLSQPRNPGRELNIRRIRVKSGETQRKLTFCTEDPENTSISPGRELWFRKCRDGWRMKNETSPTVELP

SEQ ID NO: 49 - protein, 128 aa, Homo sapiens [sequence omitted]

SEQ ID NO: 50 - protein, 208 aa, Homo sapiens
MWKWILTHCASAFPHLPGCCCCCFLLLFLVSSVPVTCQALGQDMVSPEATNSSSSSFSSPSSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 51 - protein, 208 aa, Homo sapiens; identical to SEQ ID NO: 22

SEQ ID NO: 52 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSIRCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 53 - protein, 357 aa, Homo sapiens; identical to SEQ ID NO: 45

SEQ ID NO: 54 - protein, 317 aa, Homo sapiens [sequence omitted]

SEQ ID NO: 55 - protein, 1404 aa, Homo sapiens
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIWKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTAPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTSDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGEMTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 56 - protein, 499 aa, Homo sapiens
MKHSLNALLIFLIITSAWGGSKGPLDQLEKGGETAQSADPQWEQLNNKNLSMPLLPADFHKENTVTNDWIPEGEEDDDYLDLEKIFSEDDDYIDIVDSLSVSPTDSDVSAGNILQLFHGKSRIQRLNILNAKFAFNLYRVLKDQVNTFDNIFIAPVGISTAMGMISLGLKGETHEQVHSILHFKDFVNASSKYEITTIHNLFRKLTHRLFRRNFGYTLRSVNDLYIQKQFPILLDFKTKVREYYFAEAQIADFSDPAFISKTNNHIMKLTKGLIKDALENIDPATQMMILNCIYFKGSWVNKFPVEMTHNHNFRLNEREVVKVSMMQTKGNFLAANDQELDCDILQLEYVGGISMLIVVPHKMSGMKTLEAQLTPRVVERWQKSMTNRTREVLLPKFKLEKNYNLVESLKLMGIRMLFDKNGNMAGISDQRIAIDLFKHQGTITVNEEGTQATTVTTVGFMPLSTQVRFTVDRPFLFLIYEHRTSCLLFMGRVANPSRS

SEQ ID NO: 57 - protein, 232 aa, Homo sapiens; identical to SEQ ID NO: 5

SEQ ID NO: 58 - protein, 211 aa, Homo sapiens
KDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 59 - protein, 2386 aa, Homo sapiens
  misc_feature (52)..(272): heparin binding domain
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTQGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDPTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNIIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 60 - protein, 662 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKVWNLKAPLVHTPRPGSQECPGDRGQCDEDSRLWPRTGHSPHRLL

SEQ ID NO: 61 - protein, 317 aa, Homo sapiens [sequence omitted]

SEQ ID NO: 62 - DNA, 8815 bp, Homo sapiens
gcccgcgccggctgtgctgcacagggggaggagagggaaccccaggcgcgagcgggaagaggggacctgcagccacaacttctctggtcctctgcatcccttctgtccctccacccgtccccttccccaccctctggcccccaccttcttggaggcgacaacccccgggaggcattagaagggatttttcccgcaggttgcgaagggaagcaaacttggtggcaacttgcctcccggtgcgggcgtctctcccccaccgtctcaacatgcttaggggtccggggcccgggctgctgctgctggccgtccagtgcctggggacagcggtgccctccacgggagcctcgaagagcaagaggcaggctcagcaaatggttcagccccagtccccggtggctgtcagtcaaagcaagcccggttgttatgacaatggaaaacactatcagataaatcaacagtgggagcggacctacctaggcaatgcgttggtttgtacttgttatggaggaagccgaggttttaactgcgagagtaaacctgaagctgaagagacttgctttgacaagtacactgggaacacttaccgagtgggtgacacttatgagcgtcctaaagactccatgatctgggactgtacctgcatcggggctgggcgagggagaataagctgtaccatcgcaaaccgctgccatgaagggggtcagtcctacaagattggtgacacctggaggagaccacatgagactggtggttacatgttagagtgtgtgtgtcttggtaatggaaaaggagaatggacctgcaagcccatagctgagaagtgttttgatcatgctgctgggacttcctatgtggtcggagaaacgtgggagaagccctaccaaggctggatgatggtagattgtacttgcctgggagaaggcagcggacgcatcacttgcacttctagaaatagatgcaacgatcaggacacaaggacatcctatagaattggagacacctggagcaagaaggataatcgaggaaacctgctccagtgcatctgcacaggcaacggccgaggagagtggaagtgtgagaggcacacctctgtgcagaccacatcgagcggatctggccccttcaccgatgttcgtgcagctgtttaccaaccgcagcctcacccccagcctcctccctatggccactgtgtcacagacagtggtgtggtctactctgtggggatgcagtggctgaagacacaaggaaataagcaaatgctttgcacgtgcctgggcaacggagtcagctgccaagagacagctgtaacccagacttacggtggcaactcaaatggagagccatgtgtcttaccattcacctacaatggcaggacgttctactcctgcaccacagaagggcgacaggacggacatctttggtgcagcacaacttcgaattatgagcaggaccagaaatactctttctgcacagaccacactgttttggttcagactcgaggaggaaattccaatggtgccttgtgccacttccccttcctatacaacaaccacaattacactgattgcacttctgagggcagaagagacaacatgaagtggtgtgggaccacacagaactatgatgccgaccagaagtttgggttctgccccatggctgcccacgaggaaatctgcacaaccaatgaaggggtcatgtaccgcattggagatcagtgggataagcagcatgacatgggtcacatgatgaggtgcacgtgtgttgggaatggtcgtggggaatggacatgcattgcctactcgcagcttcgagatcagtgcattgttgatgacatcacttacaatgtgaacgacacattccacaagcgtcatgaagaggggcacatgctgaactgtacatgcttcggtcagggtcggggcaggtggaagtgtgatcccgtcgaccaatgccaggattcagagactgggacgttttatcaaattggagattcatgggagaagtatgtgcatggtgtcagataccagtgctactgctatggccgtggcattggggagtggcattgccaacctttacagacctatccaagctcaagtggtcctgtcgaagtatttatcactgagactccgagtcagcccaactcccaccccatccagtggaatgcaccacagccatctcacatttccaagtacattctcaggtggagacctaaaaattctgtaggccgttggaaggaagctaccataccaggccacttaaactcctacaccatcaaaggcctgaagcctggtgtggtatacgagggccagctcatcagcatccagcagtacggccaccaagaagtgactcgctttgacttcaccaccaccagcaccagcacacctgtgaccagcaacaccgtgacaggagagacgactcccttttctcctcttgtggccacttctgaatctgtgaccgaaatcacagccagtagctttgtggtctcctgggtctcagcttccgacaccgtgtcgggattccgggtggaatatgagctgagtgaggagggagatgagccacagtacctggatcttccaagcacagccacttctgtgaacatccctgacctgcttcctggccgaaaatacattgtaaatgtctatcagatatctgaggatggggagcagagtttgatcctgtctacttcacaaacaacagcgcctgatgcccctcctgacccgactgtggaccaagttgatgacacctcaattgttgttcgctggagcagaccccaggctcccatcacagggtacagaatagtctattcgccatcagtagaaggtagcagcacagaactcaaccttcctgaaactgcaaactccgtcaccctcagtgacttgcaacctggtgttcagtataacatcactatctatgctgtggaagaaaatcaagaaagtacacctgttgtcattcaacaagaaaccactggcaccccacgctcagatacagtgccctctcccagggacctgcagtttgtggaagtgacagacgtgaaggtcaccatcatgtggacaccgcctgagagtgcagtgaccggctaccgtgtggatgtgatccccgtcaacctgcctggcgagcacgggcagaggctgcccatcagcaggaacacctttgcagaagtcaccgggctgtcccctggggtcacctattacttcaaagtctttgcagtgagccatgggagggagagcaagcctctgactgctcaacagacaaccaaactggatgctcccactaacctccagtttgtcaatgaaactgattctactgtcctggtgagatggactccacctcgggcccagataacaggataccgactgaccgtgggccttacccgaagaggacagcccaggcagtacaatgtgggtccctctgtctccaagtacccactgaggaatctgcagcctgcatctgagtacaccgtatccctcgtggccataaagggcaaccaagagagccccaaagccactggagtctttaccacactgcagcctgggagctctattccaccttacaacaccgaggtgactgagaccaccattgtgatcacatggacgcctgctccaagaattggttttaagctgggtgtacgaccaagccagggaggagaggcaccacgagaagtgacttcagactcaggaagcatcgttgtgtccggcttgactccaggagtagaatacgtctacaccatccaagtcctgagagatggacaggaaagagatgcgccaattgtaaacaaagtggtgacaccattgtctccaccaacaaacttgcatctggaggcaaaccctgacactggagtgctcacagtctcctgggagaggagcaccaccccagacattactggttatagaattaccacaacccctacaaacggccagcagggaaattctttggaagaagtggtccatgctgatcagagctcctgcacttttgataacctgagtcccggcctggagtacaatgtcagtgtttacactgtcaaggatgacaaggaaagtgtccctatctctgataccatcatcccagaggtgccccaactcactgacctaagctttgttgatataaccgattcaagcatcggcctgaggtggaccccgctaaactcttccaccattattgggtaccgcatcacagtagttgcggcaggagaaggtatccctatttttgaagattttgtggactcctcagtaggatactacacagtcacagggctggagccgggcattgactatgatatcagcgttatcactctcattaatggcggcgagagtgcccctactacactgacacaacaaacggctgttcctcctcccactgacctgcgattcaccaacattggtccagacaccatgcgtgtcacctgggctccacccccatccattgatttaaccaacttcctggtgcgttactcacctgtgaaaaatgaggaagatgttgcagagttgtcaatttctccttcagacaatgcagtggtcttaacaaatctcctgcctggtacagaatatgtagtgagtgtctccagtgtctacgaacaacatgagagcacacctcttagaggaagacagaaaacaggtcttgattccccaactggcattgacttttctgatattactgccaactcttttactgtgcactggattgctcctcgagccaccatcactggctacaggatccgccatcatcccgagcacttcagtgggagacctcgagaagatcgggtgccccactctcggaattccatcaccctcaccaacctcactccaggcacagagtatgtggtcagcatcgttgctcttaatggcagagaggaaagtcccttattgattggccaacaatcaacagtttctgatgttccgagggacctggaagttgttgctgcgacccccaccagcctactgatcagctgggatgctcctgctgtcacagtgagatattacaggatcacttacggagagacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacagaaattgacaaaccatcccagatgcaagtgaccgatgttcaggacaacagcattagtgtcaagtggctgccttcaagttcccctgttactggttacagagtaaccaccactcccaaaaatggaccaggaccaacaaaaactaaaactgcaggtccagatcaaacagaaatgactattgaaggcttgcagcccacagtggagtatgtggttagtgtctatgctcagaatccaagcggagagagtcagcctctggttcagactgcagtaaccaacattgatcgccctaaaggactggcattcactgatgtggatgtcgattccatcaaaattgcttgggaaagcccacaggggcaagtttccaggtacagggtgacctactcgagccctgaggatggaatccatgagctattccctgcacctgatggtgaagaagacactgcagagctgcaaggcctcagaccgggttctgagtacacagtcagtgtggttgccttgcacgatgatatggagagccagcccctgattggaacccagtccacagctattcctgcaccaactgacctgaagttcactcaggtcacacccacaagcctgagcgcccagtggacaccacccaatgttcagctcactggatatcgagtgcgggtgacccccaaggagaagaccggaccaatgaaagaaatcaaccttgctcctgacagctcatccgtggttgtatcaggacttatggtggccaccaaatatgaagtgagtgtctatgctcttaaggacactttgacaagcagaccagctcagggagttgtcaccactctggagaatgtcagcccaccaagaagggctcgtgtgacagatgctactgagaccaccatcaccattagctggagaaccaagactgagacgatcactggcttccaagttgatgccgttccagccaatggccagactccaatccagagaaccatcaagccagatgtcagaagctacaccatcacaggtttacaaccaggcactgactacaagatctacctgtacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgaggaacatggttttaggcggaccacaccgcccacaacggccacccccataaggcataggccaagaccatacccgccgaatgtaggtgaggaaatccaaattggtcacatccccagggaagatgtagactatcacctgtacccacacggtccgggactcaatccaaatgcctctacaggacaagaagctctctctcagacaaccatctcatgggccccattccaggacacttctgagtacatcatttcatgtcatcctgttggcactgatgaagaacccttacagttcagggttcctggaacttctaccagtgccactctgacaggcctcaccagaggtgccacctacaacatcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttaggctttggaagtggtcatttcagatgtgattcatctagatggtgccatgacaatggtgtgaactacaagattggagagaagtgggaccgtcagggagaaaatggccagatgatgagctgcacatgtcttgggaacggaaaaggagaattcaagtgtgaccctcatgaggcaacgtgttatgatgatgggaagacataccacgtaggagaacagtggcagaaggaatatctcggtgccatttgctcctgcacatgctttggaggccagcggggctggcgctgtgacaactgccgcagacctgggggtgaacccagtcccgaaggcactactggccagtcctacaaccagtattctcagagataccatcagagaacaaacactaatgttaattgcccaattgagtgcttcatgcctttagatgtacaggctgacagagaagattcccgagagtaaatcatctttccaatccagaggaacaagcatgtctctctgccaagatccatctaaactggagtgatgttagcagacccagcttagagttcttctttctttcttaagccctttgctctggaggaagttctccagcttcagctcaactcacagcttctccaagcatcaccctgggagtttcctgagggttttctcataaatgagggctgcacattgcctgttctgcttcgaagtattcaataccgctcagtattttaaatgaagtgattctaagatttggtttgggatcaataggaaagcatatgcagccaaccaagatgcaaatgttttgaaatgatatgaccaaaattttaagtaggaaagtcacccaaacacttctgctttcacttaagtgtctggcccgcaatactgtaggaacaagcatgatcttgttactgtgatattttaaatatccacagtactcactttttccaaatgatcctagtaattgcctagaaatatctttctcttacctgttatttatcaatttttcccagtatttttatacggaaaaaattgtattgaaaacacttagtatgcagttgataagaggaatttggtataattatggtgggtgattattttttatactgtatgtgccaaagctttactactgtggaaagacaactgttttaataaaagatttacattccacaacttgaagttcatctatttgatataagacaccttcgggggaaataattcctgtgaatattctttttcaattcagcaaacatttgaaaatctatgatgtgcaagtctaattgttgatttcagtacaagattttctaaatcagttgctacaaaaactgattggtttttgtcacttcatctcttcactaatggagatagctttacactttctgctttaatagatttaagtggaccccaatatttattaaaattgctagtttaccgttcagaagtataatagaaataatctttagttgctcttttctaaccattgtaattcttcccttcttccctccacctttccttcattgaataaacctctgttcaaagagattgcctgcaagggaaataaaaatgactaagatattaaaaaaaaaaaaaaaaaa

SEQ ID NO: 63 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 64 - protein, 344 aa, Homo sapiens
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 65 - protein, 475 aa, Homo sapiens [sequence omitted]

SEQ ID NO: 66 - protein, 226 aa, Homo sapiens [sequence omitted]

SEQ ID NO: 67 - protein, 497 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 68 - protein, 419 aa, Homo sapiens
  misc_feature (156)..(156): Xaa = any or no amino acid
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPXVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 69 - protein, 180 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRGRNGKKQQRKPLREKMKPERRRPKGRGKRRREKQRPTDCHLCGDAVPRR

(19) United States
(12) Patent Application Publication
(10) Pub. No.: US 2005/0032697 A1
(43) Pub. Date: Feb. 10, 2005

(54) Heparin binding VEGFR-3 ligands

(76) Inventors: Kari Alitalo, Helsinki (FI); Yulong He, Helsinki (FI); Toumas Tammela, Helsinki (FI)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/868,577

(22) Filed: Jun. 14, 2004

Related U.S. Application Data
(63) Continuation-in-part of application No. 10/669,176, filed on Sep. 23, 2003.
(60) Provisional application No. 60/478,114, filed on Jun. 12, 2003.
(60) Provisional application No. 60/478,390, filed on Jun. 12, 2003.

(51) Int. Cl. C07K 14/71; A61K 38/17

(52) U.S. Cl. 514/12; 530/350

(57) ABSTRACT

The present invention is directed to methods and compositions for making and using chimeric polypeptides that comprise a VEGFR-3 ligand and a heparin binding domain. The chimeric molecules of the present invention retain VEGFR-3 binding activity and an enhanced heparin binding activity as compared to native VEGF-C and/or VEGF-D.

### CROSS-REFERENCE TO PRIOR APPLICATIONS

The present application is a continuation-in-part of U.S. patent application Ser. No. 10/669,176 filed Sep. 23, 2003. The present application claims the benefit of priority of U.S. Provisional Patent Application No. 60/478,390, which was filed on filed on Jun. 12, 2003. The present application also claims the benefit of priority of U.S. Patent No.60/478,114, filed Jun. 12, 2003. The entire text of each of the foregoing applications is specifically incorporated herein by reference.

### FIELD OF THE INVENTION

The present application is directed to methods and compositions for promoting lymphangiogenesis and/or angiogenesis. The application describes chimeric polypeptides that comprise a VEGFR-3 ligand and a heparin binding domain and methods and compositions for using the same.

### BACKGROUND

The VEGF proteins and their receptors (VEGFRs) play important roles in both vasculogenesis, the development of the embryonic vasculature from early differentiating endothelial cells, and angiogenesis, the process of forming new blood vessels from pre-existing ones (Risau et al., Dev Biol 125:441-450 (1988); Zachary, Intl J Biochem Cell Bio 30:1169-1174, 1998; Neufeld et al., FASEB J 13:9-22, 1999); Ferrara, J Mol Med 77:527-543, 1999). Both processes depend on tight control of endothelial cell proliferation, migration, differentiation, and survival. Dysfunction of the endothelial cell regulatory system is a key feature of cancer and several diseases associated with abnormal angiogenesis, such as proliferative retinopathies, age-related muscular degeneration, rheumatoid arthritis, and psoriasis. Understanding of the specific biological function of the key players involved in regulating endothelial cells will lead to more effective therapeutic applications to treat such diseases (Zachary, Intl J Biochem Cell Bio 30:1169-1174, 1998; Neufeld et al., FASEB J 13:9-22, 1999; Ferrara, J Mol Med 77:527-543, 1999).

The mammalian vascular endothelial growth factor (VEGF) family members identified to date, including VEGF, VEGF-B, VEGF-C, VEGF-D, and placenta growth factor (PIGF), play crucial roles in the physiological and pathological regulation of vasculogenesis, hematopoiesis, angiogenesis, lymphangiogenesis, and vascular permeability (Ferrara and Davis-Smyth, Endocr Rev 18: 4-25, 1997; Veikkola et al., Cancer Res 60: 203-12, 2000; Carmeliet and Jain, Nature, 407:249-57, 2000). VEGF, also identified as a potent vascular permeability-enhancing factor (Dvorak, et al., Am J Pathol 146: 1029-39, 1995), is a potent growth factor for blood vessel formation and plays an essential role in this process (Ferrara and Davis-Smyth, Endocr Rev 18: 4-25, 1997].

It has been noted that both insufficient and excessive VEGF lead to abnormal blood vessel formation. This property and the permeability-inducing property of VEGF may pose difficulties for its in vivo application. Both VEGF-C and VEGF-D have been shown to induce lymphangiogenesis in transgenic mice and in other in vivo models (Jeltsch et al., Science 276:1423-5, 1997; Oh et al., Dev Biol 188: 96-109, 1997; Veikkola et al., EMBO J 20: 1223-31, 2001). VEGF-C and VEGF-D signal primarily through VEGFR-3 [Veikkola et al., EMBO J 20: 1223-31, 2001; Joukov et al., EMBO J 15: 290-98 1996; Lee et al., Proc Natl Acad Sci USA 93: 1988-92, 1996; Achen et al., Proc Natl Acad Sci USA 95: 548-53, 1998; Makinen et al., Nat Med 7: 199-205, 2001].

VEGF-C and VEGF-D are produced as precursor proteins with N- and C-terminal pro-peptides flanking the VEGF homology domain (VHD; Joukov et al., EMBO J. 16:3898-3011, 1997). A schematic view of the VEGF-C prepro-peptide is shown in FIG. 1A. The proteolytic processing of prepro-polypeptides of VEGF-C and VEGF-D increases their affinities for VEGFR-3, and the fully-processed mature forms can also bind to, and activate, VEGFR-2 (Joukov et al., EMBO J 16: 3898-911, 1997; Stacker et al., J Biol Chem 274 32127-36, 1999). Both factors can thus theoretically exert angiogenic activity via VEGFR-2.

However, in transgenic models in which both wild type and mutant forms of VEGF-C induced lymphangiogenesis in the skin, no angiogenic effect was observed. It was therefore suggested that during embryonic development VEGF-C may not be fully processed to a form that activates the VEGFR-2 of blood vessels [Jeltsch et al., Science 276:1423-5, 1997; Veikkola et al., Embo J 20: 1223-31, 2001]. In addition, although the recombinant mature form of VEGF-C has been shown to induce angiogenesis and lymphangiogenesis (Cao et al. Proc Natl Acad Sci USA 95: 14389-94, 1998; Marconcini et al., Proc Natl Acad Sci USA 96: 9671-6, 1999), its angiogenic activity was weak when it was delivered through viral vectors such as adenoviral or adeno-associated viral vector.

Thus, while VEGF-C and VEGF-D have been shown to have significant angiogenic and lymphangiogenic effects in a number of settings, there remains a desire to enhance the angiogenic and/or lymphangiogenic effects of these molecules to render them more efficacious in these indications.

### SUMMARY OF THE INVENTION

The present invention addresses the need for more efficacious angiogenic and lymphangiogenic VEGF molecules by providing chimeric polypeptides that comprise a VEGF homology domain (VHD) and a heparin binding domain.

The invention includes numerous aspects and embodiments described throughout the application. In certain exemplary embodiments, the present invention is a compound comprising the formula X-B-Z or Z-B-X, wherein X binds Vascular Endothelial Growth Factor Receptor 3 (VEGFR-3) and comprises an amino acid sequence at least 70%, or more preferably 75%, 80%, 85%, 90%, 95%, 96%, 97%, 98%, 99%, or 100% identical to a VEGFR-3 ligand selected from the group consisting of:

(a) the prepro-VEGF-C amino acid sequence set forth in SEQ ID NO: 2;

(b) fragments of (a) that bind VEGFR-3;

(c) the prepro-VEGF-D amino acid sequence set forth in SEQ ID NO: 4; and

(d) fragments of (c) that bind VEGFR-3;

wherein Z comprises a heparin-binding amino acid sequence; and

wherein B comprises a covalent attachment linking X to Z.

In particular embodiments, it is contemplated that the compound comprises the formula X-B-Z or Z-B-X, wherein X binds Vascular Endothelial Growth Factor Receptor 3 (VEGFR-3) and comprises an amino acid sequence at least 70%, or more preferably 75%, 80%, 85%, 90%, 95%, 96%, 97%, 98%, 99%, or 100% identical to a VEGFR-3 ligand that is a chimera comprised of sequences of two or more vacular endothelial growth factors, including the chimeras described in WO 01/62942; wherein Z comprises a heparin-binding amino acid sequence, with the proviso that the heparin binding sequence is not identical to a VEGF-A heparin binding sequence; and wherein B comprises a covalent attachment linking X to Z.

The compounds described above may also preferably bind Vascular Endothelial Growth Factor Receptor 2 (VEGFR-2). The compounds of the invention advantageously comprise a heparin binding domain that facilitates the biological bioavailability of the growth factor. As such, in the chimeric compounds of the invention the moiety Z may be any heparin binding domain that retains an heparin binding activity when joined to a growth factor as described herein. In specific embodiments, the heparin binding amino acid sequence is derived from a vascular endothelial growth factor. In exemplary embodiments, the sequence comprises an amino acid sequence at least 70% identical, or more preferably at least 75%, 80%, 85%, 90%, 95%, 96%, 97%, 98%, 99%, or 100% identical, to a sequence selected from the group consisting of:

(a) amino acids 142-165 of the VEGF₂₀₆ (amino acids encoded by exon 6a of VEGF);

(b) amino acids 183 to 226 of the VEGF₂₀₆ (amino acids encoded by exon 7 of VEGF);

(c) amino acids 142-165 joined directly to amino acids 183-226 of the VEGF₂₀₆ (amino acids encoded by exons 6 through 7 of VEGF);

(d) amino acids 142 to 226 of the VEGF₂₀₆ (amino acids encoded by exons 6 though 8 of VEGF);

(e) amino acids 138 to 182 of the VEGF-B₁₆₇ sequence set forth in SEQ ID NO: 8;

(f) amino acids 193 to 213 of the PlGF-3 sequence set forth in SEQ ID NO: 15;

(g) amino acids of 142 to 162 of the PlGF-2 sequence set forth in SEQ ID NO: 69;

(h) fragments of (a)-(g) that bind heparin.

Additional exemplary embodiments comprise a sequence at least 70%, 75%, 80%, 85%, 90%, 95%, 96%, 97%, 98%, 99%, or 100% identical to the heparin binding regions of many additional sequences identified in the detailed description. Human sequences are preferred for molecules to be administered to humans.

In particularly preferred embodiments, the moiety defined by the formula X-B-Z or Z-B-X, defines a polypeptide. Preferably, such a polypeptide further comprises a signal peptide at the amino terminus of the polypeptide, wherein the signal peptide directs secretion of a polypeptide comprising X-B-Z or Z-B-X from a cell that expresses the polypeptide.

In the compositions described herein, the moiety B is a linking moiety that links X and Z. Such a moiety may be a peptide bond or alternatively may be a peptide or other organic linker commonly used in the preparation of chimeric molecules. The peptide linker may, for example be an amino acid linker up to 500 amino acids. In other specific embodiments, B comprises a peptide bond that is cleavable by an agent that fails to cleave the amino acid sequence X that binds VEGFR-3. More particularly, such a peptide bond is preferably cleaved by a protease. In exemplary specific embodiments, the moiety B comprises an amino acid sequence that contains a protease cleavage site selected from the group consisting of a Factor Xa cleavage site, an enterokinase cleavage site, a thrombin cleavage site, a TEV cleavage site, and a PreScission cleavage site. In other preferred embodiments, the moiety B comprises an amino acid sequence of at least four, and more preferably at least six amino acids from a VEGF-C or VEGF-D amino acid sequence, wherein the at least four amino acids are cleaved in vivo to separate an amino-terminal VEGF-C or VEGF-D propeptide from a mature VEGF-C or VEGF-D protein.

It is particularly contemplated that in the compounds of the invention X comprises an amino acid sequence at least 95% identical to the prepro-VEGF-C amino acid sequence set forth in SEQ ID NO: 2 or to a fragment thereof that binds VEGFR-3. Other embodiments contemplate that X comprises an amino acid sequence at least 95% identical to the prepro-VEGF-C amino acid sequence set forth in SEQ ID NO: 2 or to a fragment thereof that binds VEGFR-3, with the proviso that the cysteine corresponding to amino acid position 156 of SEQ ID NO: 2 has been deleted or replaced with an amino acid other than cysteine, and the resultant amino acid sequence binds VEGFR-3 but has reduced VEGFR-2 binding. Such VEGF-C ΔC₁₅₆ polypeptides are described in detail in International Patent Publication No. WO 98/33917, incorporated herein by reference.

In still other examples of the compounds useful in the present invention, X comprises an amino acid sequence identical to the prepro-VEGF-C amino acid sequence set forth in SEQ ID NO: 2 or to a fragment thereof that binds VEGFR-3. In yet further embodiments, X may comprise an amino acid sequence identical to the prepro-VEGF-C amino acid sequence set forth in SEQ ID NO: 2 or to a fragment thereof that binds VEGFR-3, with the proviso that the cysteine corresponding to amino acid position 156 of SEQ ID NO: 2 has been deleted or replaced with an amino acid other than cysteine, and the resultant amino acid sequence binds VEGFR-3 but has reduced VEGFR-2 binding.

In alternative embodiments, X may comprise an amino acid sequence at least 95% identical to the prepro-VEGF-D amino acid sequence set forth in SEQ ID NO: 4 or to a fragment thereof that binds VEGFR-3. Other specific embodiments contemplate that X comprises an amino acid sequence identical to the prepro-VEGF-D amino acid sequence set forth in SEQ ID NO: 4 or to a fragment thereof that binds VEGFR-3.

It is particularly contemplated that any of the compounds of the invention may be prepared to further include a peptide tag, e.g., a polyhistidine tag. Inclusion of such a tag may facilitate purification. In additional embodiments, the compounds may be PEGylated with one or more polyethylene glycol (PEG) moieties.

The compounds of the present invention may advantageously be formulated into compositions wherein such compositions comprise a compound of the invention in a pharmaceutically acceptable carrier. The compounds of the invention are preferably useful in the manufacture of medicaments. For example, the compounds of the invention have a use in the manufacture of a medicament for modulation of VEGFR-3 and/or VEGFR-2 to treat diseases or conditions that would benefit from such modulation.

Other compositions of the present invention describe polynucleotides that comprising a nucleotide sequence that encodes a chimeric protein compound of formula X-B-Z or Z-B-X as discussed above and described in further detail in the description below. In specific embodiments, the polynucleotide further comprises a nucleotide sequence that encodes a signal peptide fused in-frame with the polypeptides described above. Vectors that comprise such polynucleotides also are contemplated. The present invention particularly contemplates an expression vector comprising a polynucleotide comprising a nucleotide sequence that encodes a chimeric protein compound of formula X-B-Z or Z-B-X operably linked to an expression control sequence. In certain embodiments, the expression control sequence is an endothelial cell specific promoter. The expression vector may be any vector used for the expression of a nucleic acid and may for example, be selected from the group consisting of replication deficient adenoviral vectors, adeno-associated viral vectors, and lentivirus vectors. The polynucleotides and vectors of the invention may be formulated as a compositions in which the polynucleotide or the vector is presented in a pharmaceutically acceptable carrier. The polynucleotides or vectors according to the invention may be used in the manufacture of a medicament for modulation of VEGFR-3 and/or VEGFR-2, to treat diseases or conditions that would benefit from such modulation.

Also contemplated are host cells that have been transformed or transfected with a polynucleotide or vector of the invention.

Other aspects of the invention are directed to methods of modulating the growth of mammalian endothelial cells or mammalian endothelial precursor cells, comprising contacting the cells with a composition comprising a member selected from the group consisting of a polypeptide compound of formula X-B-Z or Z-B-X; a polynucleotide that encodes such a compound; an expression vector containing such a polynucleotide operatively linked to an expression control sequence; and a cell transformed or transfected with such a polynucleotide or such a vector that expresses the polypeptide compound of formula X-B-Z or Z-B-X. In certain embodiments, the contacting comprises administering the composition to a mammalian subject in an amount effective to modulate endothelial cell growth in vivo. In particular embodiments, the mammalian subject is a human.

Also contemplated herein is a method of modulating growth of mammalian hematopoietic progenitor cells, comprising contacting the cells with a composition comprising a member selected from the group consisting of polypeptide compound of formula X-B-Z or Z-B-X; a polynucleotide that encodes such a compound; an expression vector containing such a polynucleotide operatively linked to an expression control sequence; and a cell transformed or transfected with such a polynucleotide or such a vector that expresses the polypeptide compound of formula X-B-Z or Z-B-X.

The methods described herein may be used for the activation of VEGFR-3. Such methods would generally comprise contacting cells that express VEGFR-3 with a composition comprising a polypeptide compound of formula X-B-Z or Z-B-X.

Other embodiments of the invention are directed to methods of stimulating lymphangiogenesis in a mammal comprising contacting said mammal with, and/or administering to said mammal, a composition comprising a member selected from the group consisting of polypeptide compound of formula X-B-Z or Z-B-X; a polynucleotide that encodes such a compound; an expression vector containing such a polynucleotide operatively linked to an expression control sequence; and a cell transformed or transfected with such a polynucleotide or such a vector that expresses the polypeptide compound of formula X-B-Z or Z-B-X.

Also contemplated are methods of stimulating angiogenesis in a mammal comprising contacting said mammal with a composition comprising a member selected from the group consisting of

(a) a polypeptide compound of formula X-B-Z or Z-B-X wherein the compound binds Vascular Endothelial Growth Factor Receptor 2 (VEGFR-2); a polynucleotide that encodes such a polypeptide compound, an expression vector containing such a polynucleotide operatively linked to an expression control sequence; and a cell transformed or transfected with such a polynucleotide or vectors that expresses the polypeptide compound of formula X-B-Z or Z-B-X wherein the compound binds Vascular Endothelial Growth Factor Receptor 2 (VEGFR-2).

It has been shown that VEGF-C functions as a neurotrophic and neuroprotective growth factor (for detailed description see U.S. patent application Ser. No. 10/669,176, filed Sep. 23, 2003, incorporated herein by reference in its entirety). As such, the compositions of the present invention may be used alone or in combination with additional agents to treat disorders in which neuronal loss or functional deficiency is a problem.

In specific embodiments, the disease or disorder being treated is a neurodegenerative disorder, wherein the neurodegenerative disorder is selected from the group consisting of Alzheimer's disease, Parkinson's disease, Huntington's disease, motor neuron disease, Amyotrophic Lateral Sclerosis (ALS), dementia and cerebral palsy. In another embodiment, the disease or condition is selected from the group consisting of neural trauma or neural injury. Methods of the invention also can be performed to treat or ameliorate the effects of neural trauma or injury, such as injury related to stroke, spinal cord injury, post-operative injury, brain ischemia and other traumas. Patients affected by any of the above disorders are administered a polypeptide of formula X-B-Z or Z-B-X, either systemically, or preferably at the site of neuropathology, to stimulate the proliferation of neural stem cells in vivo. As described above for other indications, administration of polynucleotides, expression vectors, and transformed cells is specifically contemplated for neurological indications. Alternatively, patients are administered neural stem cells isolated from a biological sample, from a commercial source or an immortalized neural stem cell, which have been transformed to express a polypeptide of formula X-B-Z or Z-B-X. The neural stem cells are then administered to a patient with a neurodegenerative disorder or neural trauma such that they will migrate to the site of neural degeneration and proliferate. In a related variation, neuronal stem cells are cultured ex vivo with polypeptides of the invention before administration.

Thus, an aspect of the invention is a method of promoting recruitment, proliferation, differentiation, migration or survival of neuronal cells or neuronal precursor cells in a mammalian subject comprising administering to the subject a composition comprising a vascular endothelial growth factor C (VEGF-C) product or a vascular endothelial growth factor D (VEGF-D) product, wherein the VEGF-C or VEGF-D product is a heparin binding polypeptide as described herein, or a polynucleotide that encodes such a polypeptide, or vector comprising such a polynucleotide, or a host cell that expresses such a polypeptide. In preferred variations, the method further comprises a step, prior to the administrating step, of identifying a mammalian subject in need of neuronal cell or neuronal precursor cell recruitment, proliferation, or differentiation. Candidates include subjects having disorders described in the preceding paragraph.

Combination therapy is specifically contemplated for neurological therapies, such as co-administration of the VEGF-C or VEGF-D product in conjunction with a neural growth factor. Exemplary factors include interferon gamma, nerve growth factor, epidermal growth factor (EGF), basic fibroblast growth factor (bFGF), neurogenin, brain derived neurotrophic factor (BDNF), thyroid hormone, bone morphogenic proteins (BMPs), leukemia inhibitory factor (LIF), sonic hedgehog, glial cell line-derived neurotrophic factor (GDNFs), vascular endothelial growth factor (VEGF), interleukins, interferons, stem cell factor (SCF), activins, inhibins, chemokines, retinoic acid and ciliary neurotrophic factor (CNTF).

Compositions comprising polypeptides of the invention and any of the foregoing polypeptides, or comprising one or more polynucleotides that encode polypeptides of the invention and any of the foregoing polypeptides, are specifically contemplated as an aspect of the invention.

In yet another aspect, the invention provides compositions and methods of treatment involving polypeptides of the invention (e.g., polypeptides of formula X-B-Z or Z-B-X, or polynucleotides encoding them) in combination with other polypeptides that will enhance vessel formation or integrity, are specifically contemplated. The polypeptides (or encoding polynucleotides) specifically contemplated include, but are not limited to, Angiopoietin-1 (Ang-1, SEQ ID NO: 67), PDGF-A, PDGF-B, PDGF-C, PDGF-D, VEGF, VEGF-B, and combinations thereof. Such combinations will be useful in the optimal induction of functional vessels, such as lymphatic vessels.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. Where protein therapy is described, embodiments involving polynucleotide therapy (using polynucleotides that encode the protein) are specifically contemplated, and the reverse also is true. Where embodiments of the invention are described with respect to VEGF-C, it should be appreciated that analogous embodiments involving VEGF-D are specifically contemplated.

In addition to the foregoing, the invention includes, as an additional aspect, all embodiments of the invention narrower in scope in any way than the variations defined by specific paragraphs above. For example, certain aspects of the invention that are described as a genus, and it should be understood that every member of a genus is, individually, an aspect of the invention. Also, aspects described as a genus or selecting a member of a genus, should be understood to embrace combinations of two or more members of the genus. Although the applicant(s) invented the fill scope of the invention described herein, the applicants do not intend to claim subject matter described in the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

The following drawings form part of the present specification and are included to further illustrate aspects of the present invention. The invention may be better understood by reference to the drawings in combination with the detailed description of the specific embodiments presented herein.

FIG. 1A schematically depicts the proteolytic processing of VEGF-C (Joukov et al., EMBO J 16: 3898-911, 1997). SS, signal sequence; N-term and C-term, N-terminal and C-terminal (silk homology domain) propeptides; VHD, VEGF homology domain; arrowheads, cleavage sites; and disulfide bonds are marked as -S-S- and dotted lines as non-covalent bonds.

FIG. 1B schematically depicts VEGF splice variants (named VEGF₁₂₁, VEGF₁₄₅, VEGF₁₆₅, VEGF₁₈₉, and VEGF₂₀₆) generated by alternative splicing of the eight exons (numbered 1 to 8 shown at the bottom) of the human VEGF gene.

FIG. 1C is a schematic illustration of two VEGF-C/VEGF chimeric molecules comprised of the signal sequence and the VEGF homology domain of VEGF-C, and VEGF exon 6-8 or exon 7-8 encoded sequences (CA89 and CA65, respectively).

FIG. 1D is an autoradiogram depicting immunoprecipitation analysis of radiolabeled, secreted proteins in the conditioned medium from the 293T cells transfected with pEBS7/CA89 (with or without heparin 20 unit/ml incuded in the medium), pEBS7/CA65 or the pEBS7 vector alone.

FIG. 2 is a graph depicting absorbance measurements (540 nm wavelength) of reaction products in a cell viability assay to measure biological activity of the chimeric molecules depicted in FIG. 1C-1D. The biological activity of the VEGF-C chimeric proteins was demonstrated by a bioassay using Ba/F3 cells expressing a chimeric VEGFR-3/erythropoietin (Epo) receptor which transmitted survival and proliferation signals of VEGF-C for the IL-3 dependent Ba/F3/VEGFR-3 cells. Data represent the mean values from triplicate assays.

FIG. 3A. Immunoprecipitation and polyacrylamide gel electrophoresis of secreted proteins (labeled with ³⁵S) from the conditioned medium of 293T cells transfected with pEBS7/CA89 (CA89), pEBS7/CA65 (CA65), pEBS7/VEGF-C N C (N C), or the pEBS7 vector, with neuropilin-1-Ig (NP1) and neuropilin-2-Ig (NP2)

FIG. 3B. Immunoprecipitation and polyacrylamide gel electrophoresis of secreted proteins (labeled with ³⁵S) from the conditioned medium of 293T cells transfected with pEBS7/CA89 (CA89), pEBS7/CA65 (CA65), pEBS7VEGF-CΔNΔC (NΔC), or the pEBS7 vector, with VEGFR-1-Ig (R-1), VEGFR-2-Ig (R-2) and VEGFR-3-Ig (R-3).

FIG. 4A. Analysis of viral expression of the chimeric molecules. Recombinant AAV (A) expression of CA89, CA65, VEGF-CΔNΔC and VEGF-C were analysed by immunoprecipitation of metabolically labelled proteins with anti-VEGF-C serum followed by SDS-PAGE under reducing conditions.

FIG. 4B. Analysis of viral expression of the chimeric molecules. Recombinant adenoviral expression of CA89, CA65, VEGF-CΔNΔC and VEGF-C were analysed by immunoprecipitation of metabolically labelled proteins with anti-VEGF-C serum followed by SDS-PAGE under reducing conditions.

### DETAILED DESCRIPTION OF THE PREFERRED EMBODIMENTS

VEGF-C and VEGF-D are ligands for Flt4 receptor tyrosine kinase, also known as Vascular Endothelial Growth Factor Receptor 3 (VEGFR-3). VEGFR-3 is primarily present on lymphatic endothelia and through interaction with this receptor, these factors are thought to mediate lymphangiogenesis. Angiogenic effects of VEGF-C and VEGF-D are thought to be mediated through VEGFR-2. However, mature forms of VEGF-C delivered by means such as adenoviral gene therapy vectors induced only weak lymphangiongiogenic activity and little angiogenic activity, if any, in mice.

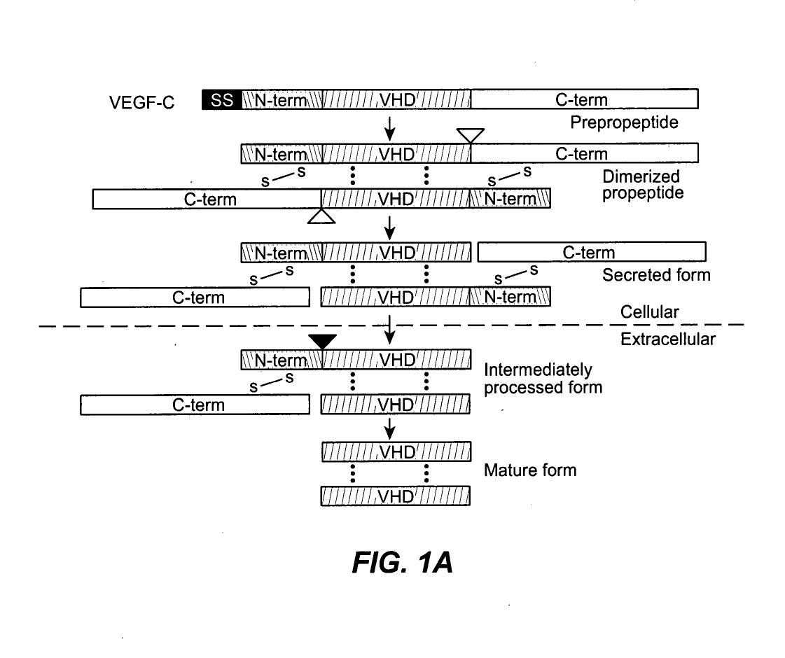

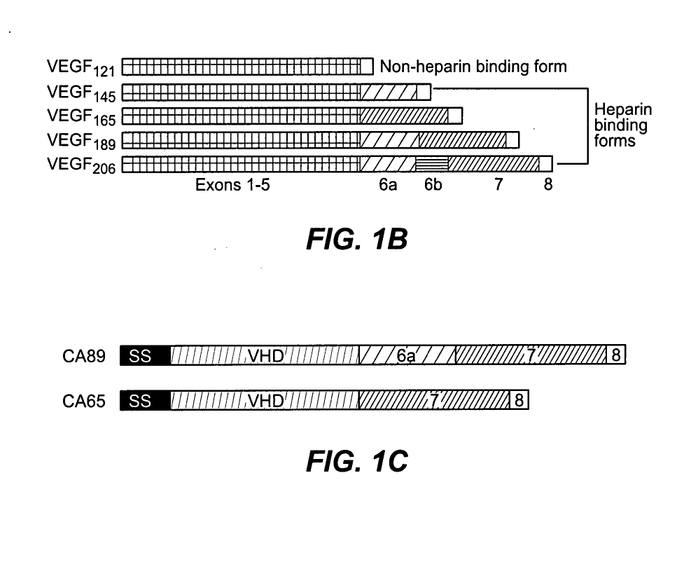

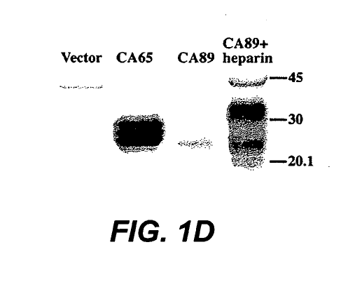

This weak activity suggests that the concentration of the protein present may not be sufficient, or that the half-life of the mature form of VEGF-C protein may be too short, to induce a potent angiogenic effect. VEGF, which has potent angiogenic activity, includes a heparin binding domain. VEGF₁₂₁ has potent angiogenic activity, but does not contain a heparin binding domain. The major forms of VEGF are VEGF₁₂₁, VEGF₁₄₅, VEGF₁₆₅, VEGF₁₈₉ and VEGF₂₀₆, which result from alternative RNA splicing (FIG. 1B) (Ferrara and Davis-Smyth, Endocr Rev 18: 4-25, 1997). An important biological property that distinguishes these VEGF isoforms from each other is their different binding affinities to heparin and heparan sulfate. The four longer isoforms described above contain a heparin binding domain encoded by exon 6 and/or exon 7. The 21 amino acids encoded by exon 6 contain a heparin binding domain and also elements that enable binding to extracellular matrix (Poltorak et al., J. Biol. Chem. 272:7151-8, 1997). Molecules containing the cationic polypeptide sequence encoded by exon 7 (44 amino acids) are also heparin-binding and remain bound to the cell surface and the extracellular matrix. Recently, it has been shown that carboxymethyl benzylamide dextran, a heparin-like molecule, effectively inhibits the activity of VEGF₁₆₅ by interfering with heparin binding to VEGF₁₆₅ (Hamma-Kourbali et al., J Biol Chem., 276(43):39748-54, 2001). There is also other evidence that points to the importance of the heparin binding property of growth factors for their biological activities (Dougher et al., Growth Factors, 14: 257-68, 1997; Carmeliet et al., Nat Med 5: 495-502, 1999; Ruhrberg et al., Genes Dev 16 2684-98, 2002).

VEGF-C and VEGF-D do not have significant heparin binding activity (and, for the purposes of this invention, are not “heparin binding” as that term is used). In order to achieve maximum activation of VEGFR-2 and VEGFR-3 in vivo, and produce VEGF-C and/or VEGF-D molecules that are more potent in inducing angiogenesis and/or lymphangiogenesis, the inventors have produced or described chimeric molecules of VEGF-C and VEGF-D in which the VHD domain is fused or otherwise linked to a heparin binding domain. Methods and compositions for making and using these molecules are described in further detail herein below.

A. Chimeric Molecules of the Present Invention

The present invention provides chimeric VEGFR-3 ligands of the formula X-B-Z or Z-B-X, where domain X binds Vascular Endothelial Growth Factor Receptor 3 (VEGFR-3) and domain Z comprises a heparin binding amino acid sequence. “Domain” B, which comprises a covalent attachment linking X to Z, and at its simplest, is nothing more than a peptide bond or other covalent bond Preferably, domain X comprises an amino acid sequence at least 90% identical to a prepro-VEGF-C amino acid sequence, a fragment of VEGF-C that possesses VEGFR3 binding activity, a prepro-VEGF-D amino acid sequence, or a fragment of VEGF-D that possesses VEGFR3 binding activity. These and other molecules that may serve as X are described in further detail herein.

The chimeric molecules of the present invention are engineered to possess a heparin binding domain Z which preferably increases potency of the molecule as an inducer of angiogenesis and/or lymphangiogenesis, as compared to a similar VEGFR-3 ligand that lacks a heparin binding domain (such as wildtype VEGF-C or -D). This increase in potency may, for example, be due to an increase in the half-life of the chimeric molecule in vivo as compared to the unmodified VEGFR-3 ligand, or to better or more sustained localization in the bloodstream, lymph, or vessel tissues, or other tisses.

a. Domain X: a VEGFR-3 Binding Domain

The VEGFR-3 ligand binding domain of molecules of the invention can be any amino acid sequence that binds VEGFR-3, and confers VEGFR-3 binding to the molecules of the invention. For the purposes of the invention, VEGFR-3 binding means binding to the extracellular domain of human VEGFR-3 (Flt4 receptor tyrosine kinase) as described in U.S. Pat. No. 5,776,755, incorporated herein by reference. Molecules that have at least 10% of the binding affinity of fully-processed (mature) human VEGF-C or VEGF-D for VEGFR-3 are considered molecules that bind VEGFR-3.

Preferred VEGFR-3 binding domains share significant amino acid similarity to a naturally occurring vertebrate VEGF-C or VEGF-D, many of which have been described in the literature and others of which can be cloned from genomic DNA or cDNA libraries, and using PCR and/or standard hybridization techniques and using known VEGF-C or -D cDNAs as probes. For example, preferred molecules have at least 70% amino acid identity to a naturally occurring VEGF-C or -D protein or to a fragment thereof that binds VEGFR-3. Still more preferred are VEGFR-3 binding domains with at least 75%, 80%, 85%, 90%, 95%, 96%, 97%, 98%, or 99% amino acid sequence identity with the natural/wild type vertebrate VEGFR-3 ligand sequence. Descriptions herein of embodiments involving wild type sequences should be understood also to apply to variants sharing such amino acid similarity. It will be appreciated that conservative substitutions and/or substitutions based on sequence alignments with species homologues are less likely to diminish VEGFR-3 binding activity compared to the wild type reference sequence.

A very highly preferred wild type VEGFR-3 ligand for use as the VEGFR-3 binding domain is human prepro-VEGF-C and VEGFR-3 binding fragments thereof. Human VEGF-C polypeptides that may be used as domain X are described in WO 97/05250, WO 98/33917, WO 00/24412, and U.S. Pat. Nos. 6,221,839, 6,361,946, 6,645,933, 6,730,658 and 6,245,530, each of which is incorporated herein by reference in its entirety.

VEGF-C comprises a VHD that is approximately 30% identical at the amino acid level to VEGF. VEGF-C is originally expressed as a larger precursor protein, prepro-VEGF-C, having extensive amino- and carboxy-terminal peptide sequences flanking the VHD, with the C-terminal peptide containing tandemly repeated cysteine residues in a motif typical of Balbiani ring 3 protein. The nucleic acid and amino acid sequences of human prepro-VEGF-C are set forth in SEQ ID NO:1 and SEQ ID NO:2, respectively. Prepro-VEGF-C undergoes extensive proteolytic maturation involving the successive cleavage of a signal peptide, the C-terminal pro-peptide, and the N-terminal pro-peptide, as described in Joukov et al. (EMBO J., 16:(13):3898-3911, 1997) and in the above-referenced patents. Secreted VEGF-C protein consists of a non-covalently linked homodimer, in which each monomer contains the VHD. The intermediate forms of VEGF-C produced by partial proteolytic processing show increasing affinity for the VEGFR-3 receptor, and the mature protein is also able to bind to the VEGFR-2 receptor. (Joukov et al., EMBO J., 16:(13):3898-3911, 1997). The entire text of U.S. Pat. No. 6,361,946 is incorporated herein by reference as providing a teaching of the sequence of the VEGF-C protein, gene and mutants thereof.

For treatment of humans, VEGF-C polypeptides with an amino acid sequence of a human VEGF-C are highly preferred, and polynucleotides comprising a nucleotide sequence of a human VEGF-C cDNA are highly preferred. By “human VEGF-C” is meant a polypeptide corresponding to a naturally occurring protein (prepro-protein, partially-processed protein, or fully-processed mature protein) encoded by any allele of the human VEGF-C gene, or a polypeptide comprising a biologically active fragment of a naturally-occurring mature protein. By way of example, a human VEGF-C comprises a continuous portion of the amino acid sequence set forth in SEQ ID NO: 2 sufficient to permit the polypeptide to bind VEGFR-3 in cells that express VEGFR-3. A polypeptide comprising amino acids 131-211 of SEQ ID NO: 2 is specifically contemplated. For example, polypeptides having an amino acid sequence comprising a continuous portion of SEQ ID NO: 2, the continuous portion having, as its amino terminus, an amino acid selected from the group consisting of positions 30-131 of SEQ ID NO: 2, and having, as its carboxyl terminus, an amino acid selected from the group consisting of positions 211-419 of SEQ ID NO: 2 are contemplated. As explained elsewhere herein in greater detail, VEGF-C biological activities, especially those mediated through VEGFR-2, increase upon processing of both an amino-terminal and carboxyl-terminal pro-peptide. Thus, an amino terminus selected from the group consisting of positions 102-131 of SEQ ID NO: 2 is preferred, and an amino terminus selected from the group consisting of positions 103-113 of SEQ ID NO: 2 is highly preferred. Likewise, a carboxyl terminus selected from the group consisting of positions 211-227 of SEQ ID NO: 2 is preferred. As stated above, the term “human VEGF-C” also is intended to encompass polypeptides encoded by allelic variants of the human VEGF-C characterized by the sequences set forth in SEQ ID NOs: 1 & 2.

Moreover, since the therapeutic VEGF-C is to be administered as recombinant VEGF-C or indirectly via somatic gene therapy, it is within the skill in the art (and an aspect of the invention) to make and use analogs of human VEGF-C (and polynucleotides that encode such analogs) wherein one or more amino acids have been added, deleted, or replaced with other amino acids, especially with conservative replacements, and wherein the VEGFR-3 binding activity has been retained. Analogs that retain VEGFR-3 binding biological activity are contemplated as VEGF-C polypeptides for use in the present invention. In a preferred embodiment, analogs having 1, 2, 3, 4, 5, 6, 7, 8, 9, 10, 11, 12, 13, 14, 15, 16, 17, 18, 19, 20, 21, 22, 23, 24, or 25 such modifications and that retain VEGFR-3 binding activity are contemplated as VEGF-C polypeptides for use in the present invention. Polynucleotides encoding such analogs are generated using conventional PCR, site-directed mutagenesis, and chemical synthesis techniques. Molecules that bind and stimulate phosphorylation of VEGFR-3 are preferred.

Conservative substitutions include the replacement of an amino acid by a residue having similar physicochemical properties, such as substituting one aliphatic residue (Ile, Val, Leu or Ala) for another, or substitution between basic residues Lys and Arg, acidic residues Glu and Asp, amide residues Gln and Asn, hydroxyl residues Ser and Tyr, or aromatic residues Phe and Tyr. Further information regarding making phenotypically silent amino acid exchanges may be found in Bowie et al., Science 247:1306 1310 (1990).

In another variation, the VEGR-3 binding domain has an amino acid sequence similar to or identical to a mutant VEGF-C, in which a single cysteine (at position 156 of the human prepro-VEGF-C sequence) is either substituted by another amino acid or deleted (SEQ ID NO: 68). Such VEGF-CΔCys₁₅₆ (SEQ ID NO: 68) mutants, even when fully processed by removal of both pro-peptides, fail to bind VEGFR-2 but remain capable of binding and activating VEGFR-3. Such polypeptides are described in International Patent Publication No. WO 98/33917 and U.S. Pat. Nos. 6,130,071, and 6,361,946, each of which is incorporated herein by reference in its entirety, especially for their teachings of VEGF-C ΔCys₁₅₆ molecules which may be used in producing chimeras of the present invention which comprise VEGF-C ΔCys₁₅₆ as subunit X of the chimera.

Another highly preferred wild type VEGFR-3 ligand for use in constructing chimeric molecules of the invention is human VEGF-D. VEGF-D is initially expressed as a prepro-peptide that undergoes N-terminal and C-terminal proteolytic processing, and forms non-covalently linked dimers. VEGF-D stimulates mitogenic responses in endothelial cells in vitro. Exemplary human prepro-VEGF-D nucleic acid and amino acid sequences are set forth in SEQ ID NO:3 and SEQ ID NO:4, respectively. In addition, VEGF-D is described in greater detail in International Patent Publication No. WO 98/07832 and U.S. Pat. No. 6,235,713, each of which is incorporated herein by reference and describes VEGF-D polypeptides and variants thereof that are useful in producing the chimeras of the present invention. VEGF-D related molecules also are described in International Patent Publication Nos. WO 98/02543 and WO 97/12972, and U.S. Pat. No. 6,689,580, and U.S. patent application Ser. Nos. 09/219,345 and 09/847,524, all of which are incorporated by reference.

Isolation of a biologically active fragment of VEGF-D designated VEGF-DΔNΔC, is described in International Patent Publication No. WO 98/07832, incorporated herein by reference. VEGF-DΔNΔC consists of amino acid residues 93 to 201 of VEGF-D linked to the affinity tag peptide FLAG®. The prepro-VEGF-D polypeptide has a putative signal peptide of 21 amino acids and is apparently proteolytically processed in a manner analogous to the processing of prepro-VEGF-C. A “recombinantly matured” VEGF-D lacking residues 1-92 and 202-354 of SEQ ID NO: 4 retains the ability to activate receptors VEGFR-2 and VEGFR-3, and appears to associate as non-covalently linked dimers. Thus, preferred VEGF-D polynucleotides include those polynucleotides that comprise a nucleotide sequence encoding amino acids 93-201 of SEQ ID NO: 4, or comprising fragments thereof that retain VEGFR-3 and/or VEGFR-2 binding.

Moreover, since the therapeutic VEGF-D is to be administered as recombinant VEGF-D or indirectly via somatic gene therapy, it is within the skill in the art (and an aspect of the invention) to make and use analogs of human VEGF-D (and polynucleotides that encode such analogs) wherein one or more amino acids have been added, deleted, or replaced with other amino acids, especially with conservative replacements, and wherein the VEGFR-3 binding activity has been retained. Analogs that retain VEGFR-3 binding biological activity are contemplated as VEGF-D polypeptides for use in the present invention. In a preferred embodiment, analogs having 1, 2, 3, 4, 5, 6, 7, 8, 9, 10, 11, 12, 13, 14, 15, 16, 17, 18, 19, 20, 21, 22, 23, 24, or 25 such modifications and that retain VEGFR-3 binding activity are contemplated as VEGF-D polypeptides for use in the present invention. Polynucleotides encoding such analogs are generated using conventional PCR, site-directed mutagenesis, and chemical synthesis techniques. Molecules that bind and stimulate phosphorylation of VEGFR-3 are preferred.

Preferred fragments of VEGF-C or -D for use in making the chimeric molecules of the invention are continuous fragments that bind VEGFR-3. However, it has been demonstrated that VEGFR-3 binding can be achieved with molecules that incorporate discrete, discontinuous fragments of VEGF-C, fused, e.g., to fragments of VEGF-A or other amino acid sequences. Such chimeric VEGFR-3 ligands are described in U.S. patent application Ser. No. 09/795,006, filed Feb. 26, 2001, and International Patent Publication No. WO 01/62942, each of which is incorporated herein by reference in its entirety. The methods and compositions described in these documents may be used in the present invention to produce VEGF-C chimeras having a heparin binding domain. Moreover, the same teachings also apply to using continuous or discontinuous fragments of VEGF-D to make molecules that bind VEGFR-3.

In still another variation, the VEGFR-3 ligand sequence for use in making chimeras of the invention is itself a chimeric molecule comprised of VEGF-C and VEGF-D sequences. The foregoing documents describe methods for making such chimeras and confirming their VEGFR-3 binding activity.

In addition to binding VEGFR-3, the VEGFR-3 binding domain used to make molecules of the invention optionally also binds VEGFR-2. In addition, the molecule optionally binds VEGFR-1 and/or one or more neuropilin molecules.

Receptor binding assays for determining the binding of such chimeric molecules to one or more of these receptors are well-known in the art. Examples of such receptor binding assays are taught in e.g., U.S. patent application Ser. No. 09/795,006, and WO 01/62942, each incorporated herein by reference. (See, e.g., Example 3 of U.S. patent application Ser. No. 09/795,006, and WO 01/62942, which details binding assays of VEGF-C and related VEGF receptor ligands to soluble VEGF receptor-Fc fusion proteins. Example 5 of those documents details analysis of receptor activation or inhibition by such ligands. Example 6 describes analyses of receptor binding affinities of such ligands.) In addition, Achen et al., Proc Natl Acad Sci USA 95:548-53 (1998), incorporated by reference in its entirety, teaches exemplary binding assays. The binding of the chimeric VEGF polypeptides having the formula X-B-Z to any one or more of VEGF receptors, VEGFR-1, VEGFR-2, and VEGFR-3, may be analyzed using such exemplary assays.

Domain Z: a Heparin Binding Domain

Domain Z of the chimeric X-B-Z molecules is any substance that possesses heparin binding activity and therefore confers such heparin binding activity to the chimeric polypeptide. Without being bound to any mechanisms of action, it is contemplated that the presence of a heparin binding domain on the growth factors facilitates the binding of the growth factors to heparin and allows the concentration of the growth factors in the extracellular matrix to increase the efficiency of binding of the growth factors to their respective cell surface receptors, thereby increasing the bioavailability of the growth factors at a given site.

VEGF-C and VEGF-D, like VEGF₁₂₁, lack a heparin binding domain. However, it is known that VEGF₁₄₅, VEGF₁₆₅, VEGF₁₈₉ and VEGF₂₀₆, comprise heparin-binding domains (Keck et al., Arch. Bioch. Biophys., 344:103-113, 1997; Fairbrother et al., Structure 6:637-648, 1998). Exons 6 (21 amino acids) and 7 (44 amino acids) contain two independent heparin binding domains (Poltorak et al., Herz, 25:126-9, 2000). In preferred aspects of the present invention, subunit Z is a heparin binding domain encoded by exon 6, and/or exon 7 of VEGF. Subunit Z may further comprise the amino acids encoded by exon 8 of VEGF. The sequences of the various exons of VEGF are widely known and may be found at e.g., Genbank Accession numbers M63976-M63978, where M63976 is exon 6 (SEQ ID NO: 9), M63977 is exon 7 (SEQ ID NO: 11); and M63978 is exon 8 (SEQ ID NO: 13).

As noted herein, the human VEGF-A gene is expressed as numerous isoforms, including VEGF₁₄₅, VEGF₁₆₅, VEGF₁₈₉, and VEGF₂₀₆. A human VEGF₂₀₆ sequence obtained from the Swiss Prot database (accession no. P15692) is set forth below and in SEQ ID NO: 5:

| | |
|---|---|
| 1 | mnfllswvhw slalllylhh akwsqaapma egggqnhhev vkfmdvyqrs ychpietlvd |
| 61 | ifqeypdeie yifkpscvpl mrcggccnde glecvptees nitmqimrik phqgqhigem |
| 121 | sflqhnkcec rpkkdrarqe kksvrgkgkg qkrkrkksry kswsvyvgar cclmpwslpg |
| 181 | phpcgpcser rkhlfvqdpq tckcsckntd srckarqlel nertcrcdkp rr |

Amino acids 1-26 of this sequence represent the signal peptide and mature VEGF₂₀₆ comprises amino acids 27-232. Referring to the same sequence, the signal peptide and amino acids 142-226 are absent in mature isoform VEGF₁₂₁ (SEQ ID NO: 12). The signal peptide and amino acids 166-226 are absent in mature isoform VEGF₁₄₅ (SEQ ID NO: 14). The signal peptide and amino acids 142-182 are absent in mature isoform VEGF₁₆₅ (SEQ ID NOs: 6-7). The signal peptide and amino acids 160-182 are absent in mature isoform VEGF₁₈₃. The signal peptide and amino acids 166-182 are absent in mature isofrom VEGF₁₈₉.

Referring to FIG. 1B and the foregoing sequence, amino acids 142-165 correspond to exon 6a (found in VEGF isoforms 145, 189, and 206); amino acids 166-182 correspond to exon 6b (found in isofrom 206 only); and amino acids 183-226 correspond to exon 7 (found in isoforms 165, 189, and 206).

Thus, referring again to the same sequence, the apparent heparin binding domain within VEGF₁₄₅ corresponds to amino acids 142-165 or a fragment thereof. The apparent heparin binding domain of VEGF₁₆₅ corresponds to amino acids 183-226 or a fragment thereof.

The apparent heparin binding domain(s) of VEGF₁₈₉ (SEQ ID NO: 10) correspond to amino acids 142-165 joined directly to amino acids 183-226, or fragment(s)s thereof. The apparent heparin binding domain(s) of VEGF₂₀₆ correspond to amino acids 142-226, or fragment(s) thereof.

In other embodiments, subunit Z may be derived from the heparin binding domains of other, non-VEGF growth factors. For example, subunit Z may be the heparin binding domain of VEGF-B. Makinen et al., (J. Biol. Chem., 274:21217-22, 1999), have described various isoforms of VEGF-B and have shown that the exon 6B encoded sequence of VEGF-B₁₆₇ resembles the heparin and NRP1-binding domain encoded by exon 7 of VEGF₁₆₅. Thus exon-6B of VEGF-B₁₆₇ (or a heparin binding fragment thereof) may be used as the heparin binding subunit Z of the chimeric molecules of the present invention. The publication of Makinen et al., J. Biol. Chem., 274: 21217-22, 1999 provides a detailed description of the construction of the VEGF-B exon 6B-encoded sequence. Nucleotide and deduced amino acid sequences for VEGF-B are deposited in GenBank under Acc. No. U48801, incorporated herein by reference. Also incorporated herein by reference is Olofsson et al., J. Biol. Chem. 271 (32), 19310-19317 (1996), which describes the genomic organization of the mouse and human genes for VEGF-B, and its related Genbank entry at AF468110, which provides an exemplary genomic sequence of VEGF-B.

Mulloy et al., (Curr Opin Struct Biol. 11(5):623-8, 2001) describes properties from many heparin binding domain structures and identifies many heparin binding domain examples, and is incorporated herein by reference. Any such heparin binding domains may be used in the chimeric molecules of the present invention. In still further embodiments, subunit Z may comprise the heparin binding domain of PlGF-2 (see Hauser and Weich, Growth Factors, 9 259-68, 1993). Heparin binding domains from other growth factors also may be used in the present chimeric polypeptides, such as for example the heparin binding domain from EGF-like growth factor (Shin et al., J Pept Sci. 9(4):244-50, 2003); the heparin binding domain from insulin-like growth factor-binding protein (Shand et al., J Biol Chem. 278(20):17859-66, 2003), and the like. Other heparin binding domains that may be used herein include, but are not limited to, the pleiotrophin and amphoterin heparin binding domains (Matrix Biol. 19(5):377-87, 2000); CAP37 (Heinzelmann et al., Int J Surg Investig. 2(6):457-66, 2001); and the heparin-binding fragment of fibronectin (Yasuda et al., Arthritis Rheum. 48(5):1271-80, 2003).

Those of skill in the art are aware that heparin binding domains are present on numerous other proteins, including e.g., apolipoprotein E (SEQ ID NO: 61, residues 162-165, 229-236), fibronectin (SEQ ID NO: 62), amphoterin (SEQ ID NO: 63), follistatin (SEQ ID NO: 64), LPL (SEQ ID NO: 65), myeloperoxidase (SEQ ID NO: 66), other growth factors, and the like. Merely by way of example, the protein sequences of various heparin binding proteins found in Genbank include but are not limited to 1LR7_A; 1LR8_A; 1LR9_A; AAH05858 (FN1, SEQ ID NO: 58); NP_{—}000032 (SEQ ID NO: 54); NP_{—}000177 (H Factor 1, SEQ ID NO: 52); NP_{—}001936 (dip theria toxin receptor, SEQ ID NO: 51); NP_{—}002328 (alpha-2-MRAP, SEQ ID NO: 53); NP_{—}005798 (proteoglycan 4, SEQ ID NO: 55); NP_{—}009014 (SEQ ID NO: 36); NP_{—}032018; NP_{—}032511; NP_{—}034545; NP_{—}035047; NP_{—}037077; NP_{—}498403; NP_{—}604447; NP_{—}932158 (SEQ ID NO: 37); NP_{—}990180; O15520 (SEQ ID NO: 50); O35565; O46647; P01008 (SEQ ID NO: 40); P02649 (SEQ ID NO: 35); P02749 (SEQ ID NO: 39); P02751 (SEQ ID NO: 59); P04196 (SEQ ID NO: 42); P04937; P05546 (SEQ ID NO: 56); P05770; P06858 (SEQ ID NO: 44); P07155; P07589; P08226; P10517; P11150(SEQ ID NO: 43); P11276; P11722_{—}1; P11722_{—}2; P15656; P15692 (SEQ ID NO: 57); P17690; P18287; P18649; P18650; P20160 (SEQ ID NO: 38); P23529; P26644; P27656; P30533 (SEQ ID NO: 45); P33703; P35268 (SEQ ID NO: 49); P47776; P49182; P49763 (SEQ ID NO: 47); P51858 (SEQ ID NO: 41); P51859; P55031; P61150; P61328; P61329; Q01339; Q01580; Q06186; Q11142; Q15303; Q28275; Q28377; Q28502; Q28640; Q28995; Q61092; Q61851; Q64268; Q7M2U7; Q8VHK7; Q91740; Q95LB0; Q99075 (SEQ ID NO: 46); Q9GJU3; Q9WVG5; Q9Y5X9 (SEQ ID NO: 48); XP_{—}357846; XP_{—}357859; XP_{—}358238; XP_{—}358249; 1304205A (SEQ ID NO: 31); 1AE5 (SEQ ID NO: 30); 1B9Q_A; 1FNH_A (SEQ ID NO: 29); 1KMX_A (SEQ ID NO: 28); 1MKC_A; 1OKQ_A; A35969 (SEQ ID NO: 21); A38432 (SEQ ID NO: 22); A41178 (SEQ ID NO: 23); A41914; A48991; AAA37542; AAA50562 (SEQ ID NO: 34); AAA50563 (SEQ ID NO: 32); AAA50564 (SEQ ID NO: 33); AAA81780; AAB27481; AAB33125; AAC42069; AAD29416; B40080; C40862 (SEQ ID NO: 60); I39383 (SEQ ID NO: 24); IB9P_A; JC1409; JC1410; JC4168; JT0573; LPHUB (SEQ ID NO: 25); LPHUE (SEQ ID NO: 26); O18739; O19113; P11151; P11153; P11602; P12034 (SEQ ID NO: 27); P13387; P41104; P48807; P49060; P49923; P55302; P70492; Q06000; Q06175; Q09118; Q11184; Q29524; Q91289; Q9CB42; Q9R1E9; S26049; S27162; S51242; XP_{—}134550; XP_{—}142078; XP_{—}145641; XP_{—}212881; XP_{—}213021; XP_{—}227645; XP_{—}232701; XP_{—}344685; XP_{—}344947; XP_{—}345821; XP_{—}346046; XP_{—}357159; XP_{—}357228; XP_{—}357258; XP_{—}358223. In addition, the heparin binding domain may be one derived from any of these proteins. In exemplary embodiments heparin binding of the domain may be determined by e.g., heparin affinity chromatography. In alternative embodiments, the heparin binding domain may be assessed using methods described in U.S. Pat. No. 6,274,704. The heparin binding peptides described therein also may by useful.

Domain B: a Covalent Linkage between X and Z.

Within the chimeric molecules of the formula X-B-Z, the term B denotes a linkage, preferably a covalent linkage, between subunit X and subunit Z. In some embodiments, B simply denotes a covalent bond. For example, in a preferred embodiment, where X-B-Z comprises a single continuous polypeptide, B can denote an amide bond between the C-terminal amino acid of X and the N-terminal.amino acid of Z, or between the C-terminal amino acid of Z and the N-terminal amino acid of X. Another way to describe such embodiments is by the simplified formulas X-Z or Z-X.

The linker may be an organic moiety constructed to contain an alkyl, aryl backbone and may contain an amide, ether, ester, hydrazone, disulphide linkage or any combination thereof. Linkages containing amino acid, ether and amide bound components will be stable under conditions of physiological pH, normally 7.4 in serum and 4-5 on uptake into cells (endosomes). Preferred linkages are linkages containing esters or hydrazones that are stable at serum pH but hydrolyse to release the drug when exposed to intracellular pH. Disulphide linkages are preferred because they are sensitive to reductive cleavage; amino acid linkers can be designed to be sensitive to cleavage by specific enzymes in the desired target organ. Exemplary linkers are set out in Blattler et al. Biochem. 24:1517-1524, 1985; King et al. Biochem. 25:5774-5779, 1986; Srinivasachar and Nevill, Biochem. 28:2501-2509, 1989.

In still other embodiments, entity B is a chemically, or otherwise, cleavable bond that, under appropriate conditions, allows the release of subunit X from subunit Z. For example domains X and Z can be covalently linked by one or more disulfide bridges linking cysteine residues of X and Z; or by mutual attachment to a distinct chemical entity, such as a carbohydrate moiety.

In particular embodiments, entity B comprises a peptide linker comprising from 1 to about 500 amino acids in length. Linkers of 4-50 amino acids are preferred, and 4-15 are highly preferred. Preferred linkers are joined N-terminally and C-terminally to domains X and Z so as to form a single continuous polypeptide. In certain embodiments, the peptide linker comprises a protease cleavage site selected from the group consisting of a Factor Xa cleavage site, an enterokinase cleavage site (New England Biolabs), a thrombin cleavage site, a TEV protease cleavage site (Life Technologies), and a PreScission cleavage site (Amersham Pharmacia Biotech). The presence of such cleavage sites between subunit X and subunit Z will allow for the efficient release of effective amounts of subunit X in a suitable proteolytic milieu.

Processing of VEGF-C and -D is believed to occur in part intracellularly, but processing of the amino terminal pro-peptide is believed to occur following secretion. Cleavage of this pro-peptide is apparently necessary for VEGFR-2-mediated activity. In one variation of the invention, subunit B comprises an amino acid sequence analogous to the VEGF-C or -D N-terminal pro-peptide processing site, to make subunits X and Z susceptible to cleavage by the same protease that process these N-terminal pro-peptide in vivo.

For example, with respect to VEGF-C, propeptide cleavage can occur at about amino acids 102/103 of SEQ ID NO: 2, and a suitable subunit B optionally include about 3-30 amino acids upstream and downstream of this site. The analogous processing site of VEGF-D occurs between residues 92 and 93 of SEQ ID NO: 4.

The linker is optionally a heterologous protein polypeptide. The linker may affect whether the polypeptide(s) to which it is fused to is able to dimerize to each other or to another polypeptide. Other chemical linkers are possible, as the linker need not be in the form of a polypeptide. However, when the linker comprises a peptide, the binding construct (with linker) allows for expression as a single molecule. Linker may be chosen such that they are less likely to induce an allergic or antigenic reaction.

More than one linker may be used per molecule of X-B-Z or Z-B-X. The linker may be selected for optimal conformational (steric) freedom between the growth factor and heparin binding domains allow them to interact with binding partners. The linker may be linear such that X and Z are linked in series, or the linker may serve as a scaffold to which two or more X or Z binding units are attached. A linker may also have multiple branches. For example, using linkers disclosed in Tam, J. Immunol. Methods 196:17 (1996). X or Z domains may be attached to each other or to the linker scaffold via N-terminal amino groups, C-terminal carboxyl groups, side chains, chemically modified groups, side chains, or other means.

When comprising peptides, the linker may be designed to have sequences that permit desired characteristics. For example, the use of glycyl residues allow for a relatively large degree of conformational freedom, whereas a proline would tend to have the opposite effect. Peptide linkers may be chosen so that they achieve particular secondary and tertiary structures, e.g., alpha helices, beta sheets and beta barrels. Quarternary structure can also be utilized to create linkers that join two binding units together non-covalently. For example, fusing a protein domain with a hydrophobic face to each binding unit may permit the joining of the two binding units via the interaction between the hydrophobic interaction of the two molecules. In some embodiments, the linker may provide for polar interactions. For example, a leucine zipper domain of the proto-oncoproteins Myc and Max, respectively may be used. Luscher and Larsson, Ongogene 18:2955-2966 (1999). In some embodiments, the linker allows for the formation of a salt bridge or disulfide bond. Linkers may comprise non-naturally occurring amino acids, as well as naturally occurring amino acids that are not naturally incorporated into a polypeptide. In some embodiments, the linker comprises a coordination complex between a metal or other ions and various residues from the multiple peptides joined thereby.

Linear peptide linkers may have various lengths, and generally consist of at least one amino acid residue. In some embodiments the linker has from 1 to 10 residues. In some embodiments, the linker has from 1 to 50 residues. In some embodiments, the linker has from 1 -100 residues. In some embodiments, the linker has from 1-1000 residues. In some embodiments the linker has 1-10,000 residues. In some embodiments the linker has more than 10,000 residues. In some embodiments, the linear peptide linker comprises residues with relatively inert side chains. Peptide linker amino acid residues need not be linked entirely or at all via alpha-carboxy and alpha-amino groups. That is, peptides may be linked via side chain groups of various residues. In some embodiments, a linker is used as is described in Liu et al. U.S. Pat. Appl. Pub. No. 2003/0064053.

B. Methods of Making Chimeric VEGF Polypeptides

The chimeric molecules of the invention can be synthesized in solution or on a solid support in accordance with conventional techniques. Such polypeptides may be synthesized as small fragments of the complete chimeric polypeptide or as a complete full length sequence. Various automatic synthesizers are commercially available and can be used in accordance with known protocols. See, for example, Stewart and Young, Solid Phase Peptide Synthesis, 2d. ed., Pierce Chemical Co., (1984);Tam et al., J. Am. Chem. Soc., 105:6442, (1983); Merrifield, Science, 232: 341-347, (1986); and Barany and Merrifield, The Peptides, Gross and Meienhofer, eds, Academic Press, New York, 1-284, (1979), each incorporated herein by reference. The chimeric VEGF polypeptides of the invention having the formula X-B-Z or Z-B-X, can be readily synthesized and then screened using any of a number of assays that identify the polypeptides for VEGF-C-like, VEGF-D-like or other VEGF-like activity, such as e.g., binding to VEGFR-1, VEGFR-2, or VEGFR-3, induction of vascular permeability, activity in an endothelial cell proliferation assay, induction of growth of lymphatic vessels, promotion of growth and differentiation of CD34+ progenitor cells in vitro, activity in CAM assays, and the like. These and other assays for determining the activity of the vascular endothelial growth factor activity are described in U.S. patent application Ser. No. 09/795,006, and WO 01/62942.

Examples of solid-phase technology that may be used in the present invention include a Model 433A from Applied Biosystems Inc peptide synthesizer. Methods of using such automated solid phase synthesizers to produce pure polypeptides are well known.

As an alternative to automated peptide synthesis, recombinant DNA technology may be employed wherein a nucleotide sequence which encodes a chimeric polypeptide of the invention is inserted into an expression vector, transformed or transfected into an appropriate host cell and cultivated under conditions suitable for expression. Recombinant methods are especially preferred for producing longer polypeptides that comprise peptide sequences of the invention. Chimeric molecules of the invention also may be produced by a combination of techniques whereby domains are synthesized recombinantly or synthetically in two or more steps and joined together as a single polypeptide.

A variety of expression vector/host systems may be utilized to contain and express the chimeric polypeptide coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the polypeptides in bacteria, yeast and other invertebrates are described herein below.

Expression vectors for use in prokaryotic hosts generally comprise one or more phenotypic selectable marker genes. Such genes generally encode, e.g., a protein that confers antibiotic resistance or that supplies an auxotrophic requirement. A wide variety of such vectors are readily available from commercial sources. Examples include pSPORT vectors, pGEM vectors (Promega), pPROEX vectors (LTI, Bethesda, Md.), Bluescript vectors (Stratagene), pET vectors (Novagen) and pQE vectors (Qiagen). The DNA sequence encoding a peptide domain or chimeric polypeptide is cloned into such a vector, for example, pGEX-3X (Pharmacia, Piscataway, N.J.) designed to produce a fusion protein comprising glutathione-S-transferase (GST), encoded by the vector, and a protein encoded by a DNA fragment inserted into the vector's cloning site. Treatment of the recombinant fusion protein with thrombin or factor Xa (Pharmacia, Piscataway, N.J.) is expected to cleave the fusion protein, releasing the polypeptide of interest from the GST portion. The pGEX-3X/chimeric VEGF polypeptide construct is transformed into E. coli XL-1 Blue cells (Stratagene, La Jolla Calif.), and individual transformants were isolated and grown. Plasmid DNA from individual transformants is purified and partially sequenced using an automated sequencer to confirm the presence of the desired peptide or polypeptide encoding nucleic acid insert in the proper orientation.

Induction of the GST/substrate fusion protein is achieved by growing the transformed XL-1 Blue culture at 37° C. in LB medium (supplemented with carbenicillin) to an optical density at wavelength 600 nm of 0.4, followed by further incubation for 4 hours in the presence of 0.5 mM Isopropyl β-D-Thiogalactopyranoside (Sigma Chemical Co., St. Louis Mo.).

The GST fusion protein, expected to be produced as an insoluble inclusion body in the bacteria, may be purified as follows. Cells are harvested by centrifugation; washed in 0.15 M NaCl, 10 mM Tris, pH 8, 1 mM EDTA; and treated with 0.1 mg/ml lysozyme (Sigma Chemical Co.) for 15 minutes at room temperature. The lysate is cleared by sonication, and cell debris is pelleted by centrifugation for 10 minutes at 12,000×g. The fusion protein-containing pellet is resuspended in 50 mM Tris, pH 8, and 10 mM EDTA, layered over 50% glycerol, and centrifuged for 30 min. at 6000×g. The pellet is resuspended in standard phosphate buffered saline solution (PBS) free of Mg⁺⁺ and Ca⁺⁺. The fusion protein is further purified by fractionating the resuspended pellet in a denaturing SDS polyacrylamide gel (Sambrook et al., supra). The gel is soaked in 0.4 M KCl to visualize the protein, which is excised and electroeluted in gel-running buffer lacking SDS. If the GST/chimeric VEGF polypeptide fusion protein is produced in bacteria as a soluble protein, it may be purified using the GST Purification Module (Pharmacia Biotech).

The fusion protein may be subjected to thrombin digestion to cleave the GST from the chimeric VEGF polypeptide. The digestion reaction (20-40 μg fusion protein, 20-30 units human thrombin (4000 U/mg (Sigma) in 0.5 ml PBS) is incubated 16-48 hrs. at room temperature and loaded on a denaturing SDS-PAGE gel to fractionate the reaction products. The gel is soaked in 0.4 M KCl to visualize the protein bands. The identity of the protein band corresponding to the expected molecular weight of the chimeric VEGF polypeptide may be confirmed by partial amino acid sequence analysis using an automated sequencer (Applied Biosystems Model 473A, Foster City, Calif.).

Alternatively, the DNA sequence encoding the predicted substrate containing fusion polypeptide may be cloned into a plasmid containing a desired promoter and, optionally, a leader sequence (see, e.g., Better et al., Science, 240: 1041 43, 1988). The sequence of this construct may be confirmed by automated sequencing. The plasmid is then transformed into E. coli using standard procedures employing CaCl₂ incubation and heat shock treatment of the bacteria (Sambrook et al., supra). The transformed bacteria are grown in LB medium supplemented with carbenicillin, and production of the expressed protein is induced by growth in a suitable medium. If present, the leader sequence will effect secretion of the chimeric VEGF polypeptide and be cleaved during secretion. The secreted recombinant protein may then be purified using conventional protein purification techniques.

Similarly, yeast host cells from genera including Saccharomyces, Pichia, and Kluveromyces may be employed to generate the peptide recombinantly. Preferred yeast hosts are S. cerevisiae and P. pastoris. Yeast vectors will often contain an origin of replication sequence from a 2T yeast plasmid, an autonomously replicating sequence (ARS), a promoter region, sequences for polyadenylation, sequences for transcription termination, and a selectable marker gene. Vectors replicable in both yeast and E. coli (termed shuttle vectors) may also be used. In addition to the above-mentioned features of yeast vectors, a shuttle vector will also include sequences for replication and selection in E. coli. Direct secretion of polypeptides expressed in yeast hosts may be accomplished by the inclusion of nucleotide sequence encoding the yeast I-factor leader sequence at the 5′ end of the substrate-encoding nucleotide sequence.

Generally, a polypeptide is recombinantly expressed in yeast using a commercially available expression system, e.g., the Pichia Expression System (Invitrogen, San Diego, Calif.), following the manufacturer's instructions. This system also relies on the pre pro alpha sequence to direct secretion, but transcription of the insert is driven by the alcohol oxidase (AOX1) promoter upon induction by methanol.

The secreted recombinant substrate is purified from the yeast growth medium by, e.g., the methods used to purify substrate from bacterial and mammalian cell supernatants.

Alternatively, the chimeric VEGF polypeptide may be expressed in an insect system. Insect systems for protein expression are well known. In one such system, Autographa californica nuclear polyhedrosis virus (AcNPV) is used as a vector to express foreign genes in Spodoptera frugiperda cells or in Trichoplusia larvae. The polypeptide coding sequence is cloned into a nonessential region of the virus, such as the polyhedrin gene, and placed under control of the polyhedrin promoter. Successful insertion of substrate will render the polyhedrin gene inactive and produce recombinant virus lacking protein coat. The recombinant viruses are then used to infect S. frugiperda cells or Trichoplusia larvae in which the substrate is expressed (Smith et al., J Virol 46: 584, 1983; Engelhard E K et al., Proc Nat Acad Sci 91: 3224-7, 1994). For example, DNA encoding a polypeptide of the invention may be cloned into the baculovirus expression vector pVL1393 (PharMingen, San Diego, Calif.; Luckow and Summers, Bio/Technology 6:47 (1988)). This resulting vector is then used according to the manufacturer's directions (PharMingen) to infect Spodoptera frugiperda cells in SF9 protein free media and to produce recombinant protein. The protein or peptide is purified and concentrated from the media using a heparin Sepharose column (Pharmacia, Piscataway, N.J.) and sequential molecular sizing columns (Amicon, Beverly, Mass.), and resuspended in PBS. SDS PAGE analysis shows a single band and confirms the size of the protein, and Edman sequencing on a Porton 2090 Peptide Sequencer confirms its N terminal sequence.

Mammalian host systems for the expression of recombinant proteins also are well known. Host cell strains may be chosen for a particular ability to process the expressed protein or produce certain post translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, W138, and the like have specific cellular machinery and characteristic mechanisms for such post-translational activities and may be chosen to ensure the correct modification and processing of the introduced, foreign protein.

It is preferable that the transformed cells are used for long-term, high-yield protein production and as such stable expression is desirable. Once such cells are transformed with vectors that contain selectable markers along with the desired expression cassette, the cells may be allowed to grow for 1-2 days in an enriched media before they are switched to selective media. The selectable marker is designed to confer resistance to selection and its presence allows growth and recovery of cells which successfully express the introduced sequences. Resistant clumps of stably transformed cells can be proliferated using tissue culture techniques appropriate to the cell.

A number of selection systems may be used to recover the cells that have been transformed for recombinant protein production. Such selection systems include, but are not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to methotrexate; gpt, that confers resistance to mycophenolic acid; neo, that confers resistance to the aminoglycoside G418; als which confers resistance to chlorsulfuron; and hygro, that confers resistance to hygromycin. Additional selectable genes that may be used include trpB, which allows cells to utilize indole in place of tryptophan, or hisD, which allows cells to utilize histinol in place of histidine. Markers that give a visual indication for identification of transformants include anthocyanins, b-glucuronidase and its substrate, GUS, and luciferase and its substrate, luciferin.

Protein Purification.

It will be desirable to purify the chimeric VEGF polypeptide of the present invention. Protein purification techniques are well known. These techniques involve, at one level, the crude fractionation of the cellular milieu to polypeptide and non-polypeptide fractions. Having separated the peptide or polypeptides of the invention from other proteins, the polypeptides or peptides of interest may be further purified using chromatographic and electrophoretic techniques to achieve partial or complete purification (or purification to homogeneity).

Generally, “purified” will refer to a polypeptide, protein or peptide composition that has been subjected to fractionation to remove various other components, and which composition substantially retains its expressed biological activity. Where the term “substantially purified” is used, this designation will refer to a composition in which the polypeptide, protein or peptide forms the major component of the composition, such as constituting about 50%, about 60%, about 70%, about 80%, about 90%, about 95% or more of the proteins in the composition.

Various methods for quantifying the degree of purification of the polypeptide, protein or peptide will be apparent. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed polypeptide, protein or peptide exhibits a detectable activity.

Various techniques known for use in protein purification are also suitable for molecules of the present invention. These include, for example, precipitation with ammonium sulphate, PEG, antibodies and the like or by heat denaturation, followed by centrifugation; chromatography steps such as ion exchange, gel filtration, reverse phase, hydroxylapatite, exclusion, and affinity chromatography; isoelectric focusing; gel electrophoresis (including polyacrylamide gel electrophoresis); and combinations of such and other techniques. The order of conducting the various purification steps may be varied, and certain steps may be omitted, and still result in a suitable method for the preparation of a substantially purified polypeptide, protein or peptide.

There is no general requirement that the polypeptide, protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., Biochem. Biophys. Res. Comm., 76:425, 1977). It will therefore be appreciated that under differing electrophoresis conditions, the apparent molecular weights of purified or partially purified expression products may vary.

In still another related embodiment, the invention provides a method for producing a vascular endothelial growth factor receptor binding protein, comprising the steps of growing a host cell of the invention in a nutrient medium and isolating the polypeptide or variant thereof from the cell or the medium. Isolation of the polypeptide from the cells or from the medium in which the cells are grown is accomplished by purification methods known in the art, e.g., conventional chromatographic methods including immunoaffinity chromatography, receptor affinity chromatography, hydrophobic interaction chromatography, lectin affinity chromatography, size exclusion filtration, cation or anion exchange chromatography, high pressure liquid chromatography (HPLC), reverse phase HPLC, and the like. Still other methods of purification include those wherein the desired protein is expressed and purified as a fusion protein having a specific tag, label, or chelating moiety that is recognized by a specific binding partner or agent. The purified protein can be cleaved to yield the desired protein, or be left as an intact fusion protein. Cleavage of the fusion component may produce a form of the desired protein having additional amino acid residues as a result of the cleavage process.

In preferred embodiments, purification of the chimeric polypeptides of the present invention may be achieved using affinity purification using an extracellular domain of Flt4, or other portion of a receptor that the chimeric polypeptides of the invention may bind. Exemplary affinity purification of VEGF-C related compositions is described in e.g., U.S. Pat. No. 5,776,755, incorporated herein by reference. In an exemplary affinity purification procedure using the FLT4 extracellular domain, the chimeric polypeptide-containing composition to be purified are initially concentrated 30-50 fold using Centriprep filter cartridges and loaded onto a column of immobilized FLT4 extracellular domain. Two affinity matrices are prepared. In the first case, the Flt4EC-6×His fusion protein is crosslinked to CNBr-activated Sepharose 4B (Pharmacia) and in the second case the FLT4-Ig fusion protein is coupled to protein A Sepharose using dimethylpimelidate (Schneider et al, J. Biol. Chem. 257: 10766-10769, 1982). The material eluted from the affinity column is subjected to further purification using ion exchange and reverse-phase high pressure chromatography and SDS-polyacrylamide gel electrophoresis.

As the chimeric polypeptides of the present invention have the ability to bind VEGFR-3 and have the ability to bind heparin, one method of obtaining a highly purified specimen would be to subject the chimeric polypeptides to two types of affinity purification. One affinity purification being based on VEGFR-3 binding property of the chimeric polypeptides and the second affinity purification being based on the heparin binding property of the chimeric polypeptides. Heparin-based affinity chromatography methods are well known. For example, one uses a commercially available heparin-Sepharose affinity chromatography system such as e.g., Heparin Sepharose™ 6 Fast Flow available from Amersham Biosciences (Piscataway, N.J.). Heparin Sepharose also is available from Pharmacia (Uppsula, Sweden). Other heparin affinity chromatography resins are available from Sigma Aldrich (St. Louis, Mo.). Exemplary protocols for purifying VEGF165 using Heparin-Sepharose CL6B affinity chromatography are presented by Ma et al., (Biomed Environ Sci. 14(4):302-11, 2001), Dougher et al., (Growth Factors, 14(4):257-68, 1997). Such methods could be used for the purification of the chimeric polypeptides of the present invention. Where these methods are used in conjunction with the FLT4 receptor-based affinity purification discussed above, the receptor-based affinity purification may be performed before or after the heparin binding affinity chromatography step.

Yet another affinity chromatography purification procedure that may be used to purify the chimeric polypeptides of the present invention employs immunoaffinity chromatography using antibodies specific for either the heparin binding domain of the chimeric polypeptides or more preferably antibodies specific for the domain X of the chimeric polypeptides. Antibodies specific for domain X would be any antibodies that are specific for VEGF-C, VEGF-D or chimeras of VEGF-D. In addition, purification of the chimeric polypeptides of the present invention may be achieved using methods for the purification of VEGF-C or VEGF-D that are described in U.S. Pat. No. 6,361,946 and WO 98/07832, respectively.

C. Nucleic Acids and Related Compositions.

The invention embraces polynucleotides that encode the chimeric VEGF polypeptides discussed above and also polynucleotides that hybridize under moderately stringent or high stringency conditions to the complete non-coding strand, or complement, of such polynucleotides. Due to the well-known degeneracy of the universal genetic code, one can synthesize numerous polynucleotide sequences that encode each chimeric polypeptide of the present invention. All such polynucleotides are contemplated to be useful in the present application. Particularly preferred polynucleotides join a natural human VEGFR-3 receptor ligand cDNA sequence e.g., a sequence of SEQ ID NO:1 or SEQ ID NO:3, preferably a fragment thereof encoding a VEGFR-3 binding domain, with a natural human heparin binding domain encoding sequence. This genus of polynucleotides embraces polynucleotides that encode polypeptides with one or a few amino acid differences (additions, insertions, or deletions) relative to amino acid sequences specifically taught herein. Such changes are easily introduced by performing site directed mutagenesis, for example.

One genus of both polynucleotides of the invention and polypeptides encoded thereby can be defined by molecules with a first domain that hybridize under specified conditions to a VEGF-C or -D polynucleotide sequence and a second domain that hybridizes under the same conditions to naturally occurring human sequences that encode heparin binding domains taught herein.

Exemplary highly stringent hybridization conditions are as follows: hybridization at 65° C. for at least 12 hours in a hybridization solution comprising 5×SSPE, 5× Denhardt's, 0.5% SDS, and 2 mg sonicated non homologous DNA per 100 ml of hybridization solution; washing twice for 10 minutes at room temperature in a wash solution comprising 2×SSPE and 0.1% SDS; followed by washing once for 15 minutes at 65° C. with 2×SSPE and 0.1% SDS; followed by a final wash for 10 minutes at 65° C. with 0.1×SSPE and 0.1% SDS. Moderate stringency washes can be achieved by washing with 0.5×SSPE instead of 0.1×SSPE in the final 10 minute wash at 65° C. Low stringency washes can be achieved by using 1×SSPE for the 15 minute wash at 65° C., and omitting the final 10 minute wash. It is understood in the art that conditions of equivalent stringency can be achieved through variation of temperature and buffer, or salt concentration as described Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook et al., (Eds.), Molecular Cloning: A Laboratory Manual, Cold Spring Harbor Laboratory Press: Cold Spring Harbor, N.Y. (1989), pp. 9.47 to 9.51. For example, the invention provides a polynucleotide that comprises a nucleotide sequence that hybridizes under moderately stringent or high stringency hybridization conditions to any specific nucleotide sequence of the invention, and that encodes a chimeric polypeptide as described herein that binds at least one of the naturally occurring vascular endothelial growth factor or platelet derived growth factor receptors.

In a related embodiment, the invention provides a polynucleotide that comprises a nucleotide sequence that is at least 80%, 85%, 90%, 95%, 97%, 98%, or. 99% identical to any specific nucleotide sequence of the invention, and that encodes a polypeptide that binds heparin and at least one of the naturally occurring vascular endothelial growth factor or platelet derived growth factor receptors.

In a related embodiment, the invention provides vectors comprising a polynucleotide of the invention. Such vectors are useful, e.g., for amplifying the polynucleotides in host cells to create useful quantities thereof. In preferred embodiments, the vector is an expression vector wherein the polynucleotide of the invention is operatively linked to a polynucleotide comprising an expression control sequence. Autonomously replicating recombinant expression constructs such as plasmid and viral DNA vectors incorporating polynucleotides of the invention are specifically contemplated. Expression control DNA sequences include promoters, enhancers, and operators, and are generally selected based on the expression systems in which the expression construct is to be utilized. Preferred promoter and enhancer sequences are generally selected for the ability to increase gene expression, while operator sequences are generally selected for the ability to regulate gene expression. Expression vectors are useful for recombinant production of polypeptides of the invention. Expression constructs of the invention may also include sequences encoding one or more selectable markers that permit identification of host cells bearing the construct. Expression constructs may also include sequences that facilitate, and preferably promote, homologous recombination in a host cell. Preferred constructs of the invention also include sequences necessary for replication in a host cell.

Vectors also are useful for “gene therapy” treatment regimens, wherein a polynucleotide that encodes a polypeptide of the invention is introduced into a subject in need of treatment involving the modulation (stimulation or blockage) of vascular endothelial growth factor receptors, in a form that causes cells in the subject to express the polypeptide of the invention in vivo. Gene therapy aspects that are described in e.g., U.S. patent application Ser. No. 09/795,006, and WO 01/62942, also are applicable herein.

In another related embodiment, the invention provides host cells, including prokaryotic and eukaryotic cells, that are transformed or transfected (stably or transiently) with polynucleotides of the invention or vectors of the invention. Polynucleotides of the invention may be introduced into the host cell as part of a circular plasmid, or as linear DNA comprising an isolated protein coding region or a viral vector. Methods for introducing DNA into the host cell, which are well known and routinely practiced in the art include transformation, transfection, electroporation, nuclear injection, or fusion with carriers such as liposomes, micelles, ghost cells, and protoplasts. As stated above, such host cells are useful for amplifying the polynucleotides and also for expressing the polypeptides of the invention encoded by the polynucleotide. Such host cells are useful in assays as described herein. For expression of polypeptides of the invention, any host cell is acceptable, including but not limited to bacterial, yeast, plant, invertebrate (e.g., insect), vertebrate, and mammalian host cells. For developing therapeutic preparations, expression in mammalian cell lines, especially human cell lines, is preferred. Use of mammalian host cells is expected to provide for such post-translational modifications (e.g., glycosylation, truncation, lipidation, and phosphorylation) as may be desirable to confer optimal biological activity on recombinant expression products of the invention. Glycosylated and non-glycosylated forms of polypeptides are embraced by the present invention. Similarly, the invention further embraces polypeptides described above that have been covalently modified to include one or more water soluble polymer attachments such as polyethylene glycol, polyoxyethylene glycol, or polypropylene glycol.

Also within the scope of the invention are compositions comprising polypeptides or polynucleotides of the invention. In a preferred embodiment, such compositions comprise one or more polynucleotides or polypeptides of the invention that have been formulated with a pharmaceutically acceptable (e.g., sterile and non toxic) diluent or carrier. Liquid, semisolid, or solid diluents that serve as pharmaceutical vehicles, excipients, or media are preferred. Any diluent known in the art may be used. Exemplary diluents include, but are not limited to, water, saline solutions, polyoxyethylene sorbitan monolaurate, magnesium stearate, methyl and propylhydroxybenzoate, talc, alginates, starches, lactose, sucrose, dextrose, sorbitol, mannitol, glycerol, calcium phosphate, mineral oil, and cocoa butter. Such formulations are useful, e.g., for administration of polypeptides or polynucleotides of the invention to mammalian (including human) subjects in therapeutic regimens.

Similarly, the invention provides for the use of polypeptides or polynucleotides of the invention in the manufacture of a medicament for the treatment of disorders described herein, including but not limited to disorders characterized by insufficient or undesirable endothelial cell proliferation and/or disorders characterized by ischemia and/or vessel occlusion, wherein neovascularization is desirable.

In a related embodiment, the invention provides a kit comprising a polynucleotide, polypeptide, or composition of the invention packaged in a container, such as a vial or bottle, and further comprising a label attached to or packaged with the container, the label describing the contents of the container and providing indications and/or instructions regarding use of the contents of the container to treat one or more disease states as described herein.

In yet another aspect, the present invention provides methods of producing polypeptides having novel VEGF receptor binding and stimulation properties, and methods for producing polynucleotides that encodes such polypeptides.

As used herein, “modulate the growth of mammalian endothelial cells” means stimulate such growth by inducing a mitogenic signal through binding cell surface receptors expressed on vascular endothelial cells, or inhibiting such growth. The inhibition may be due to blockage of vascular or lymphatic endothelial growth factor receptors, or the formation of heterodimers with endogenous growth factors that prevent stimulation of endogenous receptors by the endogenous growth factors. Inhibition also may be achieved by conjugating cytotoxic agents to polypeptides of the invention that bind VEGF receptors. Exemplary toxins are known in the art and described elsewhere herein. Polypeptides of the invention conjugated to cytotoxic agents or other agents that modulate cell growth are contemplated as another aspect of the invention. Agonist molecules of the invention that stimulate endothelial cell growth are a preferred class of agents. Antagonists that inhibit endothelial cell growth also are preferred.

D. Methods of Using Chimeric VEGF-C and -D Polypeptides

In yet another embodiment, the invention provides numerous in vitro and in vivo methods of using the chimeric polypeptides and polynucleotides of the invention. Generally speaking, the chimeric polypeptides of the invention are useful for modulating (stimulating or inhibiting) cellular processes that are mediated through any of the PDGF/VEGF family of receptors, such as for example, VEGFR-1, more preferably, VEGFR-2, and/or VEGFR-3. These receptors may be involved singularly in certain processes and in combination, to varying extents, in other processes. The chimeric polypeptides of the invention advantageously have a heparin binding domain which increases the potency of the VEGFR ligand in the biological processes in which it is involved.

Thus, in one variation, the invention provides a method of modulating the signaling of one or more of receptors for which either VEGF-C or VEGF-D are ligands. The method generally involves the step of contacting a cell that expresses such a receptor, e.g., VEGFR-2, and/or VEGFR-3 with a composition comprising a chimeric polypeptide of the invention. In one variation, modulation to activate signaling is contemplated, and the cell is contacted with a polypeptide of the invention that stimulates receptor signaling in an amount sufficient to bind to the one or more receptors and induce receptor signaling. Preferably, an amount is employed that is effective to stimulate a cellular response such as an in vitro or in vivo endothelial cell proliferation and/or recruitment or angiogenesis or lymphangiogenesis.

In another variation, modulation to inhibit signaling is contemplated. The cell is contacted with a polypeptide that inhibits ligand-induced receptor activation (or a polypeptide conjugated to a cytotoxin), in an amount sufficient to inhibit signaling that is induced by receptor ligand growth factor polypeptides that exist endogenously in the cell's environment. Dose-response studies permit accurate determination of a proper quantity of chimeric polypeptide to employ. Effective quantities can be estimated from measurements of the binding affinity of a polypeptide for a target receptor, of the quantity of receptor present on target cells, of the expected dilution volume (e.g., patient weight and blood volume for in vivo embodiments), and of polypeptide clearance rates. Existing literature regarding dosing of known VEGFR ligands also provides guidance for dosing of molecules of the invention.

In another variation, the invention provides a method of modulating the signaling of one or more of the receptors of VEGF-C or VEGF-D in vivo, comprising the step or administering to a mammalian subject in need of modulation of the signaling of one or more of these receptors a composition comprising a polynucleotide of the invention, under conditions in which cells of the subject are transformed or transfected by the polynucleotide and express the chimeric polypeptide of the invention encoded thereby, wherein the expressed chimeric polypeptide modulates signaling of the one or more receptors. Human subjects are preferred. Administering to subjects in need of therapy for conditions that will benefit from modulation of VEGFR receptors, are particularly contemplated.

Polypeptides of the present invention that bind and inhibit VEGFR-3 or that are conjugated to a cytotoxic moiety can be used to target neoplasia characterized by cells expressing VEGFR-3 on their surfaces.

Polypeptides of the invention that can activate VEGFR-3 can be used to promote the endothelial functions of lymphatic vessels and tissues such as to treat loss of lymphatic vessels, occlusions of lymphatic vessels, lymphangiomas, and primary idiopathic lymphedemas, including Milroy's disease and lymphedema praecox, as well as secondary lymphedemas, including those resulting from removal of lymph nodes and vessels, radiotherapy and surgery in treatment of cancer, trauma and infection.

Polynucleotides or polypeptides of the invention can be administered purely as a prophylactic treatment to prevent lymphedema in subjects at risk for developing lymphedema, or as a therapeutic treatment to subjects afflicted with lymphedema, for the purpose of ameliorating its symptoms (e.g., swelling due to the accumulation of lymph).

The polynucleotides and polypeptides of the invention that activate VEGFR-3 can also be used to promote re-growth or permeability of lymphatic vessels in patients whose axillary lymphatic vessels were removed during surgical interventions in the treatment of cancer (e.g., breast cancer). Polynucleotides and polypeptides of the invention can be used to treat vascularization in, for example, organ transplant patients. A composition containing the polypeptide(s) of the invention may be directly applied to the isolated vessel segment prior to its being grafted in vivo to minimize rejection of the transplanted material and to stimulate vascularization of the transplanted materials.

Polypeptides of the invention that activate VEGF receptor activity may be used to treat wounds, surgical incisions, sores, and other indications where healing is reasonably expected to be promoted if the process of neovascularization can be induced and/or accelerated. In certain embodiments, such polypeptides can be used to improve healing of skin flaps or skin grafts following surgery as described in commonly owned, co-filed U.S. Patent Application No. 60/478,114 (Filed Jun. 12, 2003, attorney docket No. 28967/39117), and U.S. patent application Ser. No. ______ (attorney docket No. 28967/39117A), filed Jun. 14, 2004, each incorporated herein by reference.

In addition, the expression of receptors for vascular endothelial growth factors have been observed in certain progenitor cells, such as hematopoietic and/or endothelial progenitor cells, and VEGF-C has been observed to have myelopoietic activity. These observations provide an indication that polynucleotides or polypeptides according to the invention may be used to treat or prevent inflammation, infection, or immune disorders by modulating the proliferation, differentiation and maturation, or migration of immune cells or hematopoietic cells. Polynucleotides or polypeptides according to the invention may also be useful to promote or inhibit trafficking of leukocytes between tissues and lymphatic vessels and migration in and out of the thymus. See International Patent Publication No. WO 98/33917, incorporated by reference.

Polynucleotides and polypeptides of the invention can be used for stimulating myelopoiesis (especially growth of neutrophilic granuloctyes) or inhibiting it. See International Patent Publication No. WO 98/33917, incorporated by reference. Thus, the invention includes a method for modulating myelopoiesis in a mammalian subject comprising administering to a mammalian subject in need of modulation of myelopoiesis an amount of a polypeptide of the invention that is effective to modulate myelopoiesis. In one embodiment, a mammalian subject suffering from granulocytopenia is selected, and the method comprises administering to the subject an amount of a polypeptide effective to stimulate myelopoiesis. In particular, a polypeptide of the invention is administered in an amount effective to increase the neutrophil count in blood of the subject.

In a related embodiment, the invention includes a method of increasing the number of neutrophils in the blood of a mammalian subject comprising the step of expressing in a cell in a subject in need of an increased number of blood neutrophils a DNA encoding a polynucleotide of the invention that is able to activate signaling through VEGF receptors, the DNA operatively linked to a promoter or other control sequence that promotes expression of the DNA in the cell. Similarly, the invention includes a method of modulating the growth of neutrophilic granulocytes in vitro or in vivo comprising the step of contacting mammalian stem cells with a polypeptide of the invention in an amount effective to modulate the growth of mammalian endothelial cells.

The invention also includes a method for modulating the growth of CD34+ progenitor cells (especially hematopoietic progenitor cells and endothelial progenitor cells, more preferably CD34+/VEGFR-3+, still more preferably CD34+, CD133+/VEGFR3+ cells) in vitro or in vivo comprising the step of contacting mammalian CD34+ progenitor cells with a polypeptide of the invention in an amount effective to modulate the growth of mammalian endothelial cells. For in vitro methods, CD34+ progenitor cells isolated from cord blood or bone marrow are specifically contemplated. Further isolation of the CD133+ VEGFR-3+ subfraction also is contemplated. In vitro and in vivo methods of the invention for stimulating the growth of CD34+ precursor cells also include methods wherein polypeptides of the invention are employed together (simultaneously or sequentially) with other polypeptide factors for the purpose of modulating hematopoiesis/myelopoiesis or endothelial cell proliferation. Such other factors include, but are not limited to colony stimulating factors (“CSFs,” e.g., granulocyte-CSF (G-CSF), macrophage-CSF (M-CSF), and granulocyte-macrophage-CSF (GM-CSF)), interleukin-3 (IL-3, also called multi-colony stimulating factor), other interleukins, stem cell factor (SCF), other polypeptide factors, and their analogs that have been described and are known in the art. See generally The Cytokine Handbook, Second Ed., Angus Thomson (editor), Academic Press (1996); Callard and Gearing, The Cytokine FactsBook, Academic Press Inc. (1994); and Cowling and Dexter, TIBTECH, 10(10):349-357 (1992). The use of a polypeptide of the invention as a progenitor cell or myelopoietic cell growth factor or co-factor with one or more of the foregoing factors may potentiate previously unattainable myelopoietic effects and/or potentiate previously attainable myelopoietic effects while using less of the foregoing factors than would be necessary in the absence of a polypeptide of the invention.

Polynucleotides and polypeptides of the invention may also be used in the treatment of lung disorders to improve blood circulation in the lung and/or gaseous exchange between the lungs and the blood stream; to improve blood circulation to the heart and O₂ gas permeability in cases of cardiac insufficiency; to improve blood flow and gaseous exchange in chronic obstructive airway disease; and to treat conditions such as congestive heart failure, involving accumulations of fluid in, for example, the lung resulting from increases in vascular permeability, by exerting an offsetting effect on vascular permeability in order to counteract the fluid accumulation.

Polypeptides of the invention that bind but do not stimulate signaling through one or more of the VEGF receptors may be used to treat chronic inflammation caused by increased vascular permeability, retinopathy associated with diabetes, rheumatoid arthritis and psoriasis. Polynucleotides or polypeptides according to the invention that are able to inhibit the function of one or more VEGF receptors can also be used to treat edema, peripheral arterial disease, Kaposi's sarcoma, or abnormal retinal development in premature newborns.

In another embodiment, the invention provides a method for modulating the growth of endothelial cells in a mammalian subject comprising the steps of exposing mammalian endothelial cells to a polypeptide according to the invention in an amount effective to modulate the growth of the mammalian endothelial cells. In one embodiment, the modulation of growth is affected by using a polypeptide capable of stimulating tyrosine phosphorylation of VEGF receptors in a host cell expressing the VEGF receptors. In modulating the growth of endothelial cells, the invention contemplates the modulation of endothelial cell-related disorders. In a preferred embodiment, the subject, and endothelial cells, are human. The endothelial cells may be provided in vitro or in vivo, and they may be contained in a tissue graft. An effective amount of a polypeptide is an amount necessary to achieve a reproducible change in cell growth rate (as determined by microscopic or macroscopic visualization and estimation of cell doubling time, or nucleic acid synthesis assays).

Since angiogenesis and neovascularization are essential for tumor growth, inhibition of angiogenic activity can prevent further growth and even lead to regression of solid tumors. Likewise inhibition of lymphangeogenesis may be instrumental in preventing metastases. See e.g., International Publication Nos. WO 02/060950 and WO 00/21560, incorporated herein by reference. Polynucleotides and polypeptides of the invention, when conjugated to a cytotoxic agent may be, used to treat neoplasias including sarcomas, melanomas, carcinomas, and gliomas by inhibiting tumor angiogenesis.

Thus, it is contemplated that a wide variety of cancers may be treated using the peptides of the present invention including cancers of the brain (glioblastoma, astrocytoma, oligodendroglioma, ependymomas), lung, liver, spleen, kidney, lymph node, pancreas, small intestine, blood cells, colon, stomach, breast, endometrium, prostate, testicle, ovary, skin, head and neck, esophagus, bone marrow, blood or other tissue.

In many contexts, it is not necessary that the tumor cell be killed or induced to undergo normal cell death or “apoptosis.” Rather, to accomplish a meaningful treatment, all that is required is that the tumor growth be slowed to some degree or localized to a specific area and inhibited from spread to disparate sites. It may be that the tumor growth is completely blocked, however, or that some tumor regression is achieved. Clinical terminology such as “remission” and “reduction of tumor” burden also are contemplated given their normal usage. In the context of the present invention, the therapeutic effect may result from an inhibition of angiogenesis and/or an inhibition of lymphangiogenesis.

VEGF-C and VEGF-D of the VEGF family of growth factors have utility for preventing stenosis or restenosis of blood vessels. See International Patent Application No. PCT/US99/24054 (WO 00/24412), “Use of VEGF-C or VEGF-D Gene or Protein to Prevent Restenosis,” filed Oct. 26, 1999, incorporated herein by reference in its entirety. The polypeptides and polynucleotides of the invention also will have utility for these indications and can substitute for VEGF-C and VEGF-D with respect to the materials and methods described therein. Thus, in another aspect, the invention provides a method of treating a mammalian subject to prevent stenosis or restenosis of a blood vessel, comprising the step of administering to a mammalian subject in need of treatment to prevent stenosis or restenosis of a blood vessel a composition comprising one or more polypeptide(s) or polynucleotide(s) of the invention, in an amount effective to prevent stenosis or restenosis of the blood vessel. In a preferred embodiment, the administering comprises implanting an intravascular stent in the mammalian subject, where the stent is coated or impregnated with the composition. Exemplary materials for constructing a drug-coated or drug-impregnated stent are described in literature cited above and reviewed in Lincoff et al., Circulation, 90: 2070-2084 (1994). In another preferred embodiment, the composition comprises microparticles composed of biodegradable polymers such as PGLA, non-degradable polymers, or biological polymers (e.g., starch) which particles encapsulate or are impregnated by a polypeptide(s) of the invention. Such particles are delivered to the intravascular wall using, e.g., an infusion angioplasty catheter. Other techniques for achieving locally sustained drug delivery are reviewed in Wilensky et al., Trends Caridovasc. Med., 3: 163-170 (1993), incorporated herein by reference.

Administration via one or more intravenous injections subsequent to the angioplasty or bypass procedure also is contemplated. Localization of the polypeptides of the invention to the site of the procedure occurs due to expression of VEGF receptors on proliferating endothelial cells and due to heparin sulfate binding property of the molecules of the present invention. Localization is further facilitated by recombinantly expressing the polypeptides of the invention as a fusion polypeptide (e.g., fused to an apolipoprotein B-100 oligopeptide as described in Shih et al., Proc. Nat'l. Acad. Sci. USA, 87:1436-1440 (1990). Co-administration of polynucleotides and polypeptides of the invention is also contemplated.

Likewise, the invention also provides surgical devices that are used to treat circulatory disorders, such as intravascular or endovascular stents, balloon catheters, infusion-perfusion catheters, extravascular collars, elastomeric membranes, and the like, which have been improved by coating with, impregnating with, adhering to, or encapsulating within the device a composition comprising a polynucleotide or polypeptide of the invention.

Polynucleotides or polypeptides of the invention can be administered purely as a prophylactic treatment to prevent stenosis, or shortly before, and/or concurrently with, and/or shortly after a percutaneous transluminal coronary angioplasty procedure, for the purpose of preventing restenosis of the subject vessel. In another preferred embodiment, the polynucleotide or polypeptide is administered before, during, and/or shortly after a bypass procedure (e.g., a coronary bypass procedure), to prevent stenosis or restenosis in or near the transplanted (grafted) vessel, especially stenosis at the location of the graft itself. In yet another embodiment, the polynucleotide or polypeptide is administered before, during, or after a vascular transplantation in the vascular periphery that has been performed to treat peripheral ischemia or intermittent claudication. By prevention of stenosis or restenosis is meant prophylactic treatment to reduce the amount/severity of, and/or substantially eliminate, the stenosis or restenosis that frequently occurs in such surgical procedures. The polynucleotide or polypeptide is included in the composition in an amount and in a form effective to promote stimulation of VEGF receptors in a blood vessel of the mammalian subject, thereby preventing stenosis or restenosis of the blood vessel.

In a preferred embodiment, the mammalian subject is a human subject. For example, the subject is a person suffering from coronary artery disease that has been identified by a cardiologist as a candidate who could benefit from a therapeutic balloon angioplasty (with or without insertion of an intravascular stent) procedure or from a coronary bypass procedure. Practice of methods of the invention in other mammalian subjects, especially mammals that are conventionally used as models for demonstrating therapeutic efficacy in humans (e.g., primate, porcine, canine, or rabbit animals), also is contemplated.

The polypeptides of the invention may be used to modulate the growth of isolated cells or cell lines. For example, certain neoplastic disease states are characterized by the appearance of VEGF receptors on cell surfaces [Valtola et al., Am J Path 154:1381-90 (1999)]. Polypeptides of the invention may be screened to determine the ability of the polypeptide to modulate the growth of the neoplastic cells. Other disease states are likely characterized by mutations in VEGF receptors [Ferrell et al., Hum Mol Genetics 7:2073-78 (1998)]. Polypeptides of the invention that modulate the activity of the mutant forms of the VEGF receptor in a manner different than naturally-occurring vascular endothelial growth factors will be useful at modulating the symptoms and severity of such disease states.

Polypeptides of the invention may be used to modulate the growth of stem cells, progenitor cells for various tissues, and primary cell isolates that express receptor for the polypeptides.

As indicated herein above, and discussed further in U.S. patent application Ser. No. 10/669,176, filed Sep. 23, 2003, VEGF-C compositions are useful in the treatment of neurological disorders. The compositions of the invention are useful in the treatment of such disorders either alone or in conjunction with additional therapeutics, such as a neural growth factor. Exemplary neural growth factors include, but are not limited to, interferon gamma, nerve growth factor, epidermal growth factor (EGF), basic fibroblast growth factor (bFGF), neurogenin, brain derived neurotrophic factor (BDNF), thyroid hormone, bone morphogenic proteins (BMPs), leukemia inhibitory factor (LIF), sonic hedgehog, and glial cell line-derived neurotrophic factor (GDNF), vascular endothelial growth factor (VEGF), interleukins, interferons, stem cell factor (SCF), activins, inhibins, chemokines, retinoic acid and ciliary neurotrophic factor (CNTF). In one aspect, the invention contemplates a composition comprising a heparin binding VEGFR-3 ligand of the invention and a neural growth factor in a pharmaceutically acceptable diluent or carrier, or polynucleotides comprising the same.

Methods of the invention preferably are performed wherein the subject has a disease or condition characterized by aberrant growth of neuronal cells, neuronal scarring and damage or neural degeneration. A disease or medical disorder is considered to be nerve damage if the survival or function of nerve cells and/or their axonal processes is compromised. Such nerve damage occurs as the result of conditions including: physical injury, which causes the degeneration of the axonal processes and/or nerve cell bodies near the site of the injury; ischemia, as a stroke; exposure to neurotoxins, such as the cancer and AIDS chemotherapeutic agents such as cisplatin and dideoxycytidine (ddC), respectively; chronic metabolic diseases, such as diabetes or renal dysfunction; and neurodegenerative diseases such as Parkinson's disease, Alzheimer's disease, and Amyotrophic Lateral Sclerosis (ALS), which cause the degeneration of specific neuronal populations. Conditions involving nerve damage include Parkinson's disease, Alzheimer's disease, Amyotrophic Lateral Sclerosis, stroke, diabetic polyneuropathy, toxic neuropathy, glial scar, and physical damage to the nervous system such as that caused by physical injury of the brain and spinal cord or crush or cut injuries to the arm and hand or other parts of the body, including temporary or permanent cessation of blood flow to parts of the nervous system, as in stroke.

In one embodiment, the disease or condition being treated is a neurodegenerative disorder, wherein the neurodegenerative disorder is selected from the group consisting of Alzheimer's disease, Parkinson's disease, Huntington's disease, motor neuron disease, Amyotrophic Lateral Sclerosis (ALS), dementia and cerebral palsy. In another embodiment, the disease or condition is selected from the group consisting of neural trauma or neural injury. Methods of the invention also can be performed to treat or ameliorate the effects of neural trauma or injury, such as injury related to stroke, spinal cord injury, post-operative injury, brain ischemia and other traumas.

The invention can be used to treat one or more adverse consequences of central nervous system injury that arise from a variety of conditions. Thrombus, embolus, and systemic hypotension are among the most common causes of stroke. Other injuries may be caused by hypertension, hypertensive cerebral vascular disease, rupture of an aneurysm, an angioma, blood dyscrasia, cardiac failure, cardiac arrest, cardiogenic shock, kidney failure, septic shock, head trauma, spinal cord trauma, seizure, bleeding from a tumor, or other loss of blood volume or pressure. These injuries lead to disruption of physiologic function, subsequent death of neurons, and necrosis (infarction) of the affected areas. The term “stroke” connotes the resulting sudden and dramatic neurologic deficits associated with any of the foregoing injuries.

The terms “ischemia” or “ischemic episode,” as used herein, means any circumstance that results in a deficient supply of blood to a tissue. Thus, a central nervous system ischemic episode results from an insufficiency or interruption in the blood supply to any locus of the brain such as, but not limited to, a locus of the cerebrum, cerebellum or brain stem. The spinal cord, which is also a part of the central nervous system, is equally susceptible to ischemia resulting from diminished blood flow. An ischemic episode may be caused by a constriction or obstruction of a blood vessel, as occurs in the case of a thrombus or embolus. Alternatively, the ischemic episode may result from any form of compromised cardiac function, including cardiac arrest, as described above. Where the deficiency is sufficiently severe and prolonged, it can lead to disruption of physiologic function, subsequent death of neurons, and necrosis (infarction) of the affected areas. The extent and type of neurologic abnormality resulting from the injury depend on the location and size of the infarct or the focus of ischemia. Where the ischemia is associated with a stroke, it can be either global or focal in extent.

Polypeptides and polynucleotide compositions of the invention will also be useful for treating traumatic injuries to the central nervous system that are caused by mechanical forces, such as a blow to the head. Trauma can involve a tissue insult selected from abrasion, incision, contusion, puncture, compression, etc., such as can arise from traumatic contact of a foreign object with any locus of or appurtenant to the mammalian head, neck or vertebral column. Other forms of traumatic injury can arise from constriction or compression of mammalian CNS tissue by an inappropriate accumulation of fluid (e.g., a blockade or dysfunction of normal cerebrospinal fluid or vitreous humour fluid production, turnover or volume regulation, or a subdural or intracranial hematoma or edema). Similarly, traumatic constriction or compression can arise from the presence of a mass of abnormal tissue, such as a metastatic or primary tumor.

It is further contemplated that methods of the invention directed to neurological indications can be practiced by co-administering a polypeptide of the formula X-B-Z or Z-B-X with a neurotherapeutic agent. By “neurotherapeutic agent” is meant an agent used in the treatment of neurodegenerative diseases or to treat neural trauma and neural injury. Exemplary neurotherapeutic agents include tacrine (Cognex), donepezil (Aricept), rivastigmine (Exelon), galantamine (Reminyl), and cholinesterase inhibitors and anti-inflammatory drugs, which are useful in the treatment of Alzheimer's disease as well as other neurodegenerative diseases.

Additional neurotherapeutic agents include anti-cholinergics, dopamine agonists, catechol-0-methyl-transterases (COMTs), amantadine (Symmetrel), Sinemet®, Selegiline, carbidopa, ropinirole (Requip), coenzyme Q10, Pramipexole (Mirapex) and levodopa (L-dopa), which are useful in the treatment of Parkinson's disease as well as other neurodegenerative diseases. Other therapeutics agents for the treatment of neurological disorders will be known to those of skill in the art and may be useful in the combination therapies contemplated herein.

E. Pharmaceutical Compositions Comprising Chimeric VEGF Polypeptides

Polypeptides and/or polynucleotides of the invention may be administered in any suitable manner using an appropriate pharmaceutically-acceptable vehicle, e.g., a pharmaceutically-acceptable diluent, adjuvant, excipient or carrier. The composition to be administered according to methods of the invention preferably comprises (in addition to the polynucleotide or vector) a pharmaceutically-acceptable carrier solution such as water, saline, phosphate-buffered saline, glucose, or other carriers conventionally used to deliver therapeutics intravascularly. Multi-gene therapy is also contemplated, in which case the composition optionally comprises both the polynucleotide of the invention/vector and another polynucleotide/vector selected to prevent restenosis or other disorder mediated through the action of a VEGF receptor. Exemplary candidate genes/vectors for co-transfection with transgenes encoding polypeptides of the invention are described in the literature cited above, including genes encoding cytotoxic factors, cytostatic factors, endothelial growth factors, and smooth muscle cell growth/migration inhibitors.

The “administering” that is performed according to the present method may be performed using any medically-accepted means for introducing a therapeutic directly or indirectly into the vasculature of a mammalian subject, including but not limited to injections (e.g., intravenous, intramuscular, subcutaneous, or catheter); oral ingestion; intranasal or topical administration; and the like. In a preferred embodiment, administration of the composition comprising a polynucleotide of the invention is performed intravascularly, such as by intravenous, intra-arterial, or intracoronary arterial injection. The therapeutic composition may be delivered to the patient at multiple sites. The multiple administrations may be rendered simultaneously or may be administered over a period of several hours. In certain cases it may be beneficial to provide a continuous flow of the therapeutic composition. Additional therapy may be administered on a period basis, for example, daily, weekly or monthly. To minimize angiogenic side effects in non-target tissues, preferred methods of administration are methods of local administration, such as admistration by intramuscular injection.

In general, peroral dosage forms for the therapeutic delivery of polypeptides is ineffective because in order for such a formulation to the efficacious, the peptide must be protected from the enzymatic environment of the gastrointestinal tract. Additionally, the polypeptide must be formulated such that it is readily absorbed by the epithelial cell barrier in sufficient concentrations to effect a therapeutic outcome. The chimeric polypeptides of the present invention may be formulated with uptake or absorption enhancers to increase their efficacy. Such enhancer include for example, salicylate, glycocholate/linoleate, glycholate, aprotinin, bacitracin, SDS caprate and the like. An additional detailed discussion of oral formulations of peptides for therapeutic delivery is found in Fix, J. Pharm. Sci., 85(12) 1282-1285, 1996, and Oliyai and Stella, Ann. Rev. Pharmacol. Toxicol., 32:521-544, 1993, both incorporated by reference.

The amounts of peptides in a given dosage will vary according to the size of the individual to whom the therapy is being administered as well as the characteristics of the disorder being treated. In exemplary treatments, it may be necessary to administer about 50 mg/day, 75 mg/day, 100 mg/day, 150 mg/day, 200 mg/day, 250 mg/day. These concentrations may be administered as a single dosage form or as multiple doses.

The polypeptides may also be employed in accordance with the present invention by expression of such polypeptide in vivo, which is often referred to as gene therapy. The present invention provides a recombinant DNA vector containing a heterologous segment encoding a chimeric polypeptide of the invention that is capable of being inserted into a microorganism or eukaryotic cell and that is capable of expressing the encoded chimeric protein.

In a highly preferred embodiment, the composition is administered locally. Thus, in the context of treating restenosis or stenosis, administration directly to the site of angioplasty or bypass is preferred. For example, the administering comprises a catheter-mediated transfer of the transgene-containing composition into a blood vessel of the mammalian subject, especially into a coronary artery of the mammalian subject. Exemplary materials and methods for local delivery are reviewed in Lincoff et al., Circulation, 90: 2070-2084 (1994); and Wilensky et al, Trends Cardiovasc. Med., 3:163-170 (1993), both incorporated herein by reference. For example, the composition is administered using infusion-perfusion balloon catheters (preferably microporous balloon catheters) such as those that have been described in the literature for intracoronary drug infusions. See, e.g., U.S. Pat. No. 5,713,860 (Intravascular Catheter with Infusion Array); U.S. Pat. No. 5,087,244; U.S. Pat. No. 5,653,689; and Wolinsky et al., J. Am. Coll. Cardiol., 15: 475-481 (1990) (Wolinsky Infusion Catheter); and Lambert et al., Coron. Artery Dis., 4: 469-475 (1993), all of which are incorporated herein by reference in their entirety. Use of such catheters for site-directed somatic cell gene therapy is described, e.g., in Mazur et al., Texas Heart Institute Journal, 21; 104-111 (1994), incorporated herein by reference. In an embodiment where the transgene encoding a chimeric polypeptide of the invention is administered in an adenovirus vector, the vector is preferably administered in a pharmaceutically acceptable carrier at a dose of 10⁷-10¹³ viral particles, and more preferably at a dose of 10⁹-10¹¹ viral particles. The adenoviral vector composition preferably is infused over a period of 15 seconds to 30 minutes, more preferably 1 to 10 minutes.

For example, in patients with angina pectoris due to a single or multiple lesions in coronary arteries and for whom PTCA is prescribed on the basis of primary coronary angiogram findings, an exemplary protocol involves performing PTCA through a 7 F guiding catheter according to standard clinical practice using the femoral approach. If an optimal result is not achieved with PTCA alone, then an endovascular stent also is implanted. (A nonoptimal result is defined as residual stenosis of >30% of the luminal diameter according to a visual estimate, and B or C type dissection.) Arterial gene transfer at the site of balloon dilatation is performed with a replication-deficient adenoviral vector expressing a polypeptide of the invention immediately after the angioplasty, but before stent implantation, using an infusion-perfusion balloon catheter. The size of the catheter will be selected to match the diameter of the artery as measured from the angiogram, varying, e.g., from 3.0 to 3.5 F in diameter. The balloon is inflated to the optimal pressure and gene transfer is performed during a 10 minute infusion at the rate of 0.5 ml/min with virus titer of 1.15×10¹⁰ pfu/ml.

In another embodiment, intravascular administration with a gel-coated catheter is contemplated, as has been described in the literature to introduce other transgenes. See, e.g., U.S. Pat. No. 5,674,192 (Catheter coated with tenaciously-adhered swellable hydrogel polymer); Riessen et al., Human Gene Therapy, 4: 749-758 (1993); and Steg et al., Circulation, 96: 408-411 (1997) and 90: 1648-1656 (1994); all incorporated herein by reference. Briefly, DNA in solution (e.g., a polynucleotide of the invention) is applied one or more times ex vivo to the surface of an inflated angioplasty catheter balloon coated with a hydrogel polymer (e.g., Slider with Hydroplus, Mansfield Boston Scientific Corp., Watertown, Mass.). The Hydroplus coating is a hydrophilic polyacrylic acid polymer that is cross-linked to the balloon to form a high molecular weight hydrogel tightly adhered to the balloon. The DNA covered hydrogel is permitted to dry before deflating the balloon. Re-inflation of the balloon intravascularly, during an angioplasty procedure, causes the transfer of the DNA to the vessel wall.

In yet another embodiment, an expandable elastic membrane or similar structure mounted to or integral with a balloon angioplasty catheter or stent is employed to deliver the transgene encoding a polypeptide of the invention. See, e.g., U.S. Pat. Nos. 5,707,385, 5,697,967, 5,700,286, 5,800,507, and 5,776,184, all incorporated by reference herein.

In another variation, the composition containing the transgene encoding a polypeptide of the invention is administered extravascularly, e.g., using a device to surround or encapsulate a portion of vessel. See, e.g., International Patent Publication WO 98/20027, incorporated herein by reference, describing a collar that is placed around the outside of an artery (e.g., during a bypass procedure) to deliver a transgene to the arterial wall via a plasmid or liposome vector.

In still another variation, endothelial cells or endothelial progenitor cells are transfected ex vivo with the transgene encoding a polypeptide of the invention, and the transfected cells as administered to the mammalian subject. Exemplary procedures for seeding a vascular graft with genetically modified endothelial cells are described in U.S. Pat. No. 5,785,965, incorporated herein by reference.

In preferred embodiments, polynucleotides of the invention further comprises additional sequences to facilitate the gene therapy. In one embodiment, a “naked” transgene encoding a polypeptide of the invention (i.e., a transgene without a viral, liposomal, or other vector to facilitate transfection) is employed for gene therapy. In this embodiment, the polynucleotide of the invention preferably comprises a suitable promoter and/or enhancer sequence (e.g., cytomegalovirus promoter/enhancer [Lehner et al., J. Clin. Microbiol., 29:2494-2502 (1991); Boshart et al., Cell, 41:521-530 (1985)]; Rous sarcoma virus promoter [Davis et al., Hum. Gene Ther., 4:151 (1993)]; Tie promoter [Korhonen et al., Blood, 86(5): 1828-1835 (1995)]; or simian virus 40 promoter) for expression in the target mammalian cells, the promoter being operatively linked upstream (i.e., 5′) of the polypeptide-coding sequence. The polynucleotides of the invention also preferably further includes a suitable polyadenylation sequence (e.g., the SV40 or human growth hormone gene polyadenylation sequence) operably linked downstream (i.e., 3′) of the polypeptide-coding sequence. The polynucleotides of the invention also preferably comprise a nucleotide sequence encoding a secretory signal peptide fused in-frame with the polypeptide sequence. The secretory signal peptide directs secretion of the polypeptide of the invention by the cells that express the polynucleotide, and is cleaved by the cell from the secreted polypeptide. The signal peptide sequence can be that of another secreted protein, or can be a completely synthetic signal sequence effective to direct secretion in cells of the mammalian subject.

The polynucleotide may further optionally comprise sequences whose only intended function is to facilitate large-scale production of the vector, e.g., in bacteria, such as a bacterial origin of replication and a sequence encoding a selectable marker. However, in a preferred embodiment, such extraneous, sequences are at least partially cleaved off prior to administration to humans according to methods of the invention. One can manufacture and administer such polynucleotides for gene therapy using procedures that have been described in the literature for other transgenes. See, e.g., Isner et al., Circulation, 91: 2687-2692 (1995); and Isner et al., Human Gene Therapy, 7: 989-1011 (1996); incorporated herein by reference in the entirety.

Any suitable vector may be used to introduce the transgene encoding one of the polypeptides of the invention, into the host. Exemplary vectors that have been described in the literature include replication-deficient retroviral vectors, including but not limited to lentivirus vectors [Kim et al., J. Virol., 72(1): 811-816 (1998); Kingsman & Johnson, Scrip Magazine, October, 1998, pp. 43-46.]; adeno-associated viral vectors [U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479; Gnatenko et al., J. Investig. Med., 45: 87-98 (1997)]; adenoviral vectors [See, e.g., U.S. Pat. No. 5,792,453; U.S. Pat. No. 5,824,544; U.S. Pat. No. 5,707,618; U.S. Pat. No. 5,693,509; U.S. Pat. No. 5,670,488; U.S. Pat. No. 5,585,362; Quantin et al., Proc. Natl. Acad. Sci. USA, 89: 2581-2584 (1992); Stratford-Perricadet et al., J. Clin. Invest., 90: 626-630 (1992); and Rosenfeld et al., Cell, 68: 143-155 (1992)]; an adenoviral-adenoassociated viral chimeric (see for example, U.S. Pat. No. 5,856,152) or a vaccinia viral or a herpesviral (see for example, U.S. Pat. No. 5,879,934; U.S. Pat. No. 5,849,571; U.S. Pat. No. 5,830,727; U.S. Pat. No. 5,661,033; U.S. Pat. No. 5,328,688; Lipofectin-mediated gene transfer (BRL); liposomal vectors [See, e.g., U.S. Pat. No. 5,631,237 (Liposomes comprising Sendai virus proteins)]; and combinations thereof. All of the foregoing documents are incorporated herein by reference in their entirety. Replication-deficient adenoviral vectors constitute a preferred embodiment.

Other non-viral delivery mechanisms contemplated include calcium phosphate precipitation (Graham and Van Der Eb, Virology, 52:456-467, 1973; Chen and Okayama, Mol. Cell Biol., 7:2745-2752, 1987; Rippe et al., Mol. Cell Biol., 10:689-695, 1990) DEAE-dextran (Gopal, Mol. Cell Biol., 5:1188-1190, 1985), electroporation (Tur-Kaspa et al., Mol. Cell Biol., 6:716-718, 1986; Potter et al., Proc. Nat. Acad. Sci. USA, 81:7161-7165, 1984), direct microinjection (Harland and Weintraub, J. Cell Biol., 101:1094-1099, 1985.), DNA-loaded liposomes (Nicolau and Sene, Biochim. Biophys. Acta, 721:185-190, 1982; Fraley et al., Proc. Natl. Acad. Sci. USA, 76:3348-3352, 1979; Felgner, Sci Am. 276(6):102-6, 1997; Felgner, Hum Gene Ther. 7(15):1791-3, 1996), cell sonication (Fechheimer et al., Proc. Natl. Acad. Sci. USA, 84:8463-8467, 1987), gene bombardment using high velocity microprojectiles (Yang et al., Proc. Natl. Acad. Sci USA, 87:9568-9572, 1990), and receptor-mediated transfection (Wu and Wu, J. Biol. Chem., 262:4429-4432, 1987; Wu and Wu, Biochemistry, 27:887-892, 1988; Wu and Wu, Adv. Drug Delivery Rev., 12:159-167, 1993).

The expression construct (or indeed the polypeptides discussed above) may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, In: Liver diseases, targeted diagnosis and therapy using specific receptors and ligands, Wu G, Wu C ed., New York: Marcel Dekker, pp. 87-104, 1991). The addition of DNA to cationic liposomes causes a topological transition from liposomes to optically birefringent liquid-crystalline condensed globules (Radler et al., Science, 275(5301):810-4, 1997). These DNA-lipid complexes are potential non-viral vectors for use in gene therapy and delivery.

Liposome-mediated nucleic acid delivery and expression of foreign DNA in vitro has been successful. Also contemplated in the present invention are various commercial approaches involving “lipofection” technology. In certain embodiments of the invention, the liposome may be complexed with a hemagglutinating virus (HVJ). This has been shown to facilitate fusion with the cell membrane and promote cell entry of liposome-encapsulated DNA (Kaneda et al., Science, 243:375-378, 1989). In other embodiments, the liposome may be complexed or employed in conjunction with nuclear nonhistone chromosomal proteins (HMG-1) (Kato et al., J. Biol. Chem., 266:3361-3364, 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems that can be employed to deliver a nucleic acid encoding a therapeutic gene into cells include receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993, supra).

In other embodiments, the delivery vehicle may comprise a ligand and a liposome. For example, Nicolau et al. (Methods Enzymol., 149:157-176, 1987) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also may be specifically delivered into a particular cell type by any number of receptor-ligand systems with or without liposomes.

In another embodiment of the invention, the expression construct may simply consist of naked recombinant DNA or plasmids. Transfer of the construct may be performed by any of the methods mentioned above that physically or chemically permeabilize the cell membrane. This is applicable particularly for transfer in vitro, however, it may be applied for in vivo use as well. Dubensky et al. (Proc. Nat. Acad. Sci. USA, 81:7529-7533, 1984) successfully injected polyomavirus DNA in the form of CaPO₄ precipitates into liver and spleen of adult and newborn mice demonstrating active viral replication and acute infection. Benvenisty and Neshif (Proc. Nat. Acad. Sci. USA, 83:9551-9555, 1986) also demonstrated that direct intraperitoneal injection of CaPO₄ precipitated plasmids results in expression of the transfected genes.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., Nature, 327:70-73, 1987). Several devices for accelerating small particles have been developed. One such device relies on a high voltage discharge to generate an electrical current, which in turn provides the motive force (Yang et al., Proc. Natl. Acad. Sci USA, 87:9568-9572, 1990). The microprojectiles used have consisted of biologically inert substances such as tungsten or gold beads.

In embodiments employing a viral vector, preferred polynucleotides still include a suitable promoter and polyadenylation sequence as described above. Moreover, it will be readily apparent that, in these embodiments, the polynucleotide further includes vector polynucleotide sequences (e.g., adenoviral polynucleotide sequences) operably connected to the sequence encoding a polypeptide of the invention.

Thus, in one embodiment the composition to be administered comprises a vector, wherein the vector comprises a polynucleotide of the invention. In a preferred embodiment, the vector is an adenovirus vector. In a highly preferred embodiment, the adenovirus vector is replication-deficient, i.e., it cannot replicate in the mammalian subject due to deletion of essential viral-replication sequences from the adenoviral genome. For example, the inventors contemplate a method wherein the vector comprises a replication-deficient adenovirus, the adenovirus comprising the polynucleotide of the invention operably connected to a promoter and flanked on either end by adenoviral polynucleotide sequences.

Similarly, the invention includes kits which comprise compounds or compositions of the invention packaged in a manner which facilitates their use to practice methods of the invention. In a simplest embodiment, such a kit includes a compound or composition described herein as useful for practice of the invention (e.g., polynucleotides or polypeptides of the invention), packaged in a container such as a sealed bottle or vessel, with a label affixed to the container or included in the package that describes use of the compound or composition to practice the method of the invention. Preferably, the compound or composition is packaged in a unit dosage form. In another embodiment, a kit of the invention includes a composition of both a polynucleotide or polypeptide packaged together with a physical device useful for implementing methods of the invention, such as a stent, a catheter, an extravascular collar, a polymer film, or the like. In another embodiment, a kit of the invention includes compositions of both a polynucleotide or polypeptide of the invention packaged together with a hydrogel polymer, or microparticle polymers, or other carriers described herein as useful for delivery of the polynucleotides or polypeptides to the patient.

### EXAMPLE 1

### Recombinant VEGF-C with Heparin Binding Property

The present Example describes the generation of chimeric VEGF-C molecules comprising an amino terminal VEGFR-3 binding domain of VEGF-C fused to a carboxy terminal heparin binding domain from VEGF. These molecules retain VEGFR-3 binding activity as shown by a cell survival assay and are expected to have an enhanced heparin binding activity as compared to native VEGF-C and enhanced angiogenic and/or lymphangiogenic properties.

Materials & Methods

Cloning: cDNAs encoding the fusion proteins comprised of the VEGF homology domain of VEGF-C and the C-terminus of VEGF (exon 6-8 encoded polypeptide fragment, referred to below as CA89, or exon 6-7 encoded fragment referred to below as CA65) were constructed by PCR amplification using the following primers: VEGF-CΔNΔC, 5′-ACATTGGTGTGCACCTCCAAGC-3′ (SEQ ID NO: 16) and 5′-AATAATGGAATGAACTTGTCTGTAAAC-3′ (SEQ ID NO: 17); VEGF C-terminal regions: 5′-AAATCAGTTCGAGGAAAGGGAAAG-3′ (SEQ ID NO: 18) or 5′-CCCTGTGGGCCTTGCTCAGAG-3′ (SEQ ID NO: 19), and 5′-CCATGCTCGAGAGTCTTTCCTGGTGAGAGATCTGG-3′ (SEQ ID NO: 20). The PCR products were digested with HindIII (5′-HindIII/3′-blunt) or XhoI (5′-blunt-3′-XhoI), and cloned into the pEBS7 (Peterson and Legerski, Gene, 107 279-84, 1991)) expression vector that had been digested with the same enzymes to create clones named pEBS7/CA89 and pEBS7/CA65. The inserts were also subcloned into pREP7 at HindIII/XhoI sites (pREP7/CA89 and pREP7/CA65.

Cell culture, transfection and immunoprecipitation: 293T and 293EBNA cells were maintained in DMEM medium supplemented with 2 mM L-glutamine, penicillin (100 U/ml), streptomycin (100 μg/ml), and 10% fetal bovine serum (Autogen Bioclear). BaF3 cells (Achen et al., Eur J Biochem., 267: 2505-15, 2000) were grown in DMEM as above with the addition of Zeocine (200 μg/ml) and the recombinant human VEGF-CΔNΔC (100 ng/ml).

293T cells were transfected with pEBS7/CA89, pEBS7/CA65 or the pEBS7 vector using liposomes (FuGENE 6, Roche). Cells transfected with pEBS7/CA89 were cultured with or without heparin (20 unit/ml). Transfected cells were cultured for 24 h, and were then metabolically labeled in methionine-free and cysteine-free modified Eagle medium supplemented with [³⁵S]methionine/[³⁵S]cysteine (Promix, Amersham Pharmacia Biotech) at 100 μCi/mL for 8 h. Conditioned medium was then harvested, cleared of particulate material by centrifugation, and incubated with polyclonal antibodies against VEGF-C [Joukov et al., EMBO J. 16:3898-911, 1997). The formed antigen-antibody complexes were bound to protein A Sepharose (Pharmacia Biotech), which were then washed twice with 0.5% bovine serum albumin/0.02% Tween 20 in phosphate-buffered saline (PBS) and once with PBS, and analysed in sodium dodecyl sulfate-polyacrilamide gel electrophoresis (SDS-PAGE) under reducing conditions.

293EBNA cells were transfected with pREP7/CA89, pREP7/CA65 or the pREP7 vector as described above. Cells transfected with pREP7/CA89 were cultured with or without heparin (20 unit/ml). The transfected cells were cultured for two days, and the supernatants were harvested for the assay of biological activity.

Bioassay for growth factor-mediated cell survival: Ba/F3 cells expressing a VEGFR-3/EpoR chimeric receptor (Achen et al., Eur J Biochem., 267: 2505-15, 2000) were seeded in 96-well plates at 15,000 cells/well in triplicates supplied with conditioned medium (0, 1, 5, 10 or 20 μl) from cell cultures transfected with pREP7/CA89, pREP7/CA65 or the pREP7 vector. Cell viability was measured by a colorimetric assay. MTT (3-(4,5-dimethylthiazol-2-yl)-2,5-diphenyl tetrazolium bromide (Sigma), 0.5 mg/ml) was added into each well and incubated for 4 h at 37° C. The reaction was terminated by adding 100 μl of lysis buffer (10% SDS, 10 mM HCl), and the resulting formazan products were solubilized overnight at 37° C. in a humid atmosphere. The absorbance at 540 nm was measured with a Multiscan microtiter plate reader (Labsystems).

Results & Discussion

The innate heparin binding property of certain growth factors has been implicated as important for their biological activities (Dougher et al., Growth Factors, 14: 257-68, 1997; Carmeliet et al., Nat Med 5: 495-502, 1999; Ruhrberg et al., Genes Dev 16 2684-98, 2002). VEGF (Poltorak et al., J. Biol. Chem. 272:7151-8, 1997; Gitay-Goren et al., J Biol Chem 271: 5519-23, 1996), VEGF-B₁₆₇ (Makinen et al., J. Biol. Chem., 274:21217-22, 1999), and PlGF-2 (Hauser and Weich, Growth Factors, 9 259-68, 1993) all possess significant heparin binding activity, but VEGF-C and VEGF-D do not. Both of these latter molecules have been shown to induce lymphangiogenesis in transgenic mice and in other in vivo models (Jeltsch et al., Science 276:1423-5, 1997; Oh et al., Dev Biol 188: 96-109, 1997; Veikkola et al., EMBO J 20: 1223-31, 2001). Although recombinant proteins of mature forms of VEGF-C and VEGF-D are believed to exert angiogenic activity via VEGFR-2 (Cao et al. Proc Natl Acad Sci USA 95: 14389-94, 1998; Marconcini et al., Proc Natl Acad Sci USA 96: 9671-6, 1999), mature forms of VEGF-C delivered by other means such as adenoviral vectors have so far induced weak lymphangiogenic activity and little, if any, angiogenic activity in mice. These data suggest that the concentration of the protein present may not be sufficient, or that the half-life of the mature form of VEGF-C protein may be too short to induce a potent angiogenic effect. Maximum activation of VEGFR-2 in vivo may also require the ligand to have the property of heparin binding, as suggested for VEGF (Dougher et al., Growth Factors, 14: 257-68, 1997).

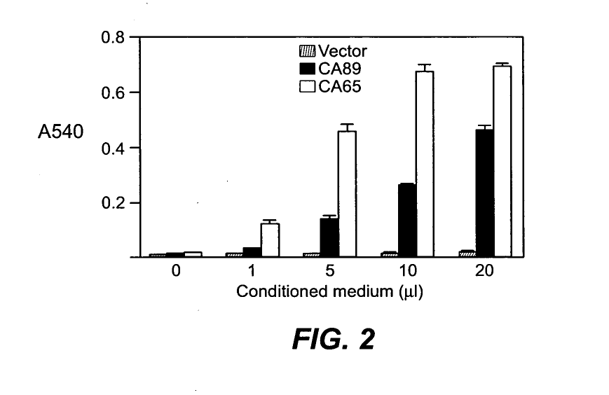

To investigate the effects of introducing a heparin binding activity on the angiogenic and lymphangiogenic effects of VEGF-C, plasmids encoding chimeric proteins comprised of the signal sequence and the VEGF homology domain (VHD) of VEGF-C, and VEGF exon 6-8 or exon 7-8 encoded sequences (FIG. 1C) were constructed. Expression of the chimeric VEGF-C proteins by the transfected cells was confirmed by immunoprecipitation with polyclonal antibodies against VEGF-C (FIG. 1D). CA65 was secreted and released into the supernatant, but CA89 was not released into the supernatant unless heparin was included in the culture medium (FIG. 1D), indicating that it apparently binds to cell surface heparan sulfates similar to what has been described for VEGF₁₈₉. VEGFR-3-mediated biological activity of the chimeric proteins (CA89 and CA65) was demonstrated by a bioassay using Ba/F3 cells expressing a chimeric VEGFR-3/erythropoietin (Epo) receptor (Ba/F3/VEGFR-3). Conditioned medium from both 293EBNA/CA89 and 293EBNA/CA65 cells were shown to induce survival and proliferation of the IL-3 dependent Ba/F3/VEGFR-3 cells in the absence of the recombinant IL-3 protein (FIG. 2). The effect was detectable even with 1 μl of the conditioned medium added.

Lymphatic vessels typically accompany blood vessels. The chimeric molecules of the present invention may allow efficient localization of growth factors expressed in a given tissue, without the danger of obtaining aberrant side effects in other sites/organs. Secondly, the heparin binding forms would allow a growth factor gradient to be established for vessel sprouting. Further, given the teachings described herein, the chimeric polypeptides of the present invention which are heparin binding factors give enhanced lymphangiogenic and/or angiogenic effects, as their three dimensional diffusion is replaced by two-dimensional diffusion in the plane of the cell surface heparin matrix, which leads to a more concentrated form of the growth factor available for the high-affinity signal transducing receptors. Furthermore, heparin binding forms of VEGF containing the VEGF exon 7-encoded sequence can also bind to neuropilins, which have important roles in the development of the cardiovascular system and the lymphatic system. Consequently, the putative neuropilin-1 binding property of the chimeric polypeptides of the invention could direct VEGF-C towards more efficient stimulation of angiogenesis.

### EXAMPLE 2

### VEGF-C Fused to Heparin-Binding Domain has Increased Lymphangiogenic Activity

The present example further demonstrates that chimeric VEGF-C molecules containing a heparin binding domain have increased lymphangiogenic activity ,in comparison with the VEGF-CΔNΔC form. The enhancement of the biological activity may result from an increased bioavailabiiity of the protein, or increased receptor binding via binding to NP-1 or NP-2. Without being bound to any theory of mechanism of action, it is possible that the presence of the heparin binding domain facilitates a two-dimensional diffusion of the heparin-domain-containing chimeric VEGF-C molecules such that the chimeric molecules become distributed in the plane of the cell surface heparan sulphate matrix, which leads to a more concentrated form of the growth factor presented and available for the high-affinity signal-transducing receptors. Furthermore, the heparin binding forms may allow a growth factor gradient to be established for vessel sprouting.

Materials and Methods

The methods described in Example 1 are incorporated into the present Example by reference. The studies described in the present example also employed the following additional experimental protocols.

Production and in vivo delivery of CA89 and CA65 by viral vectors. The AAV vector psub-CAG-WPRE was cloned by substituting the CMV promoter fragment of psub-CMV-WPRE (Paterna et al., Gene Ther., 7(15):1304-1311, 2000) with the CMV-chicken beta-actin insert (Niwa et al., Gene, 108(2):193-199, 1991). The cDNAs encoding CA89 and CA65 were cloned as blunt-end fragments into the psub-CAG-WPRE plasmid, and the recombinant AAV viruses (AAV.CA89 and AAV.CA65, AAV serotype 2) were produced as described in Karkkainen et al., Proc. Natl. Acad. Sci. USA, 98(22):12677-12682 (2001). The cDNAs encoding CA89 and CA65 were also cloned into the pAdBglII vector (AdCA89 and AdCA65), and recombinant adenoviruses were produced as described in Laitinen et al., Hum. Gene Ther., 9(10):1481-1486, 1998. NCI-H460-LNM35 cells (Kozaki et al., Cancer Res., 60(9):2535-2540, 2000) were used for expression analysis. These cells were maintained in RPMI1640 medium with supplements (2 mM L-glutamine, penicillin 100 U/ml, streptomycin 100 μg/ml, and 10% fetal bovine serum) as above and were infected with AAV.CAG.VEGFR-3-Ig viruses (MOI 2000), or adenoviruses (MOI 50). Expression of the recombinant proteins were examined by metabolic labeling, immunoprecipitation followed by SDS-PAGE analysis as described above.

Adenoviruses (AdCA89 or AdCA65, approximately 3×10⁸ pfu), or AAV viruses (AAV.CA89, AAV.CA65 or AAV.EGFP, approximately 1×10¹⁰ viral particles) were injected subcutaneously into mouse ears. Tissues were collected for analysis after two weeks with adenoviruses and three weeks with AAV viruses for histological analysis.

Fluorescent microlymphography. The functional lymphatic network in the ears was visualized by fluorescent microlymphography using dextran conjugated with fluorescein isothiocyanate (molecular weight: 2000 kDa, Sigma) that was injected intradermally into the ears. The lymphatic vessels were examined using a dissection microscope (LEICA MZFLIII).

Immunohistochemistry. For whole mount staining, tissues were fixed in 4% paraformaldehyde (PFA), blocked with 3% milk in PBS, and incubated with polyclonal antibodies against LYVE-1 (Prevo et al., J. Biol. Chem., 276(22):19420-12930, 2001) and PECAM-1 (PharMingen) overnight at 4° C. Alexa594 and Alexa488 conjugated secondary antibodies (Molecular Probes) were used for staining, and samples were then mounted with Vectashield (Vector Laboratories) and analysed with a Zeiss LSM510 confocal microscope. For staining of tissue sections, tissues were fixed in 4% PFA overnight at 4° C. and paraffin sections (6 μm) were immunostained with anti-LYVE-1 and monoclonal antibodies against PECAM-1.

Results and Discussion

As discussed in Example 1, the heparin binding property of growth factors is important in the biological activities of those factors that bind heparin. The data shown in Example 1 demonstrated that the presence of a heparin binding domain have an enhanced heparin binding activity as compared to native VEGF-C, and they are biologically active. The following discussion further corroborates those findings.

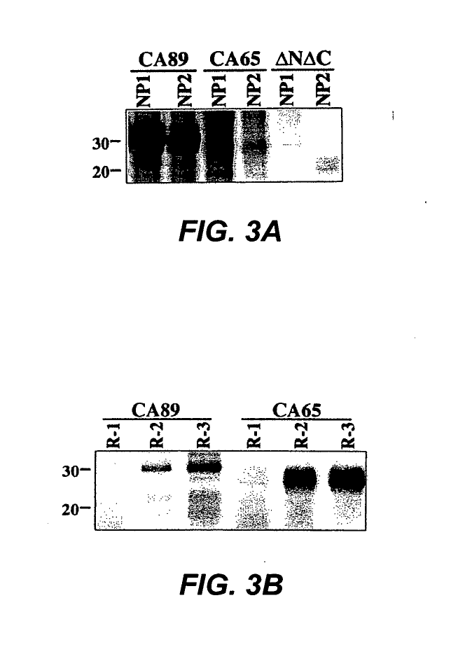

Enhancement of receptor binding activity of recombinantly processed VEGF-C by addition of heparin binding domain. Analysis of the receptor binding profiles of the chimeric molecules showed that, similar to VEGF-CΔNΔC, both CA89 and CA65 bound to VEGFR-2, VEGFR-3, but not VEGFR-1 (FIG. 3B). Heparin binding forms of VEGF, containing the VEGF exon 7-encoded sequence, have been shown to bind to neuropilins, which have important roles in the development of the cardiovascular and lymphatic systems (Soker et al., J. Biol. Chem., 271(10):5761-5767, 1996; Neufeld et al., Trends Cardiovasc. Med., 12(1):13-19, 2002). In agreement with these data, both CA89 and CA65 bound to NP-1 and NP-2, whereas VEGF-CΔNΔC had a weak binding activity to NP-2 but did not bind to NP-1 (FIG. 3A).

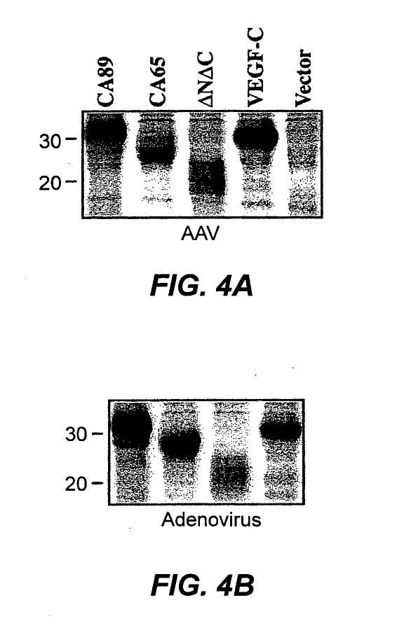

Lymphangiogenic activity of VEGF-CΔNΔC is enhanced by heparin/neuropilin binding domain. To further characterize the biological functions of the chimeric proteins in vivo, the cDNAs encoding CAS9 and CA65 were cloned into the pAdBglII vector (AdCA89 and AdCA65) for the generation of recombinant adenoviruses. Recombinant AAV (AAV.CA89 and AAV.CA65, serotype 2) were also produced to study the effect of longer-term expression of the chimeric molecules. Shown in FIG. 4 is the analysis of polypeptides produced via the AAV (FIG. 4A) and adenoviral (FIG. 4B) expression of CA89, CA65, VEGF-C and the VEGF-CΔNΔC.

For analysis of their in vivo vascular effects, adenoviruses encoding CA89, CA65, and VEGF-CΔNΔC were injected subcutaneously into the ears of nude mice. AdVEGF-C (fill length/“prepro-VEGF-C”) and AdLacZ viruses were used as positive and negative controls. Tissues were collected for whole mount immunostaining of lymphatic vessels (LYVE-1 antigen) and blood vessels (PECAM-1) within two weeks. Both CA89 and CA65 were shown to induce strong lymphangiogenesis in comparison with the LacZ control. While CA89 exerted a localized effect around the virus injection site, CA65 induced a widespread effect in a fashion similar to the full-length VEGF-C. This is in agreement with the differential distribution of the two chimeric molecules between pericellular matrix and fluid phases in culture. VEGF-CΔNΔC induced only a weak lymphangiogenic effect with some lymphatic sprouting from the pre-existing lymphatic vessels. There was no angiogenic effect observed with the heparin binding chimeric molecules, VEGF-CΔNΔC or full length VEGF-C in comparison with the control.

Both CA89 and CA65 delivered by the recombinant AAV viruses also induced strong lymphangiogenesis when compared with the control involving AAV.EGFP. However, the effects observed with AAV vectors were seen only around the ear muscles, as AAV viruses mainly transduce muscle and neurons (Daly, Methods Mol. Biol., 246:157-165, 2004). The lymphatic vessels grew along the muscle fibers that were transduced with AAV.EGFP. These data indicate that by use of a vector/tissue-specific promoter and a heparin-binding growth factor, one can achieve a more defined localization of growth factor expression in a given tissue, and therefore minimize the danger of obtaining aberrant side effects from other sites.

However, analysis by microlymphography showed that the lymphatic vessels generated in the mice receiving CA89 or CA65 via viral vectors were leaky compared with the control. Similar findings have been reported for vessels generated with full-length VEGF-C. A combination of CA89 or CA65 with other molecules such as Ang-1 is contemplated for the optimal induction of functional lymphatic vessels.

In histological sections from the AAV.CA89 treated mice, many LYVE-1-positive vessel-like structures were observed in regions close to cartilage where the ear muscles are located, whereas only a few lymphatic vessels were found in corresponding sections from the control mice. PECAM-1, a panendothelial marker for blood and lymphatic vessels, also detected more vessels in the sections from the AAV.CA89 treated mice. Similarly, many LYVE-1-positive vessel-like structures, often in clusters close to the cartilage, were found in the AAV.CA65 treated mice. In contrast, fewer lymphatic vessels were observed in the control mice.

In summary, these experiments show the lymphangiogenic and/or angiogenic properties of VEGF-C short form in the presence and absence of a heparin binding property. Chimeric proteins made of the signal sequence and the VEGF homology domain (VHD) of VEGF-C, and the C-terminal domain of VEGF165 or VEGF189 isoforms containing heparin and neuropilin1 binding sequences (named CA89 and CA65) were studied. CA65 was secreted and released into the supernatant, but CA89 was only released if heparin was included in the culture medium. Analysis of the receptor binding profiles of the chimeric molecules showed that they retained VEGFR-2 and VEGFR-3 binding and activation and in addition are expected to bind to NP-1, whereas the VEGF-C short form did not retain these binding activities. In vivo expression of the chimeric proteins delivered via adenoviral or associated virus vectors demonstrated that they induced strong lymphangiogenesis in a mouse ear model, whereas significant angiogenic activity was not observed. The enhanced lymphangiogenic activity may result from the increase of its bioavailability and/or neuropilin binding property.

### EXAMPLE 3

### Methods of Using the Chimeric Polypeptides

The heparin binding VEGFR-3 binding ligands of the invention have utility in any and all indications for which VEGF-C and/or VEGF-D are useful, as well as additional indications for which these native VEGFR-3 ligands have shown limited or no efficacy. CA89 and CA65 are only two specific exemplary embodiments of the class of chimeric polypeptides of the present invention.

The activity of chimeric polypeptides of the present invention can be demonstrated in any of a number of assays. Examples of some of these assays are discussed below. To assess comparative/relative activities, these assays are conducted in parallel with molecules of the invention and with VEGF-A, VEGF-B, VEGF-C, VEGF-C₁₅₆ mutants or fragments of a molecule of the invention containing only the VEGFR-3 binding domain or only the heparin binding domain.

Receptor Binding Assays

In a first battery of assays, one determines the receptor binding activity of the chimeric polypeptides of the present invention. It will be appreciated that such binding assays can be performed with any form of naturally occurring VEGF receptors that retain the ability to bind their respective ligands, including but not limited to whole cells that naturally express a receptor or that have been recombinantly modified to express the receptor; truncated, solubilized extracellular ligand binding domains of receptors; fusions comprising receptor extracellular domains fused to other proteins such as alkaline phosphatase (e.g., VEGF-R-2-AP described in Cao et al., J. Biol. Chem. 271:3154-62 (1996)) or immunoglobulin sequences; and fusions comprising receptor extracellular domains fused to tag sequences (e.g., a polyhistidine tag) useful for capturing the protein with an antibody or with a solid support; and receptor extracellular domains chemically attached to solid supports such as CNBr-activated Sepharose beads. Exemplary receptor binding assays may be performed according to the method set forth in Example 3 of e.g., U.S. patent application Ser. No. 09/795,006, and WO 01/62942, each incorporated herein by reference.

Analysis of Receptor Activation or Inhibition by the Chimeric VEGF Proteins

In another set of assays, the chimeric polypeptides of the present invention are evaluated for therapeutic applications where either activation or inhibition of one or more VEGF receptors is desired. For example, a candidate chimeric protein can be added to stable cell lines expressing a particular VEGF receptor whose activation is necessary for cell survival. Survival of the cell line indicates that the candidate chimeric polypeptide protein is able to bind and activate that particular VEGF receptor. On the other hand, death of the cell line indicates that the candidate chimeric polypeptide fails to activate the receptor. Exemplary examples of such cell survival assays have been described in International Patent Publication No. WO 98/07832 and in Achen et al., Proc Natl Acad Sci USA 95:548 553 (1998), incorporated herein by reference. This assay employs Ba/F3 NYK EpoR cells, which are Ba/F3 pre B cells that have been transfected with a plasmid encoding a chimeric receptor consisting of the extracellular domain of VEGFR-2 and the cytoplasmic domain of the erythropoietin receptor (EpoR). These cells are routinely passaged in interleukin-3 (IL-3) and will die in the absence of IL-3. However, if signaling is induced from the cytoplasmic domain of the chimeric receptor, these cells survive and proliferate in the absence of IL-3. Such signaling is induced by ligands which bind to the VEGFR-2 extracellular domain of the chimeric receptor. For example, binding of VEGF A or VEGF-D to the VEGFR-2 extracellular domain causes the cells to survive and proliferate in the absence of IL-3. Parental Ba/F3 cells which lack the chimeric receptor are not induced by either VEGF A or VEGF-D to proliferate in the absence of IL-3, indicating that the responses of the Ba/F3-NYK-EpoR cells to these ligands are totally dependent on the chimeric receptor.

Candidate chimeric polypeptides of the present invention can be tested for binding to the VEGFR-2 extracellular domain and subsequent activation of the chimeric receptor by assaying cell survival in the absence of IL 3. On the other hand, chimeric polypeptides that interfere with the binding of VEGFR-2 ligands, such as VEGF-A or VEGF-D, to the extracellular domain, or with the activation of the cytoplasmic domain, will cause cell death in the absence of IL-3.

Typically, in these assays, cells are cultured in the presence of IL-3 until required, then washed three times in phosphate buffered saline (PBS), resuspended in IL-3-free cell culture medium (Dulbecco's Modified Eagle's Medium (DMEM) supplemented with fetal calf serum (10%), L-glutamine (1%), geneticin (1 mg/ml), streptomycin (100 μg/ml) and penicillin (60 μg/ml)), and replated in 72-well culture plates (Nunc, Denmark) at a density of approximately 1000 cells/well. To assay for receptor activity, candidate chimeric polypeptides are added to culture wells at final concentrations of 10-10 to 10-5 M and incubated for 1 hour at 37° C. in 10% CO₂. For assaying the ability of the candidate chimeric polypeptides to inhibit activation of the VEGFR-2/EpoR receptor, recombinant VEGF A or VEGF-D is added to the chimeric polypeptide-containing wells at a concentration to produce near-maximal survival of the Ba/F3 NYK EpoR cells (typically 500 ng/ml). Positive control cultures contain either VEGF-A or VEGF-D supernatant alone and negative control cultures contain neither chimeric polypeptide nor growth factor. Cells are then grown in culture for 48 hours, after which time a solution of 3-(3,4-dimethylthiazol-2-yl)-2,5-diphenyltetrazolium bromide (MTT; 500 μg/ml) is added to the cultures, and incubated for another 30 minutes. MTT is converted to a blue formazan product by mitochondria, thus staining living cells blue. Surviving blue cells in experiments where either activation (chimeric polypeptide alone) or inhibition (chimeric polypeptide+VEGF-A or VEGF-D) was assayed are counted under a microscope with inverted optics (100× magnification) and compared to cell survival in the positive control (VEGF-A or VEGF-D only) wells. Cell survival is normalized such that survival in negative controls is set to 0 (typically no viable cells were seen in negative controls), while survival in positive controls is set to 100% (typically 300-400 cells/well).

Typically, data is analyzed by one way analysis of variance (ANOVA), with a Bonferroni multiple comparisons test carried out post-hoc to test for differences between individual cultures of chimeric polypeptide alone (to assay binding and activation of the receptor), or chimeric polypeptide+VEGF-A or VEGF-D (to assay inhibition of receptor activation), with VEGF-A or VEGF-D alone (positive control).

As described in Example 1, repetition of the same assay using cells transfected with different chimeric receptors (e.g., VEGFR-3/EpoR) allows screening for activation of different VEGFRs.

VEGFR-2 (KDR) and VEGFR-3 (Flt4) Autophosphorylation Assays.

As an alternative indicator of activity, the ability of a chimeric polypeptide of the invention to stimulate autophosphorylation of a particular VEGF receptor can also be examined. A candidate chimeric polypeptide is added to cells expressing a particular VEGF receptor. The cells are then lysed and immunoprecipitated with anti VEGF receptor antiserum and analyzed by Western blotting using anti phosphotyrosine antibodies to determine chimeric polypeptide induced phosphorylation of the VEGF receptor.

An expression vector comprising a polynucleotide encoding a chimeric VEGF polypeptide of the invention is transfected into an appropriate host cell (e.g., 293 EBNA cells using a calcium phosphate transfection method. About 48 hours after transfection, the growth medium of the transfected cells is changed (e.g., to DMEM medium lacking fetal calf serum) and the cells are incubated (e.g., for 36 more hours) to provide a conditioned medium. Heparin may be included in the culture medium to improve release of recombinant chimeric polypeptides into the medium as described in Example 1. The conditioned medium is collected and centrifuged at 5000×g for 20 minutes, and the supernatant is concentrated.

The concentrated conditioned media is used to stimulate cells expressing a VEGF receptor. For example, PAE-KDR cells (Pajusola et al., Oncogene, 9:3545 55 (1994); Waltenberger et al., J. Biol. Chem., 269: 26988 26995 (1994)) are grown in Ham's F12 medium-10% fetal calf serum (FCS), or confluent NIH 3T3 cells expressing VEGFR-3 are grown in DMEM medium. The cells are starved overnight in DMEM medium or Ham's F12 supplemented with 0.2% bovine serum albumin (BSA), and then incubated for 5 minutes with the unconcentrated, 2 fold, 5 fold, and/or 10 fold concentrated conditioned media. Recombinant human VEGF-A or VEGF-C and conditioned media from mock transfected cells are exemplary controls. In addition to conditional media, purified chimeric polypeptide can be employed in this or other assays described herein.

After stimulation with conditioned media, the cells are washed twice with ice cold Tris-Buffered Saline (TBS) containing 100 mM sodium orthovanadate and lysed in RIPA buffer containing 1 mM phenylmethylsulfonyl fluoride (PMSF), 0.1 U/ml aprotinin and 1 mM sodium orthovanadate. The lysates are sonicated, clarified by centrifugation at 16,000×g for 20 minutes and incubated for 3-6 hours on ice with 3-5 μl of antisera specific for VEGFR-3 or VEGFR-2. Immunoprecipitates are bound to protein A-Sepharose, washed three times with RIPA buffer containing 1 mM PMSF, 1 mM sodium orthovanadate, washed twice with 10 mM Tris-HCl (pH 7.4), and subjected to SDS-PAGE using a 7% gel. Polypeptides are transferred to nitrocellulose by Western blotting and analyzed using PY20 phosphotyrosine-specific monoclonal antibodies (Transduction Laboratories) or receptor-specific antiserum and the ECL detection method (Amersham Corp.).

The ability of a chimeric polypeptide to stimulate autophosphorylation (detected using the anti phosphotyrosine antibodies) is scored as stimulating the receptor. The level of stimulation observed for various concentrations of chimeric polypeptide, relative to known concentrations of VEGF-A, VEGF-D or VEGF-C, provide an indication of the potency of receptor stimulation. Polypeptides that have been shown to bind the receptor, but are incapable of stimulating receptor phosphorylation, are scored as inhibitors. Inhibitory activity can be further assayed by mixing a known receptor agonist such as recombinant VEGF-A or VEGF-C with either media alone or with concentrated conditioned media, to determine if the concentrated conditioned media inhibits VEGF-A mediated or VEGF-C-mediated receptor phosphorylation.

Analysis of Receptor Binding Affinities of Chimeric Polypeptides

The chimeric polypeptides of the present invention may bind more than one VEGFR. Assays may be performed to determine that receptor binding activity of these chimeric polypeptides. For such experiments, the chimeric polypeptide may be expressed in an insect cell system, e.g., SF9 cells, to eliminate contamination with endogenous VEGF-A found in mammalian cells. To measure the relative binding affinities of selected chimeric polypeptide, an ELISA-type approach is used. For example, to examine binding affinity for VEGFR-3, serial dilutions of competing VEGFR-3-IgG fusion proteins and a subsaturating concentration of the candidate chimeric polypeptide tagged with the myc epitope is added to microtitre plates coated with VEGFR-3, and incubated until equilibrium is established. The plates are then washed to remove unbound proteins. Chimeric polypeptide molecules that remain bound to the VEGFR-3 coated plates are detected using an anti-myc antibody conjugated to a readily detectable label e.g., horseradish peroxidase. Binding affinities (EC₅₀) can be calculated as the concentration of competing VEGFR-IgG fusion protein that results in half-maximal binding. These values can be compared with those obtained from analysis of VEGF-A or VEGF-C to determine changes in binding affinity of one or more of the VEGFRs. Similarly, binding to VEGFR-2 is accomplished by using a VEGFR-2-IgG fusion protein, and binding to VEGFR-1 is determined using a VEGFR-1-IgG fusion protein.

Assays for Neuropilin Binding.

Recent results indicate that NRP-1 is a co-receptor for VEGF₁₆₅ binding, forming a complex with VEGFR-2, which results in enhanced VEGF₁₆₅ signaling through VEGFR-2, over VEGF₁₆₅ binding to VEGFR-2 alone, thereby enhancing the biological responses to this ligand (Soker et al., Cell 92: 735-45. 1998). A similar phenomenon may apply to VEGF-C signaling via possible VEGFR-3/NRP-2 receptor complexes. The compositions of the present invention are tested using neuropilin binding assays. Exemplary such assays are described in detail in e.g., U.S. patent application Ser. No. 10/669,176, filed Sep. 23, 2003, U.S. Pat. Nos. 6,428,965 and 6,515,105.

Such assays may employ cells transformed with expression constructs that encode neuropilins. Antibodies and reagents that can be used in neuropilin binding assays are well known to those of skill in the art. See for example, Sema3A-AP which recognizes neuropilin. Competitive binding assays using Sema3 AP and the compositions of the invention will reveal whether the compositions described herein possess neuropilin binding activity. The assays may also use a cell-free complex to determine the binding of the chimerical molecules of the invention competing with binding of VEGF-B₁₆₇, VEGF-C, VEGF-D or processed VEGF-B186 to a receptor. Such a cell-free complex would comprise at least one neuropilin receptor molecule, for example soluble NP-1 (sNP-1) and the composition to be tested. The sNP-1 is defined as a non-membrane bound protein as well as a portion of the receptor, such as the extracellular domain or the ligand-binding fragment of NP-1.

Assays for Endothelial Cell Migration in Collagen Gel.

Both VEGF-A and VEGF-C stimulate endothelial cell migration in collagen gel. The chimeric polypeptides of the invention are examined to determine if they are also capable of stimulating endothelial cell migration in collagen gel, thus providing another indicia of biological activity. Exemplary experiments of such cell migration assays have been described in International Patent Publication No. WO 98/33917, incorporated herein by reference. Briefly, bovine capillary endothelial cells (BCE) are seeded on top of a collagen layer in tissue culture plates. Conditioned media from cells transfected with an expression vector producing the candidate chimeric polypeptide is placed in wells made in collagen gel approximately 4 mm away from the location of the attached BCE cells. The number of BCE cells that have migrated from the original area of attachment in the collagen gel towards the wells containing the chimeric polypeptide is then counted to assess the ability of the chimeric polypeptide to induce cell migration.

BCE cells (Folkman et al., Proc. Natl. Acad. Sci. (USA), 76:5217-5221 (1979)) are cultured as described in Pertovaara et al., J. Biol. Chem., 269:6271-74 (1994). Collagen gels are prepared by mixing type I collagen stock solution (5 mg/ml in 1 mM HCl) with an equal volume of 2×MEM and 2 volumes of MEM containing 10% newborn calf serum to give a final collagen concentration of 1.25 mg/ml. Tissue culture plates (5 cm diameter) are coated with about 1 mm thick layer of the solution, which is allowed to polymerize at 37° C. BCE cells are seeded atop this layer.

For the migration assays, the cells are allowed to attach inside a plastic ring (1 cm diameter) placed on top of the first collagen layer. After 30 minutes, the ring is removed and unattached cells are rinsed away. A second layer of collagen and a layer of growth medium (5% newborn calf serum (NCS)), solidified by 0.75% low melting point agar (FMC BioProducts, Rockland, Me.), are added. A well (3 mm diameter) is punched through all the layers on both sides of the cell spot at a distance of 4 mm, and media containing a chimeric VEGF polypeptide (or media alone or media containing VEGF-A or VEGF-C to serve as controls) is pipetted daily into the wells. Photomicrographs of the cells migrating out from the spot edge are taken, e.g., after six days, through an Olympus CK 2 inverted microscope equipped with phase-contrast optics. The migrating cells are counted after nuclear staining with the fluorescent dye bisbenzimide (1 mg/ml, Hoechst 33258, Sigma).

The number of cells migrating at different distances from the original area of attachment towards wells containing media conditioned by the non-transfected (control) or transfected (mock; chimeric polypeptide; VEGF-C; or VEGF-A) cells are determined 6 days after addition of the media. The number of cells migrating out from the original ring of attachment are counted in five adjacent 0.5 mm×0.5 mm squares using a microscope ocular lens grid and 1× magnification with a fluorescence microscope. Cells migrating further than 0.5 mm are counted in a similar way by moving the grid in 0.5 mm steps.

The ability of a chimeric polypeptide to induce migration of BCE cells is indicative of receptor agonist activity. The number of migrating cells in the presence of a chimeric polypeptide versus a similar concentration of VEGF-A or VEGF-C provides an indication of the potency of agonist activity. Polypeptides that have been shown to bind the receptors expressed on BCE cells, but are incapable of stimulating migration, are scored as potential inhibitors. Inhibitory activity can be further assayed by mixing a known receptor agonist such as recombinant VEGF-A or VEGF-C with either media alone or with concentrated conditioned media, to determine if the concentrated conditioned media inhibits VEGF-A-mediated or VEGF-C-mediated BCE migration.

Assay for Induction of Vascular Permeability

Both VEGF-A and VEGF-C are capable of increasing the permeability of blood vessels. The chimeric polypeptides of the invention are assayed to determine which of these proteins possess this biological activity and which inhibit it. For example, vascular permeability assays according to Miles and Miles, J. Physiol 118:228-257 (1952), incorporated herein in its entirety, are used to analyze the chimeric polypeptide . Briefly, following intravenous injection of a vital dye, such as pontamine sky blue, animals such as guinea pigs are injected intradermally with a composition containing the candidate chimeric polypeptide being examined. For controls, media alone or media containing VEGF-A or VEGF-C is injected in the same manner. After a period of time, the accumulation of dye at the injection site on the skin is measured. Those chimeric polypeptides that increase permeability will result in greater accumulation of dye at the injection site as compared to those chimeric polypeptides that fail to induce vascular permeability.

In a variation of this assay, chimeric polypeptides that are suspected of being inhibitors of VEGF-A or VEGF-C are first mixed with VEGF-A or with VEGF-C at varying ratios (e.g., 50:1, 10:1, 5:1, 2:1, 1:1, 1:2, 1:5, 1:10) and the mixtures are injected intradermally into the animals. In this manner, the ability of the chimeric polypeptide to inhibit VEGF-A-mediated or VEGF-C-mediated vascular permeability is assayed.

Endothelial Cell Proliferation Assays

The mitogenic activity of the chimeric polypeptides can be examined using endothelial cell proliferation assays such as that described in Breier et al., Dev 114:521-532 (1992), incorporated herein in its entirety. The chimeric polypeptides are expressed in a mammalian cell line e.g., COS cells. Culture supernatants are then collected and assayed for mitogenic activity on bovine aortic endothelial (BAE) cells by adding the supernatants to the BAE cells. After three days, the cells are dissociated with trypsin and counted using a cytometer to determine any effects of the chimeric polypeptide on the proliferative activity of the BAE cells. As negative controls, DMEM supplemented with 10% FCS and the conditioned media from untransfected COS cells or from COS cells transfected with vector alone can be used. Supernatants from cells transfected with constructs expressing proteins that have been shown to induce proliferation of BAE cells (e.g., VEGF-A) can be used as a positive control.

Induction of In Vivo growth of Lymphatic and/or Blood Vessels in Skin of Transgenic Mice

Experiments may be conducted in transgenic mice to analyze the specific effects of overexpression of chimeric polypeptides in tissues. The physiological effects in vivo provide an indication of receptor activation/inhibition profile and an indication of the potential therapeutic action of a chimeric polypeptide. In one variation, the human K14 keratin promoter which is active in the basal cells of stratified squamous epithelia [Vassar et al., Proc. Natl. Acad. Sci. (USA), 86:1563-1567 (1989)], is used as the expression control element in the recombinant chimeric polypeptide transgene. The vector containing the K14 keratin promoter is described in Vassar et al., Genes Dev., 5:714-727 (1991) and Nelson et al., J. Cell Biol. 97:244-251 (1983).

A DNA fragment containing the K14 promoter, chimeric polypeptide encoding cDNA, and K14 polyadenylation signal is isolated, and injected into fertilized oocytes of the FVB-NIH mouse strain. The injected zygotes are transplanted to oviducts of pseudopregnant C57BL/6×DBA/2J hybrid mice. The resulting founder mice are then analyzed for the presence of the transgene by polymerase chain reaction of tail DNA using appropriate primers or by Southern analysis.

These transgenic mice are then examined for evidence of angiogenesis or lymphangiogenesis in the skin, such as the lymphangiogenesis seen in transgenic mice that overexpress VEGF-C [see International Publication WO98/33917]. Histological examination of K14-VEGF-C transgenic mice showed that in comparison to the skin of wildtype littermates, the dorsal dermis was atrophic and connective tissue was replaced by large lacunae devoid of red cells, but lined with a thin endothelial layer. These distended vessel-like structures resembled those seen in human lymphangiomas. The number of skin adnexal organs and hair follicles were reduced. In the snout region, an increased number of vessels was also seen.

Examination of the vessels in the skin of the transgenic mice using antibodies that recognize proteins specific for either blood or lymphatic vessels can further verify the identity of these vessels. Collagen types IV, XVIII [Muragaki et al., Proc. Natl. Acad. Sci. USA, 92: 8763-8776 (1995)] and laminin are expressed in vascular endothelial cells while desmoplakins I and II (Progen) are expressed in lymphatic endothelial cells. See Schmelz et al., Differentiation, 57: 97-117 (1994).

In addition, the chimeric molecules can be co-expressed with Ang-1 or PDGF-B in transgenic mice, to determine whether abnormalities of lymphatic vesels observed in K14-VEGF-C mice can be corrected.

Assays for Determining Modulation of Myelopoiesis

Overexpression of VEGF-C in the skin of K14-VEGF-C transgenic mice correlates with a distinct alteration in leukocyte populations [see International Publication W098/33917]. Notably, the measured populations of neutrophils were markedly increased in the transgenic mice. The effects of the chimeric polypeptides on hematopoiesis can be analyzed using fluorescence-activated cell sorting analysis using antibodies that recognize proteins expressed on specific leukocyte cell populations. Leukocyte populations are analyzed in blood samples taken from the F1 transgenic mice described above, and from their non-transgenic littermates. Alterations in leukocyte populations has numerous therapeutic indications, such as stimulating an immune response to pathogens, recovery of the immune system following chemotherapy or other suppressive therapy, or in the case of inhibitors, beneficial immunosuppression (e.g., to prevent graft-versus-host-disease or autoimmune disorders.) Use of molecules of the invention for these therapeutic indications is specifically contemplated.

Assays to Determine Effect on Growth and Differentiation of Human CD34+ Progenitor Cells In Vitro

Addition of VEGF-C to cultures of cord blood CD34+ cells induces cell proliferation. Co-culture of GM-CSF, IL-3, GM-CSF+IL-3, or GM-CSF+SCF with VEGF-C leads to an enhancement of proportions of myeloid cells [see International Publication WO98/33917]. Chimeric polypeptides of the invention can also be examined for their ability to induce growth of CD34+ progenitor cells in vitro. Human CD34+ progenitor cells (HPC, 10×103) are isolated from bone marrow or cord blood mononuclear cells using the MACS CD34 Progenitor cell Isolation Kit (Miltenyi Biotec, Bergish Gladbach, Germany), according to the instructions of the manufacturer and cultured in RPMI 1640 medium supplemented with L-glutamine (2.5 mM), penicillin (125 IE/ml), streptomycin (125 μg/ml) and pooled 10% umbilical cord blood (CB) plasma at 37° C. in a humidified atmosphere in the presence of 5% CO2 for seven days, with or without chimeric polypeptide at concentrations ranging from 10 ng/ml to 1 μg/ml. After seven days, total cell number is evaluated in each culture.

The co-stimulatory effect of chimeric polypeptides in cultures either supplemented with recombinant human stem cell factor (rhSCF, 20 ng/ml PreproTech, Rocky Hill, N.Y.) alone or a combination of granulocyte macrophage colony stimulating factor (rhGM-CSF, 100 ng/ml, Sandoz, Basel, Switzerland) plus SCF can also be examined. Experiments can also be conducted to analyze the co-stimulatory effects of chimeric polypeptide on total cell yields of serum-free cultures of CB CD34+ HPC cells supplemented with either GM-CSF alone, IL-3 (rhIL-3, 100 U/ml, Bearing BAG, Mar burg, Germany) alone; or a combination of GM-CSF plus IL-3.

Cells from the (7 day) plasma-supplemented cultures described above are also analyzed for the expression of the early granulomonocytic marker molecules lysozyme (LZ) and myeloperoxidase (MPO) as well as the lipopolysaccharide (LPS) receptor CD 14 using immunofluorescence.

In another series of experiments, CD34+ cells are cultured in medium supplemented with 50 ng/ml M-CSF, with or without 100 ng/ml chimeric polypeptide, for seven days. After seven days, the cultures were analyzed to determine the percentages of CD34+ cells and mean fluorescence intensity.

Analysis of Chimeric Polypeptides Using CAM Assays

The choroallantoic membrane (CAM) assay described in e.g., Oh et al., Dev Biol 188 :96-109 (1997), incorporated herein in its entirety, is a commonly used method to examine the in vivo effects of angiogenic factors. Using this assay, VEGF growth factors including both VEGF-A and VEGF-C have been shown to induce the development of blood vessels [Oh et al., Dev Biol 188:96-109 (1997)]. Thus, this method can be used to study the angiogenic properties of the chimeric polypeptide.

Briefly, on day 4 of development, a window is cut out into the eggshell of chick or quail eggs. The embryos are checked for normal development, the window in the eggshell is sealed with cellotape, and the eggs are incubated until day 13 of development. Approximately 3.3 μg of chimeric polypeptide dissolved in 5 μl of distilled water is added to Thermanox coverslips (Nunc, Naperville, Ill.), which have been cut into disks with diameters of approximately 5 mm, and air dried. Disks without added protein are used as controls. The dried disks are then applied on the chorioallantoic membrane (CAM) of the eggs. After 3 days, the disks are removed and fixed in 3% glutaraldehyde and 2% formaldehyde and rinsed in 0.12 M sodium cacodylate buffer. The fixed specimens are photographed and embedded in Epon resin (Serva, Germany) for semi-(0.75 μm) and ultrathin (70 nm) sectioning. Both semi- and ultrathin sections are cut using an Ultracut S (Leika, Germany). Ultrathins sections are analyzed by an EM 10 (Zeiss, Germany). Specimens are then analyzed for evidence of growth of new capillaries, which would indicate that the chimeric polypeptide being examined is capable of stimulating angiogenesis.

Angiogenesis in Tissue Ischemia

Utility of chimeric polypeptides of the invention in treating ischemic tissue, such as limb ischemia due to insufficient circulation, is analyzed using recognized assays. The efficacy of the chimeric polypeptides in such indications may be determined using model for such ischemia, such as for example a rabbit model for ischemia has previously been described (Bauters et al., Am J. Physiol. 267:H1263-1271, 1996; Pu et al., J. Invest. Surgery, 7:49-60, 1994). These animals are anesthetized and the femoral artery of on hindlimb is excised from its proximal origin as a branch of the external iliac artery to the point where it bifurcates into the saphenous and popliteal arteries. As a result of this procedure, the blood flow to the ischemic limb is dependent on collateral vessels originating from the internal iliac artery Takeshita et al., Circulation, 90:II-228-II-234, 1994). The animal is allowed a 10-day post-operative recovery period. During this period, endogenous collateral vessels develop. After the recovery period, the baseline physiological parameters, such as blood pressure, intravascular blood flow, iliac angiography and capillary vessel density is determined. Methods for determining these baseline physiological characteristics are detailed in Witzenbichler et al., (Am. J. Path. 153:381-394, 1998). Lymphatic vessels also are analyzed as described in Example 2.

After obtaining the baseline physiological characteristics of the animal, the model animal is treated with an intra-arterial bolus of a chimeric polypeptide of the present invention. Preferably, the bolus comprises the equivalent of 500 μg of VEGF-C in an appropriate volume, e.g., 3 ml, of phosphate buffered saline (PBS) containing 0.1% rabbit serum albumin (RSA). The chimeric protein is administered over a period of 1 to 5 minutes through a catheter positioned in the internal iliac artery of the ischemic limb. The catheter is then washed with an equal volume of PBS containing RSA. The physiological parameters discussed above are then monitored at suitable intervals after administration of the chimeric polypeptides.

In an alternative embodiment, the ischemic model is treated using gene therapy with either naked DNA comprising polynucleotides that encode the chimeric polypeptides of the present invention or, preferably, gene therapy vectors described herein that encode a chimeric polypeptide of the present invention. Adenoviral gene therapy vectors are particularly preferred. In such gene therapy embodiments, the internal iliac artery of the ischemic limb of the animal is transfected with the naked DNA or the adenoviral or other gene therapy vector using e.g., a 2.0 mm balloon catheter (Slider with Hydroplus, Boston Scientific, Mass.). The angioplasty balloon is preferably prepared ex vivo by first advancing the deflated balloon througha Teflon sheath (Boston Scientific) and applying the gene therapy composition to the layer of hydrogel coating the external surface of the inflated balloon. The balloon is then retracted back into its protective sheath. The sheath and the angioplasty catheter are introduced via the right carotid artery and advanced to the lower abdominal aorta using an appropriate guide-wire. The balloon catheter is advanced to the internal iliac artery of the ischemic limb and inflated to administer the gene therapy composition locally at the ischemic limb. The balloon catheter is then deflated and withdrawn.

The above methods may be performed with controls that comprise no VEGF-related composition and other controls which comprise VEGF-C, VEGF-D or even VEGF-A.

The above studies are described with respect to a rabbit model for ischemia. Similar studies may be conducted in models of ischemic heart disease, such as those described by Kastrup et al., (Curr. Gene Ther., 3(3):197-206, 2003), and Khan et al., (Gene Ther. 10(4):258-91, 2003).

A further indication for the compositions of the present invention may be demonstrated in vivo in rabbit restenosis models, to demonstrate the efficacy of the compositions for the prevention of post-angioplasty restenosis. The animal models typically are rabbits, but other mammals may be tested. A first group of rabbits is fed a 0.25% cholesterol diet for two weeks, then subjected to balloon denudation of the aorta, then subjected three days later to the therapeutic compositions to be tested. Animals are sacrificed 2 or 4 weeks after the initiation of therapy. The compositions to be tested include VEGF-C, or VEGF-D or chimeric compositions of the invention that comprise VEGF-C or VEGF-D, either alone or in combination with a PDGF inhibitor (for example an α-PDGF-A antibody; α-PDGF-B antibody, α-PDGF-C antibody, α-PDGF-D antibody, a α-PDGFR-alpha antibody or a α-PDGFR-beta antibody or a short interfering RNA molecule directed to one or more of these targets) or with one or more other smooth muscle cell growth inhibitors. Polypeptide therapy or gene therapy is contemplated. As a gene therapy control, the vector of choice carries the LacZ gene.

In the first group of rabbits, the whole aorta, beginning from the tip of the arch, is denuded using a 4.0 F arterial embolectomy catheter (Sorin Biomedical, Irvine, Calif.). The catheter is introduced via the right iliac artery up to the aortic arch and inflated, and the aorta is denuded twice.

Three hours before sacrifice, the animals are injected intravenously with 50 mg of BrdU dissolved in 40% ethanol. After the sacrifice, the aortic segment where the gene transfer had been performed is removed, flushed gently with saline, and divided into five equal segments. The proximal segment is snap frozen in liquid nitrogen and stored at −70° C. The next segment is immersion-fixed in 4% paraformaldehyde/15% sucrose (pH 7.4) for 4 hours, rinsed in 15% sucrose (pH 7.4) overnight, and embedded in paraffin. The medial segment is immersion-fixed in 4% paraformaldehyde/phosphate buffered saline (PBS) (pH 7.4) for 10 minutes, rinsed 2 hours in PBS, embedded in OCT compound (Miles), and stored at −70° C. The fourth segment is immersion-fixed in 70% ethanol overnight and embedded in paraffin. The distal segment is directly stained for β-galactosidase activity in X-GAL staining solution at +37° C. for 16 hours, immersion-fixed in 4% paraformaldehyde/15% sucrose (pH 7.4) for 4 hours, rinsed in 15% sucrose overnight, and embedded in paraffin. Paraffin sections are used for immunocytochemical detection of smooth muscle cells (SMC), macrophages, and endothelium. BrdU-positive cells are detected according to manufacturer's instructions. Morphometric analysis performed using haematoxylin-eosin stained paraffin sections using image analysis software. Intima/media (I/M) ratio is used as a parameter for intimal thickening.

Histological analysis of the balloon-denuded mice is taken. Compositions that are effective at inhibiting restenosis will reveal that control groups (i.e., those groups without the compositions that comprise the VEGF-C related compositions) have an I/M ratio of that is higher than the ratio from those animals treated with the VEGF-C-based therapeutic compositions.

The BrdU labeling will permit analysis of smooth muscle cell proliferation in treated versus control animals. SMC proliferation is expected to be reduced in the treated population. A more detailed description of assays and compositions for treating restenosisis contained in international application no. PCT/US99/24054, published as WO 00/24412 incorporated herein by reference in its entirety.

It should be understood that the foregoing description relates to preferred embodiments of the invention and equivalents and variations that will be apparent to the reader are also intended as aspects of the invention. The references cited herein throughout, to the extent that they provide exemplary procedural or other details supplementary to those set forth herein, are all specifically incorporated herein by reference.

1. A compound comprising the formula X-B-Z or Z-B-X,
wherein X binds Vascular Endothelial Growth Factor Receptor 3 (VEGFR-3) and comprises an amino acid sequence at least 90% identical to a VEGFR-3 ligand selected from the group consisting of:
(a) the prepro-VEGF-C amino acid sequence set forth in SEQ ID NO: 2;
(b) fragments of (a) that bind VEGFR-3;
(c) the prepro-VEGF-D amino acid sequence set forth in SEQ ID NO: 4; and
(d) fragments of (c) that bind VEGFR-3;
wherein Z comprises a heparin-binding amino acid sequence; and
wherein B comprises a covalent attachment linking X to Z.

2. The compound of claim 1, wherein X comprises an amino acid sequence at least 95% identical to a VEGFR-3 ligand selected from the group consisting of:
(a) the prepro-VEGF-C amino acid sequence set forth in SEQ ID NO: 2; and
(b) fragments of (a) that bind VEGFR-3.

3. The compound of claim 1, wherein when X comprises an amino acid sequence at least 95% identical to a VEGFR-3 ligand selected from the group consisting of:
(a) the prepro-VEGF-D amino acid sequence set forth in SEQ ID NO: 4; and
(b) fragments of (a) that bind VEGFR-3.

4. The compound of claim 1, wherein the compound binds Vascular Endothelial Growth Factor Receptor 2 (VEGFR-2).

5. The compound of claim 1, wherein the heparin binding amino acid sequence comprises an amino acid sequence at least 90% identical to a sequence selected from the group consisting of:
(a) amino acids 142-165 of the VEGF₂₀₆ (SEQ ID NO: 5);
(b) amino acids 183 to 226 of the VEGF₂₀₆ (SEQ ID NO: 5);
(c) amino acids 142-165 (SEQ ID NO: 5) joined directly to amino acids 183-226 (SEQ ID NO: 5) of the VEGF₂₀₆;
(d) amino acids 142 to 226 of the VEGF₂₀₆ (SEQ ID NO: 5);
(e) amino acids 138 to 182 of the VEGF-B₁₆₇ sequence set forth in SEQ ID NO: 8;
(f) amino acids 193 to 213 of the PlGF-3 sequence set forth in SEQ ID NO: 15;
(g) amino acids of 142 to 162 of the PIGF-2 sequence set forth in SEQ ID NO:69;
(h) fragments of (a)-(g) that bind heparin.

6. The compound of claim 1, wherein X-B-Z or Z-B-X, is a polypeptide.

7. The compound of claim 6, further comprising a signal peptide at the amino terminus of the polypeptide, wherein the signal peptide directs secretion of a polypeptide comprising X-B-Z or Z-B-X from a cell that expresses the polypeptide.

8. The compound of claim 1, wherein B is selected from the group consisting of:
(a) a peptide bond; and
(b) a peptide linker up to 500 amino acids.

9. The compound of claim 1, wherein B comprises a peptide bond that is cleavable by an agent that fails to cleave the amino acid sequence X that binds VEGFR-3.

10. The compound of claim 9, wherein peptide bond is cleaved by a protease.

11. The compound of claim 9, wherein B comprises an amino acid sequence that contains a protease cleavage site selected from the group consisting of a Factor Xa cleavage site, an enterokinase cleavage site, a thrombin cleavage site, a TEV cleavage site, and a PreScission cleavage site.

12. The compound of claim 1, wherein B comprises an amino acid sequence of at least four amino acids from a VEGF-C or VEGF-D amino acid sequence, wherein the at least four amino acids are cleaved in vivo to separate an amino-terminal propeptide that includes the heparin binding amino acid sequence from a mature VEGF-C or VEGF-D protein.

13. The compound of claim 1 wherein B is selected from the group consisting of a peptide bond and a peptide linker up to 50 amino cells in length.

14. The compound of claim 1, wherein X comprises an amino acid sequence at least 95% identical to the prepro-VEGF-C amino acid sequence set forth in SEQ ID NO: 2 or to a fragment thereof that binds VEGFR-3,
with the proviso that the cysteine corresponding to amino acid position 156 of SEQ ID NO: 2 has been deleted or replaced with an amino acid other than cysteine, and the resultant amino acid sequence binds VEGFR-3 but has reduced VEGFR-2 binding.

15. The compound of claim 1, wherein X comprises an amino acid sequence identical to the prepro-VEGF-C amino acid sequence set forth in SEQ ID NO: 2 or to a fragment thereof that binds VEGFR-3.

16. The compound of claim 1, wherein X comprises an amino acid sequence identical to the prepro-VEGF-C amino acid sequence set forth in SEQ ID NO: 2 or to a fragment thereof that binds VEGFR-3,
with the proviso that the cysteine corresponding to amino acid position 156 of SEQ ID NO: 2 has been deleted or replaced with an amino acid other than cysteine, and the resultant amino acid sequence binds VEGFR-3 but has reduced VEGFR-2 binding.

17. The compound of claim 1, wherein X comprises an amino acid sequence identical to the prepro-VEGF-D amino acid sequence set forth in SEQ ID NO: 4 or to a fragment thereof that binds VEGFR-3.

18. The compound of claim 1, wherein the compound further includes a peptide tag (e.g., a polyhistidine tag) to facilitate purification.

19. A composition comprising a compound of claim 1 in a pharmaceutically acceptable carrier.

20. A polynucleotide comprising a nucleotide sequence that encodes a compound of claim 6.

21. A polynucleotide of claim 20, wherein the polynucleotide further comprises a nucleotide sequence that encodes a signal peptide fused in-frame with the polypeptide.

22. A vector comprising a polynucleotide of claim 20.

23. An expression vector comprising a polynucleotide of claim 20 operably linked to an expression control sequence.

24. An expression vector of claim 23, wherein the expression control sequence is an endothelial cell specific promoter.

25. A vector of claim 24, selected from the group consisting of replication deficient adenoviral vectors, adeno-associated viral vectors, and lentivirus vectors.

26. A composition comprising a polynucleotide of claim 20 and a pharmaceutically acceptable carrier, diluent or excipient.

27. A composition comprising a vector of claim 23 and a pharmaceutically acceptable carrier, diluent or excipient.

28. A host cell transformed or transfected with a polynucleotide of claim 20.

29. A host cell transformed or transfected with a vector of claim 23.

30. A host cell that expresses a compound of claim 6.

31. A method of modulating the growth of mammalian endothelial cells or mammalian endothelial precursor cells, comprising contacting the cells with a composition comprising a member selected from the group consisting of:
(a) a polypeptide compound of claim 6;
(b) a polynucleotide that encodes (a);
(c) an expression vector containing (b) operatively linked to an expression control sequence; and
(d) a cell transformed or transfected with (b) or (c) that expresses the polypeptide of (a).

32. A method of claim 31, wherein the contacting comprises administering the composition to a mammalian subject in an amount effective to modulate endothelial cell growth in vivo.

33. A method of claim 32, wherein the mammalian subject is a human.

34. A method according to claim 33, wherein the subject has lymphedema.

35. A method of modulating the growth of mammalian hematopoietic progenitor cells, comprising contacting the cells with a composition comprising a member selected from the group consisting of:
(a) a polypeptide compound of claim 6;
(b) a polynucleotide that encodes (a);
(c) an expression vector containing (b) operatively linked to an expression control sequence; and
(d) a cell transformed or transfected with (b) or (c) that expresses the polypeptide compound of (a).

36. A method for activation of VEGFR-3? comprising contacting cells that express VEGFR-3 with a composition comprising a compound of claim 1.

37. A method of stimulating lymphangiogenesis in a mammal comprising contacting said mammal with, and/or administering to said mammal, a composition comprising a member selected from the group consisting of:
(a) a polypeptide compound of claim 6;
(b) a polynucleotide that encodes (a);
(c) an expression vector containing (b) operatively linked to an expression control sequence; and
(d) a cell transformed or transfected with (b) or (c) that expresses the polypeptide compound of (a).

38. A method of stimulating angiogenesis in a mammal comprising contacting said mammal with a composition comprising a member selected from the group consisting of:
(a) a polypeptide compound of claim 4;
(b) a polynucleotide that encodes (a);
(c) an expression vector containing (b) operatively linked to an expression control sequence; and
(d) a cell transformed or transfected with (b) or (c) that expresses the polypeptide (a).
